SEQUENCE LISTING (139 sequences)

SEQ ID NO: 1 - protein, 11 aa, Artificial Sequence
  Synthetic Construct
  VARIANT 1: Xaa = Phe or Tyr
  VARIANT 5: Xaa = Asp or Gly
  VARIANT 7: Xaa = Ala, Gly or Trp
XTFSXYXIHWV

SEQ ID NO: 2 - protein, 12 aa, Artificial Sequence
  Synthetic Construct
  VARIANT 4: Xaa = Ser or Thr
  VARIANT 7, 8: Xaa = His or Tyr
  VARIANT 10: Xaa = Ala, Asp, or Ser
YSIXSGXXWXWI

SEQ ID NO: 3 - protein, 13 aa, Artificial Sequence
  Synthetic Construct
  VARIANT 11: Xaa = Gly or Ser
FSLSTGGVAVXWI

SEQ ID NO: 4 - protein, 20 aa, Artificial Sequence
  Synthetic Construct
  VARIANT 3: Xaa = Asp or Glu
  VARIANT 5: Xaa = Ser or Tyr
  VARIANT 14: Xaa = Pro or Gln
IGXIXHSGSTYYSXSLKSRV

SEQ ID NO: 5 - protein, 21 aa, Artificial Sequence
  Synthetic Construct
  VARIANT 3: Xaa = Ile or Trp
  VARIANT 8, 10: Xaa = Gly or Ser
  VARIANT 12: Xaa = Lys or Asn
IGXISPSXGXTXYAQKFQGRV

SEQ ID NO: 6 - protein, 21 aa, Artificial Sequence
  Synthetic Construct
  VARIANT 3: Xaa = Ala, Gly, or Ser
  VARIANT 7: Xaa = Ser or Tyr
  VARIANT 9: Xaa = Gly or Ser
  VARIANT 10: Xaa = Ser or Thr
VSXISGXGXXTYYADSVKGRF

SEQ ID NO: 7 - protein, 9 aa, Artificial Sequence
  Synthetic Construct
  VARIANT 3: Xaa = Gly, Arg, or Ser
  VARIANT 4: Xaa = Ala, Ile, or Tyr
  VARIANT 5: Xaa = Asp, Val, or Tyr
  VARIANT 6: Xaa = Ala, Glu, or Tyr
  VARIANT 9: Xaa = Ile or Tyr
ARXXXXFDX

SEQ ID NO: 8 - protein, 10 aa, Artificial Sequence
  Synthetic Construct
  VARIANT 3: Xaa = Asp or Leu
  VARIANT 5: Xaa = Phe or Tyr
  VARIANT 10: Xaa = Val or Tyr
ARXGXGYFDX

SEQ ID NO: 9 - protein, 12 aa, Artificial Sequence
  Synthetic Construct
  VARIANT 3: Xaa = Leu or Arg
  VARIANT 4: Xaa = Ile or Pro
  VARIANT 5: Xaa = Ala or Tyr
  VARIANT 6: Xaa = Ser or Thr
  VARIANT 8: Xaa = Thr or Tyr
  VARIANT 9: Xaa = Ala or Tyr
ARXXXXAXXFDY

SEQ ID NO: 10 - protein, 17 aa, Artificial Sequence
  Synthetic Construct
  VARIANT 4: Xaa = Ile or Val
  VARIANT 5: Xaa = Ala or His
  VARIANT 6: Xaa = Pro or Ser
  VARIANT 13: Xaa = Asp or Tyr
  VARIANT 17: Xaa = Phe or Val
ARDXXXGSSGYYXGFDX

SEQ ID NO: 11 - protein, 11 aa, Artificial Sequence
  Synthetic Construct
  VARIANT 5, 7: Xaa = Gly or Ser
  VARIANT 6: Xaa = Ile or Val
  VARIANT 9: Xaa = Ser or Tyr
  VARIANT 11: Xaa = Ala or Asn
RASQXXXSXLX

SEQ ID NO: 12 - protein, 12 aa, Artificial Sequence
  Synthetic Construct
  VARIANT 5: Xaa = Ser or Thr
  VARIANT 7: Xaa = Phe, Arg, or Ser
  VARIANT 8: Xaa = Gly or Ser
  VARIANT 10: Xaa = Phe or Tyr
RASQXVXXRXLA

SEQ ID NO: 13 - protein, 15 aa, Artificial Sequence
  Synthetic Construct
  VARIANT 4: Xaa = Glu or Gln
  VARIANT 9: Xaa = Asp, Phe, His, or Tyr
  VARIANT 11: Xaa = Phe, Ile, or Lys
  VARIANT 15: Xaa = Ala, Asp, or His
RASXSVDFXGXSFLX

SEQ ID NO: 14 - protein, 9 aa, Artificial Sequence
  Synthetic Construct
  VARIANT 1: Xaa = Ala or Asp
  VARIANT 4: Xaa = Asn, Ser or Thr
  VARIANT 5: Xaa = Leu or Arg
  VARIANT 6: Xaa = Ala, Glu, or Gln
  VARIANT 7: Xaa = Ser or Thr
  VARIANT 9: Xaa = Ile or Val
XASXXXXGX

SEQ ID NO: 15 - protein, 11 aa, Artificial Sequence
  Synthetic Construct
  VARIANT 3: Xaa = Glu, Gln, or Val
  VARIANT 4: Xaa = His or Gln
  VARIANT 5: Xaa = Ala, Gly, His, Arg, or Ser
  VARIANT 6: Xaa = Asp, Leu, Ser, or Tyr
  VARIANT 7: Xaa = Glu, Gly, Pro, Gln, or Ser
  VARIANT 8: Xaa = Leu, Thr, Val, or Trp
  VARIANT 10: Xaa = Phe, Leu, Pro, Trp, or Tyr
YCXXXXXXPXT

SEQ ID NO: 16 - protein, 12 aa, Artificial Sequence
  Synthetic Construct
  VARIANT 5: Xaa = Ser or Tyr
  VARIANT 6: Xaa = Asp or Tyr
  VARIANT 7: Xaa = Gln or Tyr
YCQQXXXWPPWT

SEQ ID NO: 17 - protein, 13 aa, Artificial Sequence
  Synthetic Construct
  VARIANT 4: Xaa = His or Gln
  VARIANT 6: Xaa = Thr or Val
  VARIANT 11: Xaa = Glu or Val
YCQXYXSSPPXYT

SEQ ID NO: 18 - protein, 11 aa, Artificial Sequence
  Synthetic Construct
FTFSDYAIHWV

SEQ ID NO: 19 - protein, 12 aa, Artificial Sequence
  Synthetic Construct
YSITSGYYWAWI

SEQ ID NO: 20 - protein, 11 aa, Artificial Sequence
  Synthetic Construct
FTFSDYGIHWV

SEQ ID NO: 21 - protein, 12 aa, Artificial Sequence
  Synthetic Construct
YSISSGYHWDWI

SEQ ID NO: 22 - protein, 11 aa, Artificial Sequence
  Synthetic Construct
FTFSDYWIHWV

SEQ ID NO: 23 - protein, 12 aa, Artificial Sequence
  Synthetic Construct
YSISSGYHWSWI

SEQ ID NO: 24 - protein, 13 aa, Artificial Sequence
  Synthetic Construct
FSLSTGGVAVSWI

SEQ ID NO: 25 - protein, 13 aa, Artificial Sequence
  Synthetic Construct
FSLSTGGVAVGWI

SEQ ID NO: 26 - protein, 11 aa, Artificial Sequence
  Synthetic Construct
FTFSGYAIHWV

SEQ ID NO: 27 - protein, 11 aa, Artificial Sequence
  Synthetic Construct
YTFSGYGIHWV

SEQ ID NO: 28 - protein, 11 aa, Artificial Sequence
  Synthetic Construct
YTFSGYAIHWV

SEQ ID NO: 29 - protein, 12 aa, Artificial Sequence
  Synthetic Construct
YSITSGHYWSWI

SEQ ID NO: 30 - protein, 21 aa, Artificial Sequence
  Synthetic Construct
IGIISPSSGSTNYAQKFQGRV

SEQ ID NO: 31 - protein, 21 aa, Artificial Sequence
  Synthetic Construct
VSSISGSGSTTYYADSVKGRF

SEQ ID NO: 32 - protein, 20 aa, Artificial Sequence
  Synthetic Construct
IGEIYHSGSTYYSPSLKSRV

SEQ ID NO: 33 - protein, 21 aa, Artificial Sequence
  Synthetic Construct
VSGISGYGGSTYYADSVKGRF

SEQ ID NO: 34 - protein, 21 aa, Artificial Sequence
  Synthetic Construct
IGWISPSGGGTKYAQKFQGRV

SEQ ID NO: 35 - protein, 20 aa, Artificial Sequence
  Synthetic Construct
LARIDWDDDKYYSTSLKSRL

SEQ ID NO: 36 - protein, 21 aa, Artificial Sequence
  Synthetic Construct
VSAISGYGSTTYYADSVKGRF

SEQ ID NO: 37 - protein, 21 aa, Artificial Sequence
  Synthetic Construct
IGIISPSGGGTKYAQKFQGRV

SEQ ID NO: 38 - protein, 21 aa, Artificial Sequence
  Synthetic Construct
IGIISPSGGSTKYAQKFQGRV

SEQ ID NO: 39 - protein, 20 aa, Artificial Sequence
  Synthetic Construct
IGDISHSGSTYYSQSLKSRV

SEQ ID NO: 40 - protein, 17 aa, Artificial Sequence
  Synthetic Construct
ARDIHSGSSGYYYGFDV

SEQ ID NO: 41 - protein, 10 aa, Artificial Sequence
  Synthetic Construct
ARDGFGYFDY

SEQ ID NO: 42 - protein, 17 aa, Artificial Sequence
  Synthetic Construct
ARDVAPGSSGYYDGFDF

SEQ ID NO: 43 - protein, 13 aa, Artificial Sequence
  Synthetic Construct
ARHSYYGSGNFDY

SEQ ID NO: 44 - protein, 9 aa, Artificial Sequence
  Synthetic Construct
ARGAYEFDY

SEQ ID NO: 45 - protein, 9 aa, Artificial Sequence
  Synthetic Construct
ARSYVYFDY

SEQ ID NO: 46 - protein, 12 aa, Artificial Sequence
  Synthetic Construct
ARRIATATYFDY

SEQ ID NO: 47 - protein, 12 aa, Artificial Sequence
  Synthetic Construct
ARLPYSAYAFDY

SEQ ID NO: 48 - protein, 7 aa, Artificial Sequence
  Synthetic Construct
ARHPFAY

SEQ ID NO: 49 - protein, 9 aa, Artificial Sequence
  Synthetic Construct
ARRIDAFDI

SEQ ID NO: 50 - protein, 8 aa, Artificial Sequence
  Synthetic Construct
ARLYDVAY

SEQ ID NO: 51 - protein, 10 aa, Artificial Sequence
  Synthetic Construct
ARLGYGYFDV

SEQ ID NO: 52 - protein, 11 aa, Artificial Sequence
  Synthetic Construct
ARGSRTGYFDY

SEQ ID NO: 53 - protein, 15 aa, Artificial Sequence
  Synthetic Construct
RASESVDFFGISFLA

SEQ ID NO: 54 - protein, 10 aa, Artificial Sequence
  Synthetic Construct
SASSSVSYVY

SEQ ID NO: 55 - protein, 11 aa, Artificial Sequence
  Synthetic Construct
RASQGIGSSLA

SEQ ID NO: 56 - protein, 15 aa, Artificial Sequence
  Synthetic Construct
RASESVDFFGKSFLH

SEQ ID NO: 57 - protein, 12 aa, Artificial Sequence
  Synthetic Construct
RASQSVSSRFLA

SEQ ID NO: 58 - protein, 12 aa, Artificial Sequence
  Synthetic Construct
RASQSVRGRFLA

SEQ ID NO: 59 - protein, 12 aa, Artificial Sequence
  Synthetic Construct
RASQTVFSRYLA

SEQ ID NO: 60 - protein, 11 aa, Artificial Sequence
  Synthetic Construct
RASQGVSSYLA

SEQ ID NO: 61 - protein, 15 aa, Artificial Sequence
  Synthetic Construct
RASQSVDFYGISFLD

SEQ ID NO: 62 - protein, 15 aa, Artificial Sequence
  Synthetic Construct
RASQSVDFDGFSFLH

SEQ ID NO: 63 - protein, 15 aa, Artificial Sequence
  Synthetic Construct
RASQSVDFHGKSFLH

SEQ ID NO: 64 - protein, 15 aa, Artificial Sequence
  Synthetic Construct
RASQSVDFYGISFLH

SEQ ID NO: 65 - protein, 11 aa, Artificial Sequence
  Synthetic Construct
RASQSISSYLN

SEQ ID NO: 66 - protein, 9 aa, Artificial Sequence
  Synthetic Construct
DASNRATGI

SEQ ID NO: 67 - protein, 9 aa, Artificial Sequence
  Synthetic Construct
DASSLESGV

SEQ ID NO: 68 - protein, 9 aa, Artificial Sequence
  Synthetic Construct
DASNLETGV

SEQ ID NO: 69 - protein, 9 aa, Artificial Sequence
  Synthetic Construct
AASTLQSGV

SEQ ID NO: 70 - protein, 13 aa, Artificial Sequence
  Synthetic Construct
YCQHYTSSPPVYT

SEQ ID NO: 71 - protein, 11 aa, Artificial Sequence
  Synthetic Construct
YCVQGLQTPWT

SEQ ID NO: 72 - protein, 12 aa, Artificial Sequence
  Synthetic Construct
YCQQYDQWPPWT

SEQ ID NO: 73 - protein, 11 aa, Artificial Sequence
  Synthetic Construct
YCQQSYSWPPT

SEQ ID NO: 74 - protein, 11 aa, Artificial Sequence
  Synthetic Construct
YCQQSYPTPLT

SEQ ID NO: 75 - protein, 11 aa, Artificial Sequence
  Synthetic Construct
YCQQSSSWPPT

SEQ ID NO: 76 - protein, 12 aa, Artificial Sequence
  Synthetic Construct
YCQQSYYWPPWT

SEQ ID NO: 77 - protein, 11 aa, Artificial Sequence
  Synthetic Construct
YCQHHYGTPLT

SEQ ID NO: 78 - protein, 13 aa, Artificial Sequence
  Synthetic Construct
YCQQYVSSPPEYT

SEQ ID NO: 79 - protein, 11 aa, Artificial Sequence
  Synthetic Construct
YCQQRDSWPYT

SEQ ID NO: 80 - protein, 11 aa, Artificial Sequence
  Synthetic Construct
YCEQSLEVPFT

SEQ ID NO: 81 - protein, 11 aa, Artificial Sequence
  Synthetic Construct
YCVQALQLPLT

SEQ ID NO: 82 - protein, 124 aa, Artificial Sequence
  Synthetic Construct
EVQLVESGGGLVQPGGSLRLSCAASGFTFSDYAIHWVRQAPGKGLEWIGIISPSSGSTNYAQKFQGRVTISRDNSKNTLYLQLNSLRAEDTAVYYCARDIHSGSSGYYYGFDVWGQGTLVTVSS

SEQ ID NO: 83 - protein, 118 aa, Artificial Sequence
  Synthetic Construct
EVQLVESGGGLVQPGGSLRLSCAASGYSITSGYYWAWIRQAPGKGLEWVSSISGSGSTTYYADSVKGRFTISRDNSKNTLYLQLNSLRAEDTAVYYCARDGFGYFDYWGQGTLVTVSS

SEQ ID NO: 84 - protein, 123 aa, Artificial Sequence
  Synthetic Construct
EVQLVESGGGLVQPGGSLRLSCAASGFTFSDYGIHWVRQAPGKGLEWIGEIYHSGSTYYSPSLKSRVTISRDNSKNTLYLQLNSLRAEDTAVYYCARDVAPGSSGYYDGFDFWGQGTLVTVSS

SEQ ID NO: 85 - protein, 121 aa, Artificial Sequence
  Synthetic Construct
EVQLVESGGGLVQPGGSLRLSCAASGYSISSGYHWDWIRQAPGKGLEWVSGISGYGGSTYYADSVKGRFTISRDNSKNTLYLQLNSLRAEDTAVYYCARHSYYGSGNFDYWGQGTLVTVSS

SEQ ID NO: 86 - protein, 116 aa, Artificial Sequence
  Synthetic Construct
EVQLVESGGGLVQPGGSLRLSCAASGFTFSDYWIHWVRQAPGKGLEWIGWISPSGGGTKYAQKFQGRVTISRDNSKNTLYLQLNSLRAEDTAVYYCARGAYEFDYWGQGTLVTVSS

SEQ ID NO: 87 - protein, 116 aa, Artificial Sequence
  Synthetic Construct
EVQLVESGGGLVQPGGSLRLSCAASGYSISSGYHWSWIRQAPGKGLEWLARIDWDDDKYYSTSLKSRLTISRDNSKNTLYLQLNSLRAEDTAVYYCARSYVYFDYWGQGTLVTVSS

SEQ ID NO: 88 - protein, 120 aa, Artificial Sequence
  Synthetic Construct
EVQLVESGGGLVQPGGSLRLSCAASGFSLSTGGVAVSWIRQAPGKGLEWIGEIYHSGSTYYSPSLKSRVTISRDNSKNTLYLQLNSLRAEDTAVYYCARRIATATYFDYWGQGTLVTVSS

SEQ ID NO: 89 - protein, 121 aa, Artificial Sequence
  Synthetic Construct
EVQLVESGGGLVQPGGSLRLSCAASGFSLSTGGVAVGWIRQAPGKGLEWVSAISGYGSTTYYADSVKGRFTISRDNSKNTLYLQLNSLRAEDTAVYYCARLPYSAYAFDYWGQGTLVTVSS

SEQ ID NO: 90 - protein, 114 aa, Artificial Sequence
  Synthetic Construct
EVQLVESGGGLVQPGGSLRLSCAASGFTFSGYAIHWVRQAPGKGLEWIGIISPSGGGTKYAQKFQGRVTISRDNSKNTLYLQLNSLRAEDTAVYYCARHPFAYWGQGTLVTVSS

SEQ ID NO: 91 - protein, 115 aa, Artificial Sequence
  Synthetic Construct
EVQLVESGGGLVQPGGSLRLSCAASGYTFSGYGIHWVRQAPGKGLEWIGEIYHSGSTYYSPSLKSRVTISRDNSKNTLYLQLNSLRAEDTAVYYCARRIDAFDIWGQGTLVTVSS

SEQ ID NO: 92 - protein, 115 aa, Artificial Sequence
  Synthetic Construct
EVQLVESGGGLVQPGGSLRLSCAASGYTFSGYAIHWVRQAPGKGLEWIGIISPSGGGTKYAQKFQGRVTISRDNSKNTLYLQLNSLRAEDTAVYYCARLYDVAYWGQGTLVTVSS

SEQ ID NO: 93 - protein, 117 aa, Artificial Sequence
  Synthetic Construct
EVQLVESGGGLVQPGGSLRLSCAASGFTFSDYAIHWVRQAPGKGLEWIGIISPSGGSTKYAQKFQGRVTISRDNSKNTLYLQLNSLRAEDTAVYYCARLGYGYFDVWGQGTLVTVSS

SEQ ID NO: 94 - protein, 118 aa, Artificial Sequence
  Synthetic Construct
EVQLVESGGGLVQPGGSLRLSCAASGYSITSGHYWSWIRQAPGKGLEWIGDISHSGSTYYSQSLKSRVTISRDNSKNTLYLQLNSLRAEDTAVYYCARGSRTGYFDYWGQGTLVTVSS

SEQ ID NO: 95 - protein, 114 aa, Artificial Sequence
  Synthetic Construct
DIQLTQSPSSLSASVGDRVTITCRASESVDFFGISFLAWYQQKPGKAPKLLIYDASNRATGIPSRFSGSGSGTDFTLTISSLQPEDFATYYCQHYTSSPPVYTFGQGTKVEIKR

SEQ ID NO: 96 - protein, 107 aa, Artificial Sequence
  Synthetic Construct
DIQLTQSPSSLSASVGDRVTITCSASSSVSYVYWYQQKPGKAPKLLIYDASSLESGVPSRFSGSGSGTDFTLTISSLQPEDFATYYCVQGLQTPWTFGQGTKVEIKR

SEQ ID NO: 97 - protein, 109 aa, Artificial Sequence
  Synthetic Construct
DIQLTQSPSSLSASVGDRVTITCRASQGIGSSLAWYQQKPGKAPKLLIYDASNRATGIPSRFSGSGSGTDFTLTISSLQPEDFATYYCQQYDQWPPWTFGQGTKVEIKR

SEQ ID NO: 98 - protein, 112 aa, Artificial Sequence
  Synthetic Construct
DIQLTQSPSSLSASVGDRVTITCRASESVDFFGKSFLHWYQQKPGKAPKLLIYDASNLETGVPSRFSGSGSGTDFTLTISSLQPEDFATYYCQQSYSWPPTFGQGTKVEIKR

SEQ ID NO: 99 - protein, 109 aa, Artificial Sequence
  Synthetic Construct
DIQLTQSPSSLSASVGDRVTITCRASQSVSSRFLAWYQQKPGKAPKLLIYDASNRATGIPSRFSGSGSGTDFTLTISSLQPEDFATYYCQQSYPTPLTFGQGTKVEIKR

SEQ ID NO: 100 - protein, 109 aa, Artificial Sequence
  Synthetic Construct
DIQLTQSPSSLSASVGDRVTITCRASQSVRGRFLAWYQQKPGKAPKLLIYDASNRATGIPSRFSGSGSGTDFTLTISSLQPEDFATYYCQQSSSWPPTFGQGTKVEIKR

SEQ ID NO: 101 - protein, 110 aa, Artificial Sequence
  Synthetic Construct
DIQLTQSPSSLSASVGDRVTITCRASQTVFSRYLAWYQQKPGKAPKLLIYDASNRATGIPSRFSGSGSGTDFTLTISSLQPEDFATYYCQQSYYWPPWTFGQGTKVEIKR

SEQ ID NO: 102 - protein, 108 aa, Artificial Sequence
  Synthetic Construct
DIQLTQSPSSLSASVGDRVTITCRASQGVSSYLAWYQQKPGKAPKLLIYAASTLQSGVPSRFSGSGSGTDFTLTISSLQPEDFATYYCQHHYGTPLTFGQGTKVEIKR

SEQ ID NO: 103 - protein, 114 aa, Artificial Sequence
  Synthetic Construct
DIQLTQSPSSLSASVGDRVTITCRASQSVDFYGISFLDWYQQKPGKAPKLLIYDASNRATGIPSRFSGSGSGTDFTLTISSLQPEDFATYYCQQYVSSPPEYTFGQGTKVEIKR

SEQ ID NO: 104 - protein, 112 aa, Artificial Sequence
  Synthetic Construct
DIQLTQSPSSLSASVGDRVTITCRASQSVDFDGFSFLHWYQQKPGKAPKLLIYDASSLESGVPSRFSGSGSGTDFTLTISSLQPEDFATYYCQQRDSWPYTFGQGTKVEIKR

SEQ ID NO: 105 - protein, 112 aa, Artificial Sequence
  Synthetic Construct
DIQLTQSPSSLSASVGDRVTITCRASQSVDFHGKSFLHWYQQKPGKAPKLLIYDASSLESGVPSRFSGSGSGTDFTLTISSLQPEDFATYYCEQSLEVPFTFGQGTKVEIKR

SEQ ID NO: 106 - protein, 112 aa, Artificial Sequence
  Synthetic Construct
DIQLTQSPSSLSASVGDRVTITCRASQSVDFYGISFLHWYQQKPGKAPKLLIYDASSLESGVPSRFSGSGSGTDFTLTISSLQPEDFATYYCVQALQLPLTFGQGTKVEIKR

SEQ ID NO: 107 - protein, 108 aa, Artificial Sequence
  Synthetic Construct
DIQLTQSPSSLSASVGDRVTITCRASQSISSYLNWYQQKPGKAPKLLIYDASNLETGVPSRFSGSGSGTDFTLTISSLQPEDFATYYCQHHYGTPLTFGQGTKVEIKR

SEQ ID NO: 108 - protein, 120 aa, Artificial Sequence
  Synthetic Construct
KAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPE

SEQ ID NO: 109-124 - skipped (16 entries)

SEQ ID NO: 125 - protein, 445 aa, Artificial Sequence
  Synthetic Construct
EVQLVESGGGLVQPGGSLRLSCAASGYSISSGYHWSWIRQAPGKGLEWLARIDWDDDKYYSTSLKSRLTISRDNSKNTLYLQLNSLRAEDTAVYYCARSYVYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 126 - protein, 446 aa, Artificial Sequence
  Synthetic Construct
EVQLVESGGGLVQPGGSLRLSCAASGYSISSGYHWSWIRQAPGKGLEWLARIDWDDDKYYSTSLKSRLTISRDNSKNTLYLQLNSLRAEDTAVYYCARSYVYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 127 - protein, 215 aa, Artificial Sequence
  Synthetic Construct
DIQLTQSPSSLSASVGDRVTITCRASQSVRGRFLAWYQQKPGKAPKLLIYDASNRATGIPSRFSGSGSGTDFTLTISSLQPEDFATYYCQQSSSWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 128 - protein, 446 aa, Artificial Sequence
  Synthetic Construct
EVQLVESGGGLVQPGGSLRLSCAASGFTFSDYAIHWVRQAPGKGLEWIGIISPSGGSTKYAQKFQGRVTISRDNSKNTLYLQLNSLRAEDTAVYYCARLGYGYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 129 - protein, 447 aa, Artificial Sequence
  Synthetic Construct
EVQLVESGGGLVQPGGSLRLSCAASGFTFSDYAIHWVRQAPGKGLEWIGIISPSGGSTKYAQKFQGRVTISRDNSKNTLYLQLNSLRAEDTAVYYCARLGYGYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 130 - protein, 218 aa, Artificial Sequence
  Synthetic Construct
DIQLTQSPSSLSASVGDRVTITCRASQSVDFYGISFLHWYQQKPGKAPKLLIYDASSLESGVPSRFSGSGSGTDFTLTISSLQPEDFATYYCVQALQLPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 131 - protein, 447 aa, Artificial Sequence
  Synthetic Construct
EVQLVESGGGLVQPGGSLRLSCAASGYSITSGYYWAWIRQAPGKGLEWVSSISGSGSTTYYADSVKGRFTISRDNSKNTLYLQLNSLRAEDTAVYYCARDGFGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 132 - protein, 213 aa, Artificial Sequence
  Synthetic Construct
DIQLTQSPSSLSASVGDRVTITCSASSSVSYVYWYQQKPGKAPKLLIYDASSLESGVPSRFSGSGSGTDFTLTISSLQPEDFATYYCVQGLQTPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 133 - protein, 448 aa, Artificial Sequence
  Synthetic Construct
EVQLVESGGGLVQPGGSLRLSCAASGYSITSGYYWAWIRQAPGKGLEWVSSISGSGSTTYYADSVKGRFTISRDNSKNTLYLQLNSLRAEDTAVYYCARDGFGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 134 - protein, 443 aa, Artificial Sequence
  Synthetic Construct
EVQLVESGGGLVQPGGSLRLSCAASGFTFSGYAIHWVRQAPGKGLEWIGIISPSGGGTKYAQKFQGRVTISRDNSKNTLYLQLNSLRAEDTAVYYCARHPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 135 - protein, 444 aa, Artificial Sequence
  Synthetic Construct
EVQLVESGGGLVQPGGSLRLSCAASGFTFSGYAIHWVRQAPGKGLEWIGIISPSGGGTKYAQKFQGRVTISRDNSKNTLYLQLNSLRAEDTAVYYCARHPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 136 - protein, 220 aa, Artificial Sequence
  Synthetic Construct
DIQLTQSPSSLSASVGDRVTITCRASQSVDFYGISFLDWYQQKPGKAPKLLIYDASNRATGIPSRFSGSGSGTDFTLTISSLQPEDFATYYCQQYVSSPPEYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 137 - protein, 444 aa, Artificial Sequence
  Synthetic Construct
EVQLVESGGGLVQPGGSLRLSCAASGYTFSGYAIHWVRQAPGKGLEWIGIISPSGGGTKYAQKFQGRVTISRDNSKNTLYLQLNSLRAEDTAVYYCARLYDVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 138 - protein, 445 aa, Artificial Sequence
  Synthetic Construct
EVQLVESGGGLVQPGGSLRLSCAASGYTFSGYAIHWVRQAPGKGLEWIGIISPSGGGTKYAQKFQGRVTISRDNSKNTLYLQLNSLRAEDTAVYYCARLYDVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 139 - protein, 218 aa, Artificial Sequence
  Synthetic Construct
DIQLTQSPSSLSASVGDRVTITCRASQSVDFHGKSFLHWYQQKPGKAPKLLIYDASSLESGVPSRFSGSGSGTDFTLTISSLQPEDFATYYCEQSLEVPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2024/0209091 A1
(43) Pub. Date: Jun. 27, 2024

(54) METHODS FOR TREATING CANCER USING ANTI-CTLA4 ANTIBODIES

(71) Applicant: ADAGENE PTE. LTD, Singapore (SG)

(72) Inventors: Peter Peizhi LUO, Morgan Hill, CA (US); Guizhong LIU, Suzhou (CN); Hua Chang GONG, Suzhou (CN); Songmao ZHENG, Suzhou (CN)

(73) Assignee: ADAGENE PTE. LTD, Singapore (SG)

(21) Appl. No.: 18/553,043

(22) PCT Filed: Mar. 28, 2022
(86) PCT No.: PCT/US2022/022198

§ 371 Date: Sep. 28, 2023

Related U.S. Application Data
(60) Provisional application No. 63/167,111, filed on Mar. 28, 2021.

(51) Int. Cl. C07K 16/28; A61P 35/04; G01N 33/569

(52) U.S. Cl. CPC C07K 16/2818; A61P 35/04; G01N 33/56972; C07K 2317/21; C07K 2317/24; C07K 2317/92; C07K 2317/94; G01N 2333/705

(57) ABSTRACT

Through the hybridoma technique, we got two hybridoma cells secreting anti-CTLA-4 mAb with high affinity, which were named 22G11 and 16C11 respectively. To construct the chimeric antibody light chain and heavy chain expression vectors, the cloned anti-CTL4 antibody's light chain (VL) and heavy chain variable region cDNAs (VH) were respectively fused to the human antibody light and heavy chain constant region genes (CH). Then appropriate light and heavy chain expression vectors were co-transfected into CHO-K1 cells and the clones producing the highest amount of chimeric antibodies were selected. In the end, the purity of the two chimeric antibodies was more than ninety percent. Both of them can bind Jurkat cells specifically as did the commercial anti-CTLA-4 mAb. Western blotting analysis showed that mAbs 22G11 and 16C11 reacted with one specific band with a molecular weight of about 22 kD, further indicating that 22G11 and 16C11 specifically recognized CTLA-4 molecule. Antigen-binding activity assay demonstrated that both of the two mAbs were shown to bind well to Jurkat cells, indicating that the two chimeric antibodies were correctly constructed and produced. In the competitive binding assay, c22G11 and c16C11 could effectively compete with their respective parental mouse antibodies for binding to Jurkat cells. The avidity (mean IC₅₀±SD) of c22G11 and c16C11 was equal to that of 22G11 and 16C11 respectively, which indicated that the chimeric antibodies possessed affinity and specificity similar to those of the original murine antibodies.

### CROSS-REFERENCE TO RELATED APPLICATIONS

This application claims priority to U.S. Provisional Patent Application No. 63/167,111, filed Mar. 28, 2021, the disclosure which is hereby incorporated herein by reference in its entirety for all purposes.

### FIELD OF THE INVENTION

The present application is in the field of cancer therapeutics, and relates to compositions and methods for treating cancers using antibodies that bind to human CTLA4.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 695402001340SEQLIST.TXT, date recorded: Mar. 25, 2022, size 98,612 bytes).

### BACKGROUND

CTLA4 is a member of the immunoglobulin (Ig) superfamily of proteins that acts to downregulate T-cell activation and maintain immunogenic homeostasis. It has been shown that in vivo antibody-mediated blockade of CTLA4 enhanced anti-cancer immune responses in a syngeneic murine prostate cancer model (Kwon et al. (1997) Proc Natl Acad Sci USA, 94(15):8099-103). In addition, blockade of CTLA4 function was shown to enhance anti-tumor T cell responses at various stages of tumor growth in tumor-bearing mice (Yang et al. (1997) Cancer Res 57(18):4036-41; Hurwitz et al. (1998) Proc Natl Acad Sci USA 95 (17):10067-7). However, the development of antibody-based therapeutics suitable for human use remains difficult, as translation from pre-clinical animal models to human safety is often poor. Accordingly, a need exists for anti-CTLA4 antibodies that are cross-reactive among different species, such as humans and experimental animals (e.g., mouse, monkey, rat, etc.), to concurrently enable animal model studies and provide suitable human therapeutic candidates. In addition, a need exists for the development of safer anti-CTLA4 antibodies that are only active in certain contexts, such as in the protease-rich tumor microenvironment.

### BRIEF SUMMARY

The present application provides methods for treating cancer with an anti-CTLA4 antibody, and methods of using biomarkers (e.g., pharmacodynamic biomarkers) for determining responsiveness of a patient to an anti-CTLA4 antibody treatment, for adjusting dosage and dosing schedule of the treatment, and for monitoring prognosis of the treatment.

Accordingly, in one aspect, provided herein is a method of treating a cancer in a subject, comprising: (a) administering to the subject an effective amount of an anti-CTLA4 antibody, wherein the antibody specifically binds to an epitope comprising amino acid residues Y105 and L106 of human CTLA4 but does not comprise residue I108, wherein the numbering of the amino acid residues is according to SEQ ID NO: 108, (b) subsequently determining a level of one or more biomarkers selected from the group consisting of IL-1β, IL-2, IL-6, IL-10, interferon (IFN)-γ, tumor necrosis factor (TNF)-α, soluble CTLA4 (sCTLA4), soluble PD-L1 (sPD-L1), soluble CD25 (sCD25), CXCL11, FoxP3, Ki67, CD8+ T cells, CD4+ T cells, CD8+ effector memory T (Tₑₘ) cells, CD4+ Tₑₘ cells, regulatory T (T_{reg}) cells, a ratio of CD8+ Tₑₘ cells to T_{reg} cells, a ratio of CD4+ Tₑₘ cells to T_{reg} cells, NK cells and B cells in a sample of the subject. In some embodiments, an increase or decrease of the one or more biomarkers after administration of the anti-CTLA4 antibody compared to the baseline level of the one or more biomarkers indicates an increased likelihood that the subject has an effective response to the CTLA4 antibody. In some embodiments, wherein the sample has an increase or decrease of the one or more biomarkers after administration of the anti-CTLA4 antibody compared to the baseline level of the one or more biomarkers, the method further comprises administering to the subject a further cycle of an effective amount of the anti-CTLA4 antibody.

In another aspect, provided herein is a method of providing a prognosis for a subject who has been administered with an effective amount of an anti-CTLA4 antibody, wherein the antibody specifically binds to an epitope comprising amino acid residues Y105 and L106 of human CTLA4 but does not comprise residue I108, wherein the numbering of the amino acid residues is according to SEQ ID NO: 108; the method comprising determining a level of one or more biomarkers selected from the group consisting of IL-1β, IL-2, IL-6, IL-10, IFN-γ, TNF-α, sCTLA4, sPD-L1, sCD25, CXCL11, FoxP3, Ki67, CD8+ T cells, CD4+ T cells, CD8+ Tₑₘ cells, CD4+ Tₑₘ cells, T_{reg} cells, a ratio of CD8+ Tₑₘ cells to T_{reg} cells, a ratio of CD4+ Tₑₘ cells to T_{reg} cells, NK cells and B cells in a sample of the subject, wherein an increase or decrease of the one or more biomarkers after administration of the anti-CTLA4 antibody compared to the baseline level of the one or more biomarkers indicates an increased likelihood that the subject has an effective response to the CTLA4 antibody.

In some embodiments according to any one of the methods described above, the one or more biomarkers comprise one or more biomarkers selected from the group consisting of CD8+ T cells, CD4+ T cells, CD8+ Tₑₘ cells, CD4+ Tₑₘ cells, T_{reg} cells, a ratio of CD8+ Tₑₘ cells to T_{reg} cells, a ratio of CD4+ Tₑₘ cells to T_{reg} cells, NK cells and B cells. In some embodiments, an increase in the level of CD8+ T cells, CD4+ T cells, CD8+ T_{reg} cells, CD4+ Tₑₘ cells, a ratio of CD8+ T_{reg} cells to T_{reg} cells, a ratio of CD4+ Tₑₘ cells to T_{reg} cells, NK cells and B cells after administration of the anti-CTLA4 antibody compared to the baseline level of the one or more biomarkers indicates an increased likelihood that the subject has an effective response to the CTLA4 antibody. In some embodiments, the one or more biomarkers comprise CD8+ Tₑₘ cells. In some embodiments, the one or more biomarkers comprise CD4+ Tₑₘ cells. In some embodiments, the one or more biomarkers comprise NK cells. In some embodiments, the one or more biomarkers comprise a ratio of CD8+ Tₑₘ cells to T_{reg} cells. In some embodiments, the one or more biomarkers comprise a ratio of CD4+ Tₑₘ cells to T_{reg} cells. In some embodiments, the one or more biomarkers comprise T_{reg} cells. In some embodiments, a decrease in the level of T_{reg} cells after administration of the anti-CTLA4 antibody compared to the baseline level of T_{reg} cells indicates an increased likelihood that the subject has an effective response to the CTLA4 antibody.

In some embodiments according to any one of the methods described above, the sample is a blood sample. In some embodiments, the sample is a tumor biopsy sample.

In some embodiments according to any one of the methods described above, the cancer is resistant or refractory to a prior therapy, wherein the prior therapy is an inhibitor of CTLA4, PD-1, or a PD-1 ligand. In some embodiments, the subject is resistant to or has relapsed from a prior therapy, wherein the prior therapy is an inhibitor of CTLA4, PD-1, or a PD-1 ligand. In some embodiments, the prior therapy is an inhibitor of CTLA4, such as ipilimumab. In some embodiments, the prior therapy is an inhibitor of PD-1, such as an anti-PD-1 antibody, for example, pembrolizumab. In some embodiments, the prior therapy is an inhibitor of a PD-1 ligand (e.g., PD-L1), for example an anti-PD-L1 antibody. In some embodiments, the prior therapy includes both an inhibitor of CTLA4 and am inhibitor of PD-1. In some embodiments, the prior therapy includes both an inhibitor of CTLA4 and am inhibitor of PD-L1. In some of the foregoing embodiments, the anti-CTLA4 antibody comprises a heavy chain variable region comprising the amino acid sequence of SEQ ID NO: 87 or an amino acid sequence having at least 90% sequence identity to the amino acid sequence of SEQ ID NO: 87, and/or a light chain variable region comprising the amino acid sequence of SEQ ID NO: 100 or an amino acid sequence having at least 90% sequence identity to the amino acid sequence of SEQ ID NO: 100. In some of the foregoing embodiments, the anti-CTLA4 antibody comprises a human IgG1 Fc region, such as a wildtype IgG1 Fc region or a variant that has enhanced ADCC activity. In some of the foregoing embodiments, the anti-CTLA4 antibody comprises a heavy chain comprising the amino acid sequence of SEQ ID NO: 125 and a light chain comprising the amino acid sequence of SEQ ID NO: 127. In some of the foregoing embodiments, the anti-CTLA4 antibody comprises a heavy chain comprising the amino acid sequence of SEQ ID NO: 126 and a light chain comprising the amino acid sequence of SEQ ID NO: 127. In some of the foregoing embodiments, the anti-CTLA4 antibody is TY21580.

Another aspect of the present application provides a method of treating a cancer in a subject, comprising administering to the subject an effective amount of an anti-CTLA4 antibody, wherein the antibody specifically binds to an epitope comprising amino acid residues Y105 and L106 of human CTLA4 but does not comprise residue I108, wherein the numbering of the amino acid residues is according to SEQ ID NO: 108, and wherein the cancer is resistant or refractory to a prior therapy, wherein the prior therapy is an inhibitor of CTLA4, PD-1, or a PD-1 ligand. In some embodiments, the present application provides a method of treating a cancer in a subject, comprising administering to the subject an effective amount of an anti-CTLA4 antibody, wherein the antibody specifically binds to an epitope comprising amino acid residues Y105 and L106 of human CTLA4 but does not comprise residue I108, wherein the numbering of the amino acid residues is according to SEQ ID NO: 108, and wherein the subject is resistant to or has relapsed from a prior therapy, wherein the prior therapy is an inhibitor of CTLA4, PD-1, or a PD-1 ligand. In some embodiments, the prior therapy is an inhibitor of CTLA4, such as ipilimumab. In some embodiments, the prior therapy is an inhibitor of PD-1, such as an anti-PD-1 antibody, for example, pembrolizumab. In some embodiments, the prior therapy is an inhibitor of a PD-1 ligand (e.g., PD-L1), for example an anti-PD-L1 antibody.

In some embodiments according to any one of the methods described above, the cancer is liver cancer, a cancer of the digestive system (e.g., colon cancer, colorectal cancer), lung cancer, bone cancer, heart cancer, brain cancer, kidney cancer, bladder cancer, a hematological cancer (e.g., leukemia), skin cancer, breast cancer, thyroid cancer, pancreatic cancer, a head and/or neck cancer, an eye-related cancer, a male reproductive system cancer (e.g., prostate cancer, testicular cancer), or a female reproductive system cancer (e.g., uterine cancer, cervical cancer). In some embodiments, the cancer is a solid cancer. In some embodiments, the cancer is urothelial carcinoma. In some embodiments, the cancer is advanced-stage cancer. In some embodiments, the cancer is metastatic cancer.

In some embodiments according to any one of the methods described above, the anti-CTLA4 antibody is administered at a dose of about 0.001 mg/kg to about 20 mg/kg. In other embodiments according to any one of the methods described above, the anti-CTLA4 antibody is administered at a dose of about 0.001 mg/kg to about 10 mg/kg. In some embodiments, the anti-CTLA4 antibody is administered at a dose of at least about 0.03 mg/kg. In some embodiments, the anti-CTLA4 antibody is administered at a dose of at least about 6 mg/kg. In some embodiments, the anti-CTLA4 antibody is administered at a dose of about 0.03 mg/kg, 0.1 mg/kg, 0.3 mg/kg, 1.0 mg/kg, 3.0 mg/kg, 6.0 mg/kg, 10.0 mg/kg, 15 mg/kg or 20 mg/kg. In some embodiments, the anti-CTLA4 antibody is administered intravenously. In some embodiments, the anti-CTLA4 antibody is administered subcutaneously. In some embodiments, the anti-CTLA4 antibody is administered about once every three weeks. In some embodiments, the subject receives at least 4 cycles of treatment with the anti-CTLA4 antibody. In some embodiments, the subject further receives a maintenance treatment comprising administering to the subject an effective amount of the anti-CTLA4 antibody about once every four weeks to about once every twelve weeks (e.g., once every 4, 6, 8, 10, or 12 weeks).

Another aspect of the present application provides a method of treating a cancer in a subject, comprising administering to the subject an effective amount of an anti-CTLA4 antibody, wherein the antibody specifically binds to an epitope comprising amino acid residues Y105 and L106 of human CTLA4 but does not comprise residue I108, wherein the numbering of the amino acid residues is according to SEQ ID NO: 108, and wherein the anti-CTLA4 antibody is administered at a dose of at least 6 mg/kg.

In some embodiments according to any one of the methods described above, the anti-CTLA4 antibody is administered at a dose of about 6 mg/kg (e.g., 6 mg/kg).

In some embodiments, the anti-CTLA4 antibody is administered at a dose of about 10 mg/kg (e.g., 10 mg/kg).

In some embodiments, the anti-CTLA4 antibody is administered at a dose of about 3 mg/kg (e.g., 3 mg/kg) once every three weeks. In some embodiments, the anti-CTLA4 antibody is administered at a dose of about 6 mg/kg (e.g., 6 mg/kg) once every three weeks. In some embodiments, the anti-CTLA4 antibody is administered at a dose of about 10 mg/kg (e.g., 10 mg/kg) once every three weeks. In some embodiments, the anti-CTLA4 antibody is administered at a dose of about 10 mg/kg (e.g., 10 mg/kg) once every three weeks. In some embodiments, the anti-CTLA4 antibody is administered at a dose of about 15 mg/kg (e.g., 15 mg/kg) once every three weeks. In some embodiments, the anti-CTLA4 antibody is administered at a dose of about 20 mg/kg (e.g., 15 mg/kg) once every three weeks. In some embodiments, the cancer is urothelial carcinoma. In some of the foregoing embodiments, the anti-CTLA4 antibody comprises a heavy chain variable region comprising the amino acid sequence of SEQ ID NO: 87 or an amino acid sequence having at least 90% sequence identity to the amino acid sequence of SEQ ID NO: 87, and/or a light chain variable region comprising the amino acid sequence of SEQ ID NO: 100 or an amino acid sequence having at least 90% sequence identity to the amino acid sequence of SEQ ID NO: 100. In some of the foregoing embodiments, the anti-CTLA4 antibody comprises a human IgG1 Fc region, such as a wildtype IgG1 Fc region or a variant that has enhanced ADCC activity. In some of the foregoing embodiments, the anti-CTLA4 antibody comprises a heavy chain comprising the amino acid sequence of SEQ ID NO: 125 and a light chain comprising the amino acid sequence of SEQ ID NO: 127. In some of the foregoing embodiments, the anti-CTLA4 antibody comprises a heavy chain comprising the amino acid sequence of SEQ ID NO: 126 and a light chain comprising the amino acid sequence of SEQ ID NO: 127. In some of the foregoing embodiments, the anti-CTLA4 antibody is TY21580.

In some embodiments according to any one of the methods described above, the subject is human.

In some embodiments according to any one of the methods described above, the anti-CTLA4 antibody is cross-reactive with a CTLA4 polypeptide from at least one non-human animal selected from the group consisting of cynomolgus monkey, mouse, rat, and dog. In some embodiments, the antibody binds to cynomolgus monkey CTLA4 and mouse CTLA4. In some embodiments, the antibody binds to human CTLA4, cynomolgus monkey CTLA4, mouse CTLA4, rat CTLA4, and/or dog CTLA4 with a dissociation constant (K_{D}) of about 350 nM or less (e.g., about 300 nM or less, about 200 nM or less, about 100 nM or less, about 50 nM or less, or about 10 nM or less). In some embodiments, the K_{D} is measured by surface plasmon resonance (SPR). In some embodiments, binding of the antibody to CTLA4 induces antibody-dependent cell cytotoxicity (ADCC) against a CTLA4-expressing cell. In some embodiments, binding of the antibody to CTLA4 induces ADCC against a Treg cell. In some embodiments, binding of the anti-CTLA4 antibody described herein induces antibody-dependent cell cytotoxicity (ADCC) against a CTLA4-expressing human cell or a human Treg cell, wherein the ADCC activity of the anti-CTL4 antibody is higher than the ADCC activity of ipilimumab in vitro, and wherein both antibodies comprise wild type human IgG1 Fc region. In some embodiments, binding of the anti-CTLA4 antibody described herein induces antibody-dependent cell cytotoxicity (ADCC) against a CTLA4-expressing human cell or a human Treg cell, wherein the ADCC activity of the anti-CTLA4 antibody is two times or higher than the ADCC activity of ipilimumab in vitro, and wherein both antibodies comprise wild type human IgG1 Fc region. In some embodiments, the EC50 of the anti-CTL4 antibody ADCC activity is 50% or less than the EC50 of ipilimumab ADCC activity in vitro. In some embodiments, the anti-CTLA4 antibody depletes Treg cells selectively in tumor microenvironment (e.g., reducing percentage of Treg cells in tumor infiltrating lymphocytes), as compared to PBMC or spleen in a mouse cancer model.

In some embodiments according to any one of the methods described above, the antibody specifically binds to an epitope comprising amino acid residues at a ligand binding site of human CTLA4, such as CD80 and/or CD86 binding site of human CTLA4. In some embodiments, the antibody specifically binds to an epitope similar to a ligand binding site of human CTLA4, such as CD80 and/or CD86 binding site of human CTLA4. In some embodiments, the anti-CTLA4 antibody blocks binding of CD80 and/or CD86 to human CTLA4. In some embodiments, the anti-CTLA4 antibody has an IC50 higher than the IC50 of ipilimumab for blocking binding of CD80 and/or CD86 to human CTLA4. In some embodiments, the anti-CTLA4 antibody has an IC50 that is 3.5 times or higher (including 3.9 times or higher) than the IC50 of ipilimumab for blocking binding of CD80 and/or CD86 to human CTLA4 in an assay that CD86 or CD80 is plate bound and CTLA4 is in solution or CTLA4 displayed on cell surface.

In some embodiments according to any one of the methods described above, the antibody comprises a heavy chain variable region and a light chain variable region, a) wherein the heavy chain variable region comprises an HVR-H1, an HVR-H2, and an HVR-H3, wherein the HVR-H1 comprises an amino acid sequence according to a formula selected from the group consisting of: Formula (I): X1TFSX2YX3IHWV (SEQ ID NO: 1), wherein X1 is F or Y, X2 is D or G, and X3 is A, G, or W; Formula (II): YSIX1SGX2X3WX4WI (SEQ ID NO: 2), wherein X1 is S or T, X2 is H or Y, X3 is H or Y, and X4 is A, D, or S; and Formula (III): FSLSTGGVAVX1WI (SEQ ID NO: 3), wherein X1 is G or S; wherein the HVR-H2 comprises an amino acid sequence according to a formula selected from the group consisting of: Formula (IV): IGX1IX2HSGSTYYSX3SLKSRV (SEQ ID NO: 4), wherein X1 is D or E, X2 is S or Y, and X3 is P or Q; Formula (V): IGX1ISPSX2GX3TX4YAQKFQGRV (SEQ ID NO: 5), wherein X1 is I or W, X2 is G or S, X3 is G or S, and X4 is K or N; and Formula (VI): VSX1ISGX2GX3X4TYYADSVKGRF (SEQ ID NO: 6), wherein X1 is A, G, or S, X2 is S or Y, X3 is G or S, and X4 is S or T; and wherein the HVR-H3 comprises an amino acid sequence according to a formula selected from the group consisting of: Formula (VII): ARX1X2X3X4FDX5 (SEQ ID NO: 7), wherein X1 is G, R, or S, X2 is A, I, or Y, X3 is D, V, or Y, X4 is A, E, or Y, and X5 is I or Y; Formula (VIII): ARX1GX2GYFDX3 (SEQ ID NO: 8), wherein X1 is D or L, X2 is F or Y, and X3 is V or Y; Formula (IX): ARX1X2X3X4AX5X6FDY (SEQ ID NO: 9), wherein X1 is L or R, X2 is I or P, X3 is A or Y, X4 is S or T, X5 is T or Y, and X6 is A or Y; Formula (X): ARDX1X2X3GSSGYYX4GFDX5 (SEQ ID NO: 10), wherein X1 is I or V, X2 is A or H, X3 is P or S, X4 is D or Y, and X5 is F or V; and b) wherein the light chain variable region comprises an HVR-L1, an HVR-L2, and an HVR-L3, wherein the HVR-L1 comprises an amino acid sequence according to a formula selected form the group consisting of: Formula (XI): RASQX1X2X3SX4LX5 (SEQ ID NO: 11), wherein X1 is G or S, X2 is I or V, X3 is G or S, X4 is S or Y, and X5 is A or N; Formula (XII): RASQX1VX2X3RX4LA (SEQ ID NO: 12), wherein X1 is S or T, X2 is F, R, or S, X3 is G or S, and X4 is F or Y; and Formula (XIII): RASX1SVDFX2GX3SFLX4 (SEQ ID NO: 13), wherein X1 is E or Q, X2 is D, F, H, or Y, X3 is F, I, or K, and X4 is A, D, or H; wherein the HVR-L2 comprises an amino acid sequence according to Formula (XIV): X1ASX2X3X4X5GX6 (SEQ ID NO: 14), wherein X1 is A or D, X2 is N, S, or T, X3 is L or R, X4 is A, E, or Q, X5 is S or T, and X6 is I or V; and wherein the HVR-L3 comprises an amino acid sequence according to a formula selected from the group consisting of: Formula (XV): YCX1X2X3X4X5X6PX7T (SEQ ID NO: 15), wherein X1 is E, Q, or V, X2 is H or Q, X3 is A, G, H, R, or S, X4 is D, L, S, or Y, X5 is E, G, P, Q, or S, X6 is L, T, V, or W, and X7 is F, L, P, W, or Y; Formula (XVT): YCQQX1X2X3WPPWT (SEQ ID NO: 16), wherein X1 is S or Y, X2 is D or Y, and X3 is Q or Y; and Formula (XVII): YCQX1YX2SSPPX3YT (SEQ ID NO: 17), wherein X1 is H or Q, X2 is T or V, and X3 is E or V. In some embodiments, the HVR-H1 comprises an amino acid sequence selected from the group consisting of SEQ ID NOS: 18-29, the HVR-H2 comprises an amino acid sequence selected from the group consisting of SEQ ID NOS: 30-39, the HVR-H3 comprises an amino acid sequence selected from the group consisting of SEQ ID NOS: 40-52, the HVR-L1 comprises an amino acid sequence selected from the group consisting of SEQ ID NOS: 53-65, the HVR-L2 comprises an amino acid sequence selected from the group consisting of SEQ ID NOS: 66-69, and the HVR-L3 comprises an amino acid sequence selected from the group consisting of SEQ ID NOS: 70-81. In some embodiments, the antibody comprises: a) an HVR-H1 comprising the amino acid sequence of SEQ ID NO: 18, an HVR-H2 comprising the amino acid sequence of SEQ ID NO: 30, an HVR-H3 comprising the amino acid sequence of SEQ ID NO: 40, an HVR-L1 comprising the amino acid sequence of SEQ ID NO: 53, an HVR-L2 comprising the amino acid sequence of SEQ ID NO: 66, and an HVR-L3 comprising the amino acid sequence of SEQ ID NO: 70; b) an HVR-H1 comprising the amino acid sequence of SEQ ID NO: 19, an HVR-H2 comprising the amino acid sequence of SEQ ID NO: 31, an HVR-H3 comprising the amino acid sequence of SEQ ID NO: 41, an HVR-L1 comprising the amino acid sequence of SEQ ID NO: 54, an HVR-L2 comprising the amino acid sequence of SEQ ID NO: 67, and an HVR-L3 comprising the amino acid sequence of SEQ ID NO: 71; c) an HVR-H1 comprising the amino acid sequence of SEQ ID NO: 20, an HVR-H2 comprising the amino acid sequence of SEQ ID NO: 32, an HVR-H3 comprising the amino acid sequence of SEQ ID NO: 42, an HVR-L1 comprising the amino acid sequence of SEQ ID NO: 55, an HVR-L2 comprising the amino acid sequence of SEQ ID NO: 66, and an HVR-L3 comprising the amino acid sequence of SEQ ID NO: 72; d) an HVR-H1 comprising the amino acid sequence of SEQ ID NO: 21 an HVR-H2 comprising the amino acid sequence of SEQ ID NO: 33, an HVR-H3 comprising the amino acid sequence of SEQ ID NO: 43, an HVR-L1 comprising the amino acid sequence of SEQ ID NO: 56, an HVR-L2 comprising the amino acid sequence of SEQ ID NO: 68, and an HVR-L3 comprising the amino acid sequence of SEQ ID NO: 73; e) an HVR-H1 comprising the amino acid sequence of SEQ ID NO: 22, an HVR-H2 comprising the amino acid sequence of SEQ ID NO: 34, an HVR-H3 comprising the amino acid sequence of SEQ ID NO: 44, an HVR-L1 comprising the amino acid sequence of SEQ ID NO: 57, an HVR-L2 comprising the amino acid sequence of SEQ ID NO: 66, and an HVR-L3 comprising the amino acid sequence of SEQ ID NO: 74; f) an HVR-H1 comprising the amino acid sequence of SEQ ID NO: 23, an HVR-H2 comprising the amino acid sequence of SEQ ID NO: 35, an HVR-H3 comprising the amino acid sequence of SEQ ID NO: 45, an HVR-L1 comprising the amino acid sequence of SEQ ID NO: 58, an HVR-L2 comprising the amino acid sequence of SEQ ID NO: 66, and an HVR-L3 comprising the amino acid sequence of SEQ ID NO: 75; g) an HVR-H1 comprising the amino acid sequence of SEQ ID NO: 24, an HVR-H2 comprising the amino acid sequence of SEQ ID NO: 32, an HVR-H3 comprising the amino acid sequence of SEQ ID NO: 46, an HVR-L1 comprising the amino acid sequence of SEQ ID NO: 59, an HVR-L2 comprising the amino acid sequence of SEQ ID NO: 66, and an HVR-L3 comprising the amino acid sequence of SEQ ID NO: 76; h) an HVR-H1 comprising the amino acid sequence of SEQ ID NO: 25, an HVR-H2 comprising the amino acid sequence of SEQ ID NO: 36, an HVR-H3 comprising the amino acid sequence of SEQ ID NO: 47, an HVR-L1 comprising the amino acid sequence of SEQ ID NO: 60, an HVR-L2 comprising the amino acid sequence of SEQ ID NO: 69, and an HVR-L3 comprising the amino acid sequence of SEQ ID NO: 77; i) an HVR-H1 comprising the amino acid sequence of SEQ ID NO: 26, an HVR-H2 comprising the amino acid sequence of SEQ ID NO: 37, an HVR-H3 comprising the amino acid sequence of SEQ ID NO: 48, an HVR-L1 comprising the amino acid sequence of SEQ ID NO: 61, an HVR-L2 comprising the amino acid sequence of SEQ ID NO: 66, and an HVR-L3 comprising the amino acid sequence of SEQ ID NO: 78; j) an HVR-H1 comprising the amino acid sequence of SEQ ID NO: 27, an HVR-H2 comprising the amino acid sequence of SEQ ID NO: 32, an HVR-H3 comprising the amino acid sequence of SEQ ID NO: 49, an HVR-L1 comprising the amino acid sequence of SEQ ID NO: 62, an HVR-L2 comprising the amino acid sequence of SEQ ID NO: 67, and an HVR-L3 comprising the amino acid sequence of SEQ ID NO: 79; k) an HVR-H1 comprising the amino acid sequence of SEQ ID NO: 28, an HVR-H2 comprising the amino acid sequence of SEQ ID NO: 37, an HVR-H3 comprising the amino acid sequence of SEQ ID NO: 50, an HVR-L1 comprising the amino acid sequence of SEQ ID NO: 63, an HVR-L2 comprising the amino acid sequence of SEQ ID NO: 67, and an HVR-L3 comprising the amino acid sequence of SEQ ID NO: 80; l) an HVR-H1 comprising the amino acid sequence of SEQ ID NO: 18, an HVR-H2 comprising the amino acid sequence of SEQ ID NO: 38, an HVR-H3 comprising the amino acid sequence of SEQ ID NO: 51, an HVR-L1 comprising the amino acid sequence of SEQ ID NO: 64, an HVR-L2 comprising the amino acid sequence of SEQ ID NO: 67, and an HVR-L3 comprising the amino acid sequence of SEQ ID NO: 81; or m) an HVR-H1 comprising the amino acid sequence of SEQ ID NO: 29, an HVR-H2 comprising the amino acid sequence of SEQ ID NO: 39, an HVR-H3 comprising the amino acid sequence of SEQ ID NO: 52, an HVR-L1 comprising the amino acid sequence of SEQ ID NO: 65, an HVR-L2 comprising the amino acid sequence of SEQ ID NO: 68, and an HVR-L3 comprising the amino acid sequence of SEQ ID NO: 77. In some embodiments, the heavy chain variable region comprises an amino acid sequence selected from the group consisting of SEQ ID NOS: 82-94, and/or the light chain variable region comprises an amino acid sequence selected from the group consisting of SEQ ID NOS: 95-107. In some embodiments, the antibody comprises: a) a heavy chain variable region comprising the amino acid sequence of SEQ ID NO: 82 or a variant thereof having at least about 90% (e.g., at least about 92%, 95%, 98%, 99% or more) sequence identity to the amino acid sequence of SEQ ID NO: 82, and a light chain variable region comprising the amino acid sequence of SEQ ID NO: 95 or a variant thereof having at least about 90% (e.g., at least about 92%, 95%, 98%, 99% or more) sequence identity to the amino acid sequence of SEQ ID NO: 95; b) a heavy chain variable region comprising the amino acid sequence of SEQ ID NO: 83 or a variant thereof having at least about 90% (e.g., at least about 92%, 95%, 98%, 99% or more) sequence identity to the amino acid sequence of SEQ ID NO: 83, and a light chain variable region comprising the amino acid sequence of SEQ ID NO: 96 or a variant thereof having at least about 90% (e.g., at least about 92%, 95%, 98%, 99% or more) sequence identity to the amino acid sequence of SEQ ID NO: 96; c) a heavy chain variable region comprising the amino acid sequence of SEQ ID NO: 84 or a variant thereof having at least about 90% (e.g., at least about 92%, 95%, 98%, 99% or more) sequence identity to the amino acid sequence of SEQ ID NO: 84, and a light chain variable region comprising the amino acid sequence of SEQ ID NO: 97 or a variant thereof having at least about 90% (e.g., at least about 92%, 95%, 98%, 99% or more) sequence identity to the amino acid sequence of SEQ ID NO: 97; d) a heavy chain variable region comprising the amino acid sequence of SEQ ID NO: 85 or a variant thereof having at least about 90% (e.g., at least about 92%, 95%, 98%, 99% or more) sequence identity to the amino acid sequence of SEQ ID NO: 85, and a light chain variable region comprising the amino acid sequence of SEQ ID NO: 98 or a variant thereof having at least about 90% (e.g., at least about 92%, 95%, 98%, 99% or more) sequence identity to the amino acid sequence of SEQ ID NO: 98; e) a heavy chain variable region comprising the amino acid sequence of SEQ ID NO: 86 or a variant thereof having at least about 90% (e.g., at least about 92%, 95%, 98%, 99% or more) sequence identity to the amino acid sequence of SEQ ID NO: 86, and a light chain variable region comprising the amino acid sequence of SEQ ID NO: 99 or a variant thereof having at least about 90% (e.g., at least about 92%, 95%, 98%, 99% or more) sequence identity to the amino acid sequence of SEQ ID NO: 99; f) a heavy chain variable region comprising the amino acid sequence of SEQ ID NO: 87 or a variant thereof having at least about 90% (e.g., at least about 92%, 95%, 98%, 99% or more) sequence identity to the amino acid sequence of SEQ ID NO: 87, and a light chain variable region comprising the amino acid sequence of SEQ ID NO: 100 or a variant thereof having at least about 90% (e.g., at least about 92%, 95%, 98%, 99% or more) sequence identity to the amino acid sequence of SEQ ID NO: 100; g) a heavy chain variable region comprising the amino acid sequence of SEQ ID NO: 88 or a variant thereof having at least about 90% (e.g., at least about 92%, 95%, 98%, 99% or more) sequence identity to the amino acid sequence of SEQ ID NO: 88, and a light chain variable region comprising the amino acid sequence of SEQ ID NO: 101 or a variant thereof having at least about 90% (e.g., at least about 92%, 95%, 98%, 99% or more) sequence identity to the amino acid sequence of SEQ ID NO: 101; h) a heavy chain variable region comprising the amino acid sequence of SEQ ID NO: 89 or a variant thereof having at least about 90% (e.g., at least about 92%, 95%, 98%, 99% or more) sequence identity to the amino acid sequence of SEQ ID NO: 89, and a light chain variable region comprising the amino acid sequence of SEQ ID NO: 102 or a variant thereof having at least about 90% (e.g., at least about 92%, 95%, 98%, 99% or more) sequence identity to the amino acid sequence of SEQ ID NO: 102; i) a heavy chain variable region comprising the amino acid sequence of SEQ ID NO: 90 or a variant thereof having at least about 90% (e.g., at least about 92%, 95%, 98%, 99% or more) sequence identity to the amino acid sequence of SEQ ID NO: 90, and a light chain variable region comprising the amino acid sequence of SEQ ID NO: 103 or a variant thereof having at least about 90% (e.g., at least about 92%, 95%, 98%, 99% or more) sequence identity to the amino acid sequence of SEQ ID NO: 103; j) a heavy chain variable region comprising the amino acid sequence of SEQ ID NO: 91 or a variant thereof having at least about 90% (e.g., at least about 92%, 95%, 98%, 99% or more) sequence identity to the amino acid sequence of SEQ ID NO: 91, and a light chain variable region comprising the amino acid sequence of SEQ ID NO: 104 or a variant thereof having at least about 90% (e.g., at least about 92%, 95%, 98%, 99% or more) sequence identity to the amino acid sequence of SEQ ID NO: 104; k) a heavy chain variable region comprising the amino acid sequence of SEQ ID NO: 92 or a variant thereof having at least about 90% (e.g., at least about 92%, 95%, 98%, 99% or more) sequence identity to the amino acid sequence of SEQ ID NO: 92, and a light chain variable region comprising the amino acid sequence of SEQ ID NO: 105 or a variant thereof having at least about 90% (e.g., at least about 92%, 95%, 98%, 99% or more) sequence identity to the amino acid sequence of SEQ ID NO: 105; l) a heavy chain variable region comprising the amino acid sequence of SEQ ID NO: 93 or a variant thereof having at least about 90% (e.g., at least about 92%, 95%, 98%, 99% or more) sequence identity to the amino acid sequence of SEQ ID NO: 93, and a light chain variable region comprising the amino acid sequence of SEQ ID NO: 106 or a variant thereof having at least about 90% (e.g., at least about 92%, 95%, 98%, 99% or more) sequence identity to the amino acid sequence of SEQ ID NO: 106; or m) a heavy chain variable region comprising the amino acid sequence of SEQ ID NO: 94 or a variant thereof having at least about 90% (e.g., at least about 92%, 95%, 98%, 99% or more) sequence identity to the amino acid sequence of SEQ ID NO: 94, and a light chain variable region comprising the amino acid sequence of SEQ ID NO: 107 or a variant thereof having at least about 90% (e.g., at least about 92%, 95%, 98%, 99% or more) sequence identity to the amino acid sequence of SEQ ID NO: 107.

In some embodiments according to any one of the methods described above, the anti-CTLA4 antibody described herein comprises a heavy chain variable region and a light chain variable region, wherein one, two, three, four, five, or six HVRs of the antibody comprise a HVR sequence shown in Table A. In some embodiments, the anti-CTLA4 antibody comprises a heave chain variable region comprising an HVR-H1, an HVR-H2, and an HVR-H3, wherein the HVR-H1 comprises the amino acid sequence of SEQ ID NO: 23, or the HVR-H2 comprises the amino acid sequence of SEQ ID NO: 35, or the HVR-H3 comprises the amino acid sequence of SEQ ID NO: 45. In some embodiments, the anti-CTLA4 antibody comprises a light chain variable region comprising an HVR-L1, an HVR-L2, and an HVR-L3, wherein the HVR-L1 comprises the amino acid sequence of SEQ ID NO: 58, or the HVR-L2 comprises the antibody comprises the amino acid sequence of SEQ ID NO: 66, or the HVR-L3 comprises the amino acid sequence of SEQ ID NO: 75. In some embodiments, the anti-CTLA4 antibody comprises (a) a heavy chain variable region comprising an HVR-H1 comprising the amino acid sequence of SEQ ID NO: 23, an HVR-H2 comprising the amino acid sequence of SEQ ID NO: 35, and an HVR-H3 comprising the amino acid sequence of SEQ ID NO: 45, and/or a light chain variable region comprising an HVR-L1 comprising the amino acid sequence of SEQ ID NO: 58, an HVR-L2 comprising the amino acid sequence of SEQ ID NO: 66, and an HVR-L3 comprising the amino acid sequence of SEQ ID NO: 75. In some embodiments, one, two, three, four, five or six of the HVRs of the antibody may comprise one, two or three conservative amino acid substitutions in the HVRs. In some embodiments, the anti-CTLA4 antibody comprises (b) a heavy chain variable region comprising the amino acid sequence of SEQ ID NO: 87 or an amino acid sequence having at least 90% (e.g., 91%, 92%, 93%, 95%, 96%, 97%, 98%, or 99%) sequence identity to the amino acid sequence of SEQ ID NO: 87, and/or a light chain variable region comprising the amino acid sequence of SEQ ID NO: 100 or an amino acid sequence having at least 90% (e.g., 91%, 92%, 93%, 95%, 96%, 97%, 98%, or 99%) sequence identity to the amino acid sequence of SEQ ID NO: 100.

In some embodiments according to anyone of the methods described above, the antibody is a human antibody. In some embodiments, the antibody comprises an IgG1, IgG2, IgG3, or IgG4 Fc region (such as human IgG1, IgG2, IgG3, or IgG4 Fc region). In some embodiments, the antibody comprising a human IgG1 or a variant that has enhanced ADCC activity. In some embodiments, the antibody comprises a human IgG1 with reduced fucosylation (or non-fucosylated).

In some embodiments according to any one of the methods described above, the method further comprises administering to the subject an effective amount of at least one additional therapeutic agent. In some embodiments, the at least one additional therapeutic agent is selected from the group consisting of viral gene therapy, immune checkpoint inhibitors, target therapies, radiation therapies, vaccination therapies, and chemotherapies. In some embodiments, the method comprises administering to the subject an effective amount of the anti-CTLA4 antibody described herein prior to a surgery or after a surgery to remove the tumor in the subject.

It is to be understood that one, some, or all of the properties of the various embodiments described above and herein may be combined to form other embodiments of the present application. These and other aspects of the present application will become apparent to one of skill in the art. These and other embodiments of the present application are further described by the detailed description that follows.

### BRIEF DESCRIPTION OF THE DRAWINGS

FIG. 1 shows the change in T cell levels relative to baseline in patients treated with the anti-CTLA4 antibody TY21580 over time. The x-axis shows the time of sampling, the y-axis shows the % change in absolute T cell counts per μL relative to baseline, and the lines represent values from different subjects.

FIG. 2 shows the change in NK cell levels relative to baseline in patients treated with TY21580 over time. The x-axis shows the time of sampling, the y-axis shows the % change in absolute NK cell counts per μL relative to baseline, and the lines represent values from different subjects.

FIG. 3 shows the percentage of T_{reg} cells out of the total CD4+ T cell population in patients treated with TY21580 over time. The x-axis shows the time of sampling, the y-axis shows the % change T_{reg} levels relative to baseline, and the lines represent values from different subjects.

FIG. 4 shows the change in T cell levels relative to baseline in patients treated with TY21580 in different dose cohorts of TY21580. The x-axis shows the dose, the y-axis shows the % change in absolute T cell counts per μL relative to baseline, and the lines represent values from different subjects.

FIG. 5 shows the change in NK cell levels relative to baseline in patients treated with TY21580 in different dose cohorts of TY21580. The x-axis shows the dose, the y-axis shows the % change in absolute NK cell counts per μL relative to baseline, and the lines represent values from different subjects.

FIG. 6 shows the percentage of T_{reg} cells out of the total CD4+ T cell population in patients treated with TY21580 in different dose cohorts of TY21580. The x-axis shows the dose, the y-axis shows the % change in T_{reg} levels relative to baseline, and the lines represent values from different subjects.

FIG. 7 shows the percentage of effector memory (EM) CD8+ T cells out of the total CD8+ T cell population in patients treated with TY21580 in different dose cohorts of TY21580. The x-axis shows the dose, the y-axis shows the % change in EM CD8+ T cell levels relative to baseline, and the lines represent values from different subjects.

FIG. 8 shows the percentage of EM CD4⁺ T cells out of the total CD4⁺ T cell population in patients treated with TY21580 in different dose cohorts of TY21580. The x-axis shows the dose, the y-axis shows the % change in EM CD4⁺ T cell levels relative to baseline, and the lines represent values from different subjects.

FIGS. 9A-9C show the results of lymphocyte profiling of three subjects at either a 0.1 mg/kg or 0.3 mg/kg dose of TY21580. FIG. 9A shows (from left to right) the % change in CD8⁺ T_{EM}/Treg ratio, the % change in CD8⁺ T_{EM} cells, and the % change in T_{reg} cells for subject 6102-003 at a 0.1 mg/kg dose. FIG. 9B shows (from left to right) the % change in CD8⁺ T_{EM}/Treg ratio, the % change in CD8⁺ T_{EM} cells, and the % change in T_{reg} cells for subject 6101-004 at a 0.1 mg/kg dose. FIG. 9C shows (from left to right) the % change in CD8⁺ T_{EM}/Treg ratio, the % change in CD8⁺ T_{EM} cells, and the % change in T_{reg} cells for subject 6101-005 at a 0.3 mg/kg dose. In each plot, the x-axis shows the time of sampling, and the y-axis shows the percentage change relative to baseline.

FIG. 10 shows the results of lymphocyte profiling subject 6102-002 at a 0.03 mg/kg dose of TY21580. The plots show (from left to right on the top row) the % change in CD8⁺ T_{EM}/T_{reg} ratio, the % change in CD8⁺ T_{EM} cells, and the % change in T_{reg} cells, and (from left to right on the bottom row) the % change in CD4⁺ T_{EM} cells, the % change in NK cells, and the % change in B cells. In each plot, the x-axis shows the time of sampling, and the y-axis shows the percentage change relative to baseline.

FIG. 11 shows the results of population pharmacokinetic modeling using a 2-compartment model for TY21580 in the phase 1 study.

FIGS. 12A-12B show the results of population pharmacokinetic modeling for the phase 1 study. FIG. 12A shows goodness-of-fit plots. FIG. 12B shows a diagnostic plot.

FIG. 13 shows the % change in CD8⁺ T_{EM}/T_{reg} cell ratio in patients treated with TY21580 in different dose cohorts of TY21580. The x-axis shows the dose, the y-axis shows the percentage, and the lines represent values from different subjects.

FIG. 14 shows the % change in CD4⁺ T_{EM}/T_{reg} cell ratio inpatients treated with TY21580 in different dose cohorts of TY21580. The x-axis shows the dose, the y-axis shows the percentage, and the lines represent values from different subjects.

FIGS. 15A-15D show information regarding the ongoing study. FIG. 15A describes the dosing schedule of the study. FIG. 15B shows information regarding the subjects of the ongoing study and provides information regarding the amount and duration of TY21580 dosing for each. FIG. 15C shows the increase in CD8+ T cells in Subject #23 between C1D1 predose and C2D1. FIG. 15D shows treatment-related adverse events patients of the study have experienced.

FIG. 16 shows the serum pharmacokinetics of TY21580. The x-axis shows the day after dose, the y-axis shows the measured drug concentration in patient serum, and the lines represent different doses.

FIGS. 17A-17C show kinetics of serum IFN-γ levels in patients treated with TY21580. FIG. 17A shows the change in relative abundance of IFN-γ to baseline in patients treated with TY21580. The x-axis shows the dose cohorts, the y-axis shows the percent change, and the lines represent values from different subjects. FIG. 17B shows the change in relative abundance of IFN-γ between baseline and C1D2 in patients treated with TY21580. The x-axis shows the dose cohorts, the y-axis shows the percent change, and the lines represent values from different subjects. FIG. 17C shows the scatter dot plot of percent change in IFN-γ from C1D2 relative to baseline in patients treated with TY21580. The x-axis shows the dose cohorts, the y-axis shows the percent change, and the dots represent values from different subjects.

FIGS. 18A-18B show changes of TNFα in patients treated with TY21580. FIG. 18A shows the change in relative abundance of TNFα to baseline in patients treated with TY21580. The x-axis shows the dose cohorts, the y-axis shows the percent change, and the lines represent values from different subjects. FIG. 18B shows the scatter dot plot of percent change in TNFα from C1D2 relative to baseline treated with TY21580. The x-axis shows the dose cohorts, the y-axis shows the percent change, and the dots represent values from different subjects.

FIGS. 19A and 19B show changes of IL-6 in patients treated with TY21580. FIG. 20A shows the change in relative abundance of IL-6 to baseline in patients treated with TY21580. The x-axis shows the dose cohorts, the y-axis shows the percent change, and the lines represent values from different subjects. FIG. 19B shows the scatter dot plot of percent change in IL-6 from C1D2 relative to baseline in patients treated with TY21580. The x-axis shows the dose cohorts, the y-axis shows the percent change, and the dots represent values from different subjects.

FIGS. 20A and 20B show changes of IL-10 in patients treated with TY21580. FIG. 20A shows the change in relative abundance of IL-10 to baseline in patients treated with TY21580. The x-axis shows the dose cohorts, the y-axis shows the percent change, and the lines represent values from different subjects. FIG. 20B shows the scatter dot plot of percent change in IL-10 from C1D2 relative to baseline in patients treated with TY21580. The x-axis shows the dose cohorts, the y-axis shows the percent change, and the dots represent values from different subjects.

FIG. 21A-21C show changes of sPD-L1 in patients treated with TY21580. FIG. 21A shows the abundance of sPD-L1 in patients treated with TY21580. The x-axis shows the dose cohorts, the y-axis shows the concentration of sPD-L1, and the lines represent values from different subjects. FIG. 21B shows the change in relative abundance of sPD-L1 to baseline in patients treated with TY21580. The x-axis shows the dose cohorts, the y-axis shows the percent change relative to baseline, and the lines represent values from different subjects. FIG. 21C shows the scatter dot plot of percent change in sPD-L1 from C1D8 relative to baseline in patients treated with TY21580. The x-axis shows the dose cohorts, the y-axis shows the percent change, and the dots represent values from different subjects.

FIGS. 22A and 22B show changes of sCD25 in patients treated with TY21580. FIG. 22A shows the change in relative abundance of sCD25 to baseline in patients treated with TY21580. The x-axis shows the dose cohorts, the y-axis shows the percent change, and the lines represent values from different subjects. FIG. 22B shows the scatter dot plot of percent change in sCD25 from C1D8 relative to baseline in patients treated with TY21580. The x-axis shows the dose cohorts, the y-axis shows the percent change, and the dots represent values from different subjects.

FIGS. 23A and 23B show changes of CXCL-11 in patients treated with TY21580. FIG. 23A shows the change in relative abundance of CXCL-11 to baseline in patients treated with TY21580. The x-axis shows the dose cohorts, the y-axis shows the percent change, and the lines represent values from different subjects. FIG. 23B shows the scatter dot plot of percent change in CXCL-11 from C1D8 relative to baseline in patients treated with TY21580. The x-axis shows the dose cohorts, the y-axis shows the percent change, and the dots represent values from different subjects.

FIG. 24A-24E show changes of CD4+ T cells in patients treated with TY21580. FIG. 24A shows the change in CD4+ T cell levels relative to baseline in patients treated with TY21580 over time. The x-axis shows the dose cohorts, the y-axis shows the change in absolute cell level per L relative to baseline, and the lines represent values from different subjects. Values from several patients are highlighted (#4, #19, #22, #23). FIG. 24B shows the percent change in absolute CD4+ T cell levels on C1D8 relative to baseline in patients treated with TY21580. The x-axis shows the dose cohorts, the y-axis shows the percent change, and the dots represent values from different subjects. FIG. 24C shows the change in absolute count of CD4+ T cells from baseline to C1D8 in patients treated with TY21580. The x-axis shows the time point, the y-axis shows the absolute count per μL, and the lines represent values from different subjects. FIG. 24D shows the shows the percent change in absolute CD4+ T cell levels on C1D15 relative to baseline in patients treated with TY21580. The x-axis shows the dose cohorts, the y-axis shows the percent change, and the dots represent values from different subjects. FIG. 24E shows the change in absolute count of CD4+ CD8− T cells from baseline to C1D15 in patients treated with TY21580. The x-axis shows the time point, the y-axis shows the absolute count per μL, and the lines represent values from different subjects.

FIG. 25A-25E show changes of CD8+ T cells in patients treated with TY21580. FIG. 25A shows the change in CD8+ T cell levels relative to baseline in patients treated with TY21580 over time. The x-axis shows the dose cohorts, the y-axis shows the change in absolute cell level per μL relative to baseline, and the lines represent values from different subjects. Values from several patients are highlighted (#4, #19, #22, #23). FIG. 25B shows the percent change in absolute CD8+ T cell levels on C1D8 relative to baseline in patients treated with TY21580. The x-axis shows the dose cohorts, the y-axis shows the percent change, and the dots represent values from different subjects. FIG. 25C shows the change in absolute count of CD8+ T cells from baseline to C1D8 in patients treated with TY21580. The x-axis shows the time point, the y-axis shows the absolute count per μL, and the lines represent values from different subjects. FIG. 25D shows the percent change in absolute CD8+ T cell levels on C1D15 relative to baseline in patients treated with TY21580. The x-axis shows the dose cohorts, the y-axis shows the percent change, and the dots represent values from different subjects. FIG. 25E shows the change in absolute count of CD8+ T cells from baseline to C1D15 in patients treated with TY21580. The x-axis shows the time point, the y-axis shows the absolute count per μL, and the lines represent values from different subjects.

FIGS. 26A and 26B show changes of NK cells in patients treated with TY21580. FIG. 26A shows the change in absolute count of NK cells on C1D8 relative to baseline in patients treated with TY21580. The x-axis shows the time point, the y-axis shows the absolute count per μL, and the lines represent values from different subjects. FIG. 26B shows the change in absolute count of NK cells on C1D15 relative to baseline in patients treated with TY21580. The x-axis shows the time point, the y-axis shows the absolute count per μL, and the lines represent values from different subjects.

FIG. 27 shows a 3D structure of CTLA4 and its interations with Ipilimumab and TY21580.

FIG. 28 shows Treg depletion and CD8/Treg rations in the tumor-infiltrating lymphocytes (TIL) of treated MC38 tumors in hCTLA4 knock-in background.

FIG. 29 shows the plasma Cₘₐₓ of TY21580 at various doses levels following IV administration of TY21580 to human subjects.

FIG. 30 shows the predicted AUC_{(0-∞)} of TY21580 at various doses levels following IV administration of TY21580 to human subjects.

FIG. 31 shows a dimulated PK study used to predict serum concentration levels at various doses of TY21580 administered to human subjects.

### DETAILED DESCRIPTION

### I. Definitions

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular. Generally, nomenclatures used in connection with, and techniques of, antibody engineering, immunotherapy, cell and tissue culture, molecular biology, immunology, microbiology, genetics and protein and nucleic acid chemistry described herein are those well-known and commonly used in the art.

The term “antibody” is used herein in the broadest sense and specifically covers monoclonal antibodies (including full length monoclonal antibodies), polyclonal antibodies, multispecific antibodies (e.g., bispecific antibodies, trispecific antibodies), and antibody fragments (e.g., Fab, Fab′, Fab′-SH, F(ab′)₂, Fv and/or a single-chain variable fragment or scFv) so long as they exhibit the desired biological activity.

In some embodiments, the term “antibody” refers to an antigen-binding protein (i.e., immunoglobulin) having a basic four-polypeptide chain structure consisting of two identical heavy (H) chains and two identical light (L) chains. Each L chain is linked to an H chain by one covalent disulfide bond, while the two H chains are linked to each other by one or more disulfide bonds depending on the H chain isotype. Each heavy chain has, at the N-terminus, a variable region (abbreviated herein as V_{H}) followed by a constant region. The heavy chain constant region is comprised of three domains, C_{H1}, C_{H2} and C_{H3}. Each light chain has, at the N-terminus, a variable region (abbreviated herein as V_{I}) followed by a constant region at its other end. The light chain constant region is comprised of one domain, C_{L}. The V_{L} is aligned with the V_{H} and the C_{L} is aligned with the first constant domain of the heavy chain (CH1). The pairing of a V_{H} and V_{L} together forms a single antigen-binding site. An IgM antibody consists of 5 of the basic heterotetramer units along with an additional polypeptide called J chain, and therefore contains 10 antigen binding sites, while secreted IgA antibodies can polymerize to form polyvalent assemblages comprising 2-5 of the basic 4-chain units along with J chain.

The V_{H} and V_{L} regions can be further subdivided into regions of hypervariability, termed hyper-variable regions (HVR) based on structural and sequence analysis. HVRs are interspersed with regions that are more conserved, termed framework regions (FW) (see e.g., Chen et al. (1999) J. Mol. Biol. (1999) 293, 865-881). Each V_{H} and V_{L} is composed of three HVRs and four FWs, arranged from amino-terminus to carboxy-terminus in the following order: FW-1_HVR-1_FW-2_HVR-2_FW-3_HVR-3_FW4. Throughout the present application, the three HVRs of the heavy chain are referred to as HVR-H1, HVR-H2, and HVR-H3. Similarly, the three HVRs of the light chain are referred to as HVR-L1, HVR-L2, and HVR-L3.

As used herein, the term “CDR” or “complementarity determining region” is intended to mean the non-contiguous antigen combining sites found within the variable region of both heavy and light chain polypeptides. These particular regions have been described by Kabat et al., J. Biol. Chem. 252:6609-6616 (1977); Kabat et al., U.S. Dept. of Health and Human Services, “Sequences of proteins of immunological interest” (1991); Chothia et al., J. Mol. Biol. 196:901-917 (1987); Al-Lazikani B. et al., J. Mol. Biol., 273: 927-948 (1997); MacCallum et al., J. Mol. Biol. 262:732-745 (1996); Abhinandan and Martin, Mol. Immunol., 45: 3832-3839 (2008); Lefranc M. P. et al., Dev. Comp. Immunol., 27: 55-77 (2003); and Honegger and Plückthun, J. Mol. Biol., 309:657-670 (2001), where the definitions include overlapping or subsets of amino acid residues when compared against each other. Nevertheless, application of either definition to refer to a CDR of an antibody or grafted antibodies or variants thereof is intended to be within the scope of the term as defined and used herein. The amino acid residues, which encompass the CDRs as defined by each of the above-cited references, are set forth below in Table I as a comparison. CDR prediction algorithms and interfaces are known in the art, including, for example, Abhinandan and Martin, Mol. Immunol., 45: 3832-3839 (2008); Ehrenmann F. et al., Nucleic Acids Res., 38: D301-D307 (2010); and Adolf-Bryfogle J. et al., Nucleic Acids Res., 43: D432-D438 (2015). The contents of the references cited in this paragraph are incorporated herein by reference in their entireties for use in the present invention and for possible inclusion in one or more claims herein.

TABLE I
CDR DEFINITIONS

| | Kabat¹ | Chothia² | MacCallum³ | IMGT⁴ | AHo⁵ |
|---|---|---|---|---|---|
| VH CDR1 | 31-35 | 26-32 | 30-35 | 27-38 | 25-40 |
| VH CDR2 | 50-65 | 53-55 | 47-58 | 56-65 | 58-77 |
| VH CDR3 | 95-102 | 96-101 | 93-101 | 105-117 | 109-137 |
| VL CDR1 | 24-34 | 26-32 | 30-36 | 27-38 | 25-40 |
| VL CDR2 | 50-56 | 50-52 | 46-55 | 56-65 | 58-77 |
| VL CDR3 | 89-97 | 91-96 | 89-96 | 105-117 | 109-137 |

¹Residue numbering follows the nomenclature of Kabat et al., supra
²Residue numbering follows the nomenclature of Chothia et al., supra
³Residue numbering follows the nomenclature of MacCallum et al., supra
⁴Residue numbering follows the nomenclature of Lefranc et al., supra
⁵Residue numbering follows the nomenclature of Honegger and Plückthun, supra

The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. Within light and heavy chains, the variable and constant regions are joined by a “J” region of about 12 or more amino acids, with the heavy chain also including a “D” region of about 10 or more amino acids (see e.g., Fundamental Immunology Ch. 7 (Paul, W., ed., 2^{nd} ed. Raven Press, N.Y). (1989)).

The L chain from any vertebrate species can be assigned to one of two clearly distinct types, called kappa and lambda, based on the amino acid sequences of their constant domains. Depending on the amino acid sequence of the constant domain of their heavy chains (CH), antibodies can be assigned to different classes or isotypes. There are five classes of antibodies: IgA, IgD, IgE, IgG, and IgM, having heavy chains designated a (alpha), 6 (delta), E (epsilon), γ (gamma), and μ (mu), respectively. The IgG class of antibody can be further classified into four subclasses IgG1, IgG2, IgG3, and IgG4 by the gamma heavy chains, Y1-Y4, respectively.

The term “antibody derivative” or “derivative” of an antibody refers to a molecule that is capable of binding to the same antigen (e.g., CTLA4) that the antibody binds to and comprises an amino acid sequence of the antibody linked to an additional molecular entity. The amino acid sequence of the antibody that is contained in the antibody derivative may be a full-length heavy chain, a full-length light chain, any portion or portions of a full-length heavy chain, any portion or portions of the full-length light chain of the antibody, any other fragment(s) of an antibody, or the complete antibody. The additional molecular entity may be a chemical or biological molecule. Examples of additional molecular entities include chemical groups, amino acids, peptides, proteins (such as enzymes, antibodies), and chemical compounds. The additional molecular entity may have any utility, such as for use as a detection agent, label, marker, pharmaceutical or therapeutic agent. The amino acid sequence of an antibody may be attached or linked to the additional molecular entity by chemical coupling, genetic fusion, noncovalent association, or otherwise. The term “antibody derivative” also encompasses chimeric antibodies, humanized antibodies, and molecules that are derived from modifications of the amino acid sequences of a CTLA4 antibody, such as conservation amino acid substitutions, additions, and insertions.

The term “antigen-binding fragment” or “antigen binding portion” of an antibody refers to one or more portions of an antibody that retain the ability to bind to the antigen that the antibody bonds to (e.g., CTLA4). Examples of “antigen-binding fragments” of an antibody include (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an antibody, (v) a dAb fragment (Ward et al., Nature 341:544-546 (1989)), which consists of a V_{H} domain; and (vi) an isolated complementarity determining region (CDR).

The term “CTLA4” is used in the present application, and includes the human CTLA4 (e.g., UniProt accession number P16410), as well as variants, isoforms, and species homologs thereof (e.g., mouse CTLA4 (UniProt accession number P09793), rat CTLA4 (UniProt accession number Q9Z1A7), dog CTLA4 (UniProt accession number Q9XSI1), cynomolgus monkey CTLA4 (UniProt accession number G7PL88), etc.). Accordingly, an anti-CTLA4 antibody, as defined and disclosed herein, may also bind CTLA4 from species other than human. In other cases, an anti-CTLA4 antibody may be completely specific for the human CTLA4 and may not exhibit species or other types of cross-reactivity.

The term “CTLA4 antibody” refers to an antibody, as defined herein, capable of binding to human CTLA4.

The term “chimeric antibody” refers to an antibody that comprises amino acid sequences derived from different animal species, such as those having a variable region derived from a human antibody and a murine immunoglobulin constant region.

The term “compete for binding” refers to the interaction of two antibodies in their binding to a binding target. A first antibody competes for binding with a second antibody if binding of the first antibody with its cognate epitope is detectably decreased in the presence of the second antibody compared to the binding of the first antibody in the absence of the second antibody. The alternative, where the binding of the second antibody to its epitope is also detectably decreased in the presence of the first antibody, can, but need not, be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope, whether to the same, greater, or lesser extent, the antibodies are said to “cross-compete” with each other for binding of their respective epitope(s).

The term “epitope” refers to a part of an antigen to which an antibody (or antigen-binding fragment thereof) binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope can include various numbers of amino acids in a unique spatial conformation. Methods of determining spatial conformation of epitopes include, for example, x-ray crystallography, 2-dimensional nuclear magnetic resonance, deuterium and hydrogen exchange in combination with mass spectrometry, or site-directed mutagenesis, or all methods used in combination with computational modeling of antigen and its complex structure with its binding antibody and its variants (see e.g., Epitope Mapping Protocols in Methods in Molecular Biology, Vol. 66, G. E. Morris, Ed. (1996)). Once a desired epitope of an antigen is determined, antibodies to that epitope can be generated, e.g., using the techniques described herein. The generation and characterization of antibodies may also elucidate information about desirable epitopes. From this information, it is then possible to competitively screen antibodies for binding to the same epitope. An approach to achieve this is to conduct cross-competition studies to find antibodies that competitively bind with one another, i.e., the antibodies compete for binding to the antigen. A high throughput process for “binning” antibodies based upon their cross-competition is described in PCT Publication No. WO 03/48731.

The term “glycosylation sites” refers to amino acid residues which are recognized by a eukaryotic cell as locations for the attachment of sugar residues. The amino acids where carbohydrate, such as oligosaccharide, is attached are typically asparagine (N-linkage), serine (0-linkage), and threonine (0-linkage) residues. The specific site of attachment is typically signaled by a sequence of amino acids, referred to herein as a “glycosylation site sequence”. The glycosylation site sequence for N-linked glycosylation is: -Asn-X-Ser- or -Asn-X-Thr-, where X may be any of the conventional amino acids, other than proline. The terms “N-linked” and “O-linked” refer to the chemical group that serves as the attachment site between the sugar molecule and the amino acid residue. N-linked sugars are attached through an amino group; O-linked sugars are attached through a hydroxyl group. The term “glycan occupancy” refers to the existence of a carbohydrate moiety linked to a glycosylation site (i.e., the glycan site is occupied). Where there are at least two potential glycosylation sites on a polypeptide, either none (0-glycan site occupancy), one (1-glycan site occupancy) or both (2-glycan site occupancy) sites can be occupied by a carbohydrate moiety.

The term “host cell” refers to a cellular system, which can be engineered to generate proteins, protein fragments, or peptides of interest. Host cells include, without limitation, cultured cells, e.g., mammalian cultured cells derived from rodents (rats, mice, guinea pigs, or hamsters) such as CHO, BHK, NSO, SP2/0, YB2/0; human cells (e.g., HEK293F cells, HEK293T cells; or human tissues or hybridoma cells, yeast cells, insect cells (e.g., S2 cells), bacterial cells (e.g., E. coli cells) and cells comprised within a transgenic animal or cultured tissue. The term encompasses not only the particular subject cell but also the progeny of such a cell. Because certain modifications may occur in succeeding generations due to either mutation or environmental influences, such progeny may not be identical to the parent cell, but are still included within the scope of the term “host cell.”

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

The term “humanized antibody” refers to a chimeric antibody that contains amino acid residues derived from human antibody sequences. A humanized antibody may contain some or all of the CDRs or HVRs from a non-human animal or synthetic antibody while the framework and constant regions of the antibody contain amino acid residues derived from human antibody sequences.

The term “illustrative antibody” refers to any one of the antibodies described in the disclosure and designated as those listed in Tables A and B, and any antibodies comprising the 6 HVRs and/or the VH and VLs of the antibodies listed in Tables A and B. These antibodies may be in any class (e.g., IgA, IgD, IgE, IgG, and IgM). Thus, each antibody identified above encompasses antibodies in all five classes that have the same amino acid sequences for the VL and VH regions. Further, the antibodies in the IgG class may be in any subclass (e.g., IgG1 IgG2, IgG3, and IgG4). Thus, each antibody identified above in the IgG subclass encompasses antibodies in all four subclasses that have the same amino acid sequences for the VL and V_{H} regions. The amino acid sequences of the heavy chain constant regions of human antibodies in the five classes, as well as in the four IgG subclasses, are known in the art.

“Antibody-dependent cell-mediated cytotoxicity” or “ADCC” refers to a form of cytotoxicity in which secreted immunoglobulin bound onto Fc receptors (FcRs) present on certain cytotoxic cells (e.g. NK cells, neutrophils, and macrophages) enable these cytotoxic effector cells to bind specifically to an antigen-bearing target cell and subsequently kill the target cell with cytotoxins. The primary cells for mediating ADCC, NK cells, express FcγRIII only, whereas monocytes express FcγRI, FcγRII, and FcγRIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 or U.S. Pat. No. 6,737,056 (Presta), may be performed. Useful effector cells for such assays include PBMC and NK cells. Alternatively, or additionally, ADCC activity of the molecule of interest may be assessed in vivo, e.g., in an animal model such as that disclosed in Clynes et al. PNAS (USA) 95:652-656 (1998). An exemplary assay for assessing ADCC activity is provided in the examples herein.

“Complement dependent cytotoxicity” or “CDC” refers to the lysis of a target cell in the presence of complement. Activation of the classical complement pathway is initiated by the binding of the first component of the complement system (Clq) to antibodies (of the appropriate subclass), which are bound to their cognate antigen. To assess complement activation, a CDC assay, e.g., as described in Gazzano-Santoro et al., J. Immunol. Methods 202:163 (1996), may be performed. Polypeptide variants with altered Fc region amino acid sequences (polypeptides with a variant Fc region) and increased or decreased Clq binding capability are described, e.g., in U.S. Pat. No. 6,194,551 BI and WO 1999/51642. See also, e.g., Idusogie et al. J. Immunol. 164: 4178-4184 (2000).

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see e.g., Flatman et al., J. Chromatogr. B 848:79-87 (2007).

The term “Kₐ” refers to the association rate constant of a particular antibody-antigen interaction, where the term “k_{d}” refers to the dissociation rate constant of a particular antibody-antigen interaction.

The term “K_{D}” refers to the equilibrium dissociation constant of a particular antibody-antigen interaction. It is obtained from the ratio of k_{d} to kₐ (i.e., k_{d}/kₐ) and is expressed as a molar concentration (M). K_{D} is used as a measure for the affinity of an antibody's binding to its binding partner. The smaller the K_{D}, the more tightly bound the antibody is, or the higher the affinity between antibody and the antigen. For example, an antibody with a nanomolar (nM) dissociation constant binds more tightly to a particular antigen than an antibody with a micromolar (μM) dissociation constant. K_{D} values for antibodies can be determined using methods well established in the art. One method for determining the K_{D} of an antibody is by using surface plasmon resonance, typically using a biosensor system such as a BIACORE® system. For example, an assay procedure using the BIACORE™ system (BIAcore assay) is described in at least Example 3 of the present application.

The term “mammal” refers to any animal species of the Mammalia class. Examples of mammals include: humans; laboratory animals such as rats, mice, hamsters, rabbits, non-human primates, and guinea pigs; domestic animals such as cats, dogs, cattle, sheep, goats, horses, and pigs; and captive wild animals such as lions, tigers, elephants, and the like.

As used herein, “sequence identity” between two polypeptide sequences indicates the percentage of amino acids that are identical between the sequences. The amino acid sequence identity of polypeptides can be determined conventionally using known computer programs such as Bestfit, FASTA, or BLAST (see e.g., Pearson, Methods Enzymol. 183:63-98 (1990); Pearson, Methods Mol. Biol. 132:185-219 (2000); Altschul et al., J. Mol. Biol. 215:403-410 (1990); Altschul et al., Nucleic Acids Res. 25:3389-3402 (1997)). When using Bestfit or any other sequence alignment program to determine whether a particular sequence is, for instance, 95% identical to a reference amino acid sequence, the parameters are set such that the percentage of identity is calculated over the full length of the reference amino acid sequence and that gaps in homology of up to 5% of the total number of amino acid residues in the reference sequence are allowed. This aforementioned method in determining the percentage of identity between polypeptides is applicable to all proteins, fragments, or variants thereof disclosed herein.

As used herein, the term “binds”, “binds to”, “specifically binds” “specifically binds to” or is “specific for” refers to measurable and reproducible interactions such as binding between a target and an antibody, which is determinative of the presence of the target in the presence of a heterogeneous population of molecules including biological molecules. For example, an antibody that binds to or specifically binds to a target (which can be an epitope) is an antibody that binds this target with greater affinity, avidity, more readily, and/or with greater duration than it binds to other targets. In one embodiment, the extent of binding of an antibody to an unrelated target is less than about 10% of the binding of the antibody to the target as measured, e.g., by a radioimmunoassay (RIA). In certain embodiments, an antibody that specifically binds to a target has a dissociation constant (Kd) of ≤1 M, ≤100 nM, ≤10 nM, ≤1 nM, or ≤0.1 nM. In certain embodiments, an antibody specifically binds to an epitope on a protein that is conserved among the protein from different species. In another embodiment, specific binding can include, but does not require exclusive binding.

The term “treat”, “treating”, or “treatment”, with reference to a certain disease condition in a mammal, refers causing a desirable or beneficial effect in the mammal having the disease condition. The desirable or beneficial effect may include reduced frequency or severity of one or more symptoms of the disease (i.e., tumor growth and/or metastasis, or other effect mediated by the numbers and/or activity of immune cells, and the like), or arrest or inhibition of further development of the disease, condition, or disorder. In the context of treating cancer in a mammal, the desirable or beneficial effect may include inhibition of further growth or spread of cancer cells, death of cancer cells, inhibition of reoccurrence of cancer, reduction of pain associated with the cancer, or improved survival of the mammal. The effect can be either subjective or objective. For example, if the mammal is human, the human may note improved vigor or vitality or decreased pain as subjective symptoms of improvement or response to therapy. Alternatively, the clinician may notice a decrease in tumor size or tumor burden based on physical exam, laboratory parameters, tumor markers or radiographic findings. Some laboratory signs that the clinician may observe for response to treatment include normalization of tests, such as white blood cell count, red blood cell count, platelet count, erythrocyte sedimentation rate, and various enzyme levels. Additionally, the clinician may observe a decrease in a detectable tumor marker. Alternatively, other tests can be used to evaluate objective improvement, such as sonograms, nuclear magnetic resonance testing and positron emissions testing.

The term “prevent” or “preventing,” with reference to a certain disease condition in a mammal, refers to preventing or delaying the onset of the disease, or preventing the manifestation of clinical or subclinical symptoms thereof.

The term “isolated nucleic acid” refers to a nucleic acid molecule of genomic, cDNA, or synthetic origin, or a combination thereof, which is separated from other nucleic acid molecules present in the natural source of the nucleic acid. For example, with regard to genomic DNA, the term “isolated” includes nucleic acid molecules which are separated from the chromosome with which the genomic DNA is naturally associated. Preferably, an “isolated” nucleic acid is free of sequences which naturally flank the nucleic acid (i.e., sequences located at the 5′ and 3′ ends of the nucleic acid of interest.

The term “vector” refers to a nucleic acid molecule capable of transporting a foreign nucleic acid molecule. The foreign nucleic acid molecule is linked to the vector nucleic acid molecule by a recombinant technique, such as ligation or recombination. This allows the foreign nucleic acid molecule to be multiplied, selected, further manipulated or expressed in a host cell or organism. A vector can be a plasmid, phage, transposon, cosmid, chromosome, virus, or virion. One type of vectors can be integrated into the genome of a host cell upon introduction into the host cell, and thereby are replicated along with the host genome (e.g., non-episomal mammalian vectors). Another type of vector is capable of autonomous replication in a host cell into which it is introduced (e.g., bacterial vectors having a bacterial origin of replication and episomal mammalian vectors). Another specific type of vector capable of directing the expression of expressible foreign nucleic acids to which they are operatively linked is commonly referred to as “expression vectors.” Expression vectors generally have control sequences that drive expression of the expressible foreign nucleic acids. Simpler vectors, known as “transcription vectors,” are only capable of being transcribed but not translated: they can be replicated in a target cell but not expressed. The term “vector” encompasses all types of vectors regardless of their function. Vectors capable of directing the expression of expressible nucleic acids to which they are operatively linked are commonly referred to “expression vectors.” Other examples of “vectors” may include display vectors (e.g., vectors that direct expression and display of an encoded polypeptide on the surface of a virus or cell (such as a bacterial cell, yeast cell, insect cell, and/or mammalian cell).

As used herein, a “subject”, “patient”, or “individual” may refer to a human or a non-human animal. A “non-human animal” may refer to any animal not classified as a human, such as domestic, farm, or zoo animals, sports, pet animals (such as dogs, horses, cats, cows, etc.), as well as animals used in research. Research animals may refer without limitation to nematodes, arthropods, vertebrates, mammals, frogs, rodents (e.g., mice or rats), fish (e.g., zebrafish or pufferfish), birds (e.g., chickens), dogs, cats, and non-human primates (e.g., rhesus monkeys, cynomolgus monkeys, chimpanzees, etc.). In some embodiments, the subject, patient, or individual is a human.

An “effective amount” refers to at least an amount effective, at dosages and for periods of time necessary, to achieve one or more desired or indicated effects, including a therapeutic or prophylactic result. An effective amount can be provided in one or more administrations. For purposes of the present application, an effective amount of antibody, drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition (e.g., an effective amount as administered as a monotherapy or combination therapy). Thus, an “effective amount” may be considered in the context of administering one or more therapeutic agents, and a single agent may be considered to be given in an effective amount if, in conjunction with one or more other agents, a desirable result may be or is achieved.

The terms “recurrence,” “relapse” or “relapsed” refers to the return of a cancer or disease after clinical assessment of the disappearance of disease. A diagnosis of distant metastasis or local recurrence can be considered a relapse.

The term “refractory” or “resistant” refers to a cancer or disease that has not responded to treatment.

As used herein, “complete response” or “CR” refers to disappearance of all target lesions; “partial response” or “PR” refers to at least a 30% decrease in the sum of the longest diameters (SLD) of target lesions, taking as reference the baseline SLD; and “stable disease” or “SD” refers to neither sufficient shrinkage of target lesions to qualify for PR, nor sufficient increase to qualify for PD, taking as reference the smallest SLD since the treatment started.

As used herein, “progressive disease” or “PD” refers to at least a 20% increase in the SLD of target lesions, taking as reference the smallest SLD recorded since the treatment started or the presence of one or more new lesions.

As used herein, “progression free survival” (PFS) refers to the length of time during and after treatment during which the disease being treated (e.g., cancer) does not get worse. Progression-free survival may include the amount of time patients have experienced a complete response or a partial response, as well as the amount of time patients have experienced stable disease.

As used herein, “overall response rate” (ORR) refers to the sum of complete response (CR) rate and partial response (PR) rate.

As used herein, “overall survival” refers to the percentage of individuals in a group who are likely to be alive after a particular duration of time.

As used herein, the term “biomarker” or “marker” refers generally to a molecule (e.g., pre-mRNA, mRNA, protein, etc.) or cell population (e.g., effector memory T cell or T_{reg} cell, or regulatory T cell or T_{reg} cell), the level of which in or on a subject's tissue (e.g., tumor), or in case of a molecule, secreted by the subject's tissue or cell, can be detected by known methods (or methods disclosed herein) and is predictive or can be used to predict (or aid prediction) for a subject's sensitivity to, and in some embodiments, to predict (or aid prediction) a subject's responsiveness to, treatment regimens (e.g., treatments with an anti-CTLA4 antibody).

As used herein, the term “sample”, refers to a composition that is obtained or derived from a subject of interest that contains a cellular and/or other molecular entity that is to be characterized and/or identified, for example based on physical, biochemical, chemical and/or physiological characteristics.

As used herein, the term “tissue or cell sample” refers to a collection of similar cells obtained from a tissue of a subject or patient. The source of the tissue or cell sample may be solid tissue as from a fresh, frozen and/or preserved organ or tissue sample or biopsy or aspirate; blood or any blood constituents; bodily fluids such as cerebral spinal fluid, amniotic fluid, peritoneal fluid, or interstitial fluid; cells from any time in gestation or development of the subject. The tissue sample may also be primary or cultured cells. Optionally, the tissue or cell sample is obtained from a disease tissue or organ. The tissue sample may contain compounds, which are not naturally intermixed with the tissue in nature such as preservatives, anticoagulants, buffers, fixatives, nutrients, antibiotics, or the like. As used herein, a “reference value” or “reference level” may be an absolute value; a relative value; a value that has an upper and/or lower limit; a range of values; an average value; a median value; a mean value; or a value as compared to a particular level or baseline level.

As used herein, a “baseline level” or “baseline value” refers to a level or a value of a subject before the subject begins a treatment, such as an anti-CTLA4 antibody treatment.

A “reference sample”, “reference cell”, “reference tissue”, “control sample”, “control cell”, or “control tissue”, as used herein, refers to a sample, cell, tissue, standard, or level that is used for comparison purposes. In one embodiment, a reference sample, reference cell, reference tissue, control sample, control cell, or control tissue is obtained from a healthy and/or non-diseased part of the body (e.g., tissue or cells) of the same subject or individual. For example, healthy and/or non-diseased cells or tissue adjacent to the diseased cells or tissue (e.g., cells or tissue adjacent to a tumor). In another embodiment, a reference sample is obtained from an untreated tissue and/or cell of the body of the same subject or individual. In yet another embodiment, a reference sample, reference cell, reference tissue, control sample, control cell, or control tissue is obtained from a healthy and/or non-diseased part of the body (e.g., tissues or cells) of an individual who is not the subject or individual. In even another embodiment, a reference sample, reference cell, reference tissue, control sample, control cell, or control tissue is obtained from an untreated tissue and/or cell of the body of an individual who is not the subject or individual.

By “correlate” or “correlating” is meant comparing, in any way, the performance and/or results of a first analysis or protocol with the performance and/or results of a second analysis or protocol. For example, one may use the results of a first analysis or protocol in carrying out a second protocols and/or one may use the results of a first analysis or protocol to determine whether a second analysis or protocol should be performed. With respect to the embodiment of biomarker analysis or protocol, one may use the results of the biomarker level analysis or protocol to determine whether a specific therapeutic regimen should be performed. With respect to the embodiment of biomarker level analysis or protocol, one may use the results of the biomarker level analysis or protocol to determine whether a specific therapeutic regimen should be performed.

An “effective response” of a patient or a patient's “responsiveness” to treatment with a medicament and similar wording refers to the clinical or therapeutic benefit imparted to a patient at risk for, or suffering from, a disease or disorder, such as cancer. In one embodiment, such benefit includes any one or more of: extending survival (including overall survival and progression free survival); resulting in an objective response (including a complete response or a partial response); or improving signs or symptoms of cancer.

A patient who “does not have an effective response” to treatment refers to a patient who does not have any one of extending survival (including overall survival and progression free survival); resulting in an objective response (including a complete response or a partial response); or improving signs or symptoms of cancer.

The methods and techniques of the present application are generally performed according to methods well known in the art and as described in various general and more specific references that are cited and discussed throughout the present specification unless otherwise indicated. Such references include, e.g., Sambrook and Russell, Molecular Cloning, A Laboratory Approach, Cold Spring Harbor Press, Cold Spring Harbor, N.Y. (2001), Ausubel et al., Current Protocols in Molecular Biology, John Wiley & Sons, NY (2002), and Harlow and Lane Antibodies: A Laboratory Manual, Cold Spring Harbor Laboratory Press, Cold Spring Harbor, N.Y. (1990). Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

As used herein, the twenty conventional amino acids and their abbreviations follow conventional usage. See Immunology—A Synthesis (2nd Edition, E. S. Golub and D. R. Gren, Eds., Sinauer Associates, Sunderland, Mass. (1991)).

As used herein, the singular forms “a”, “an” and “the” include plural referents unless the content clearly dictates otherwise. Thus, for example, reference to “a molecule” optionally includes a combination of two or more such molecules, and the like.

The term “about” as used herein refers to the usual error range for the respective value readily known to the skilled person in this technical field. Reference to “about” a value or parameter herein includes (and describes) embodiments that are directed to that value or parameter per se.

It is understood that aspects and embodiments of the present application described herein include “comprising,” “consisting,” and “consisting essentially of” aspects and embodiments.

As used herein, reference to “not” a value or parameter generally means and describes “other than” a value or parameter. For example, the method is not used to treat cancer of type X means the method is used to treat cancer of types other than X.

The term “about X-Y” used herein has the same meaning as “about X to about Y.”

The term “and/or” as used herein a phrase such as “A and/or B” is intended to include both A and B; A or B; A (alone); and B (alone). Likewise, the term “and/or” as used herein a phrase such as “A, B, and/or C” is intended to encompass each of the following embodiments: A, B, and C; A, B, or C; A or C; A or B; B or C; A and C; A and B; B and C; A (alone); B (alone); and C (alone).

### II. Methods of Treatment

The present application provides methods for treating cancers in a subject using an anti-CTLA4 antibody that specifically binds to human CTLA4. Any one of the anti-CTLA4 antibodies (including full-length antibodies and antigen-binding fragments thereof) in Section IV “Anti-CTLA4 Antibodies” may be used in the methods described herein.

In some embodiments, there is provided a method of treating a cancer in a subject, comprising administering to the subject an effective amount of an anti-CTLA4 antibody, wherein the antibody specifically binds to an epitope comprising amino acid residues Y105 and L106 of human CTLA4 but does not comprise residue I108, wherein the numbering of the amino acid residues is according to SEQ ID NO: 108, and wherein the cancer is resistant or refractory to a prior therapy, wherein the prior therapy is an inhibitor of CTLA4, PD-1, or a PD-1 ligand. In some embodiments, the cancer is a solid cancer, such as advanced-stage and/or metastatic cancer. In some embodiments, the cancer is urothelial carcinoma.

In some embodiments, there is provided a method of treating a cancer in a subject, comprising administering to the subject an effective amount of an anti-CTLA4 antibody, wherein the antibody specifically binds to an epitope comprising amino acid residues Y105 and L106 of human CTLA4 but does not comprise residue I108, wherein the numbering of the amino acid residues is according to SEQ ID NO: 108, and wherein the cancer is resistant or refractory to a different anti-CTLA4 antibody, such as ipilimumab. In some embodiments, the cancer is a solid cancer, such as advanced-stage and/or metastatic cancer. In some embodiments, the cancer is urothelial carcinoma.

In some embodiments, there is provided a method of treating a cancer in a subject, comprising administering to the subject an effective amount of an anti-CTLA4 antibody, wherein the antibody specifically binds to an epitope comprising amino acid residues Y105 and L106 of human CTLA4 but does not comprise residue I108, wherein the numbering of the amino acid residues is according to SEQ ID NO: 108, and wherein the cancer is resistant or refractory to an inhibitor of PD-1 or a PD-1 ligand (e.g., PD-L1 or PD-L2). In some embodiments, the cancer is a solid cancer, such as advanced-stage and/or metastatic cancer. In some embodiments, the cancer is urothelial carcinoma.

In some embodiments, there is provided a method of treating a cancer in a subject, comprising administering to the subject an effective amount of an anti-CTLA4 antibody, wherein the antibody specifically binds to an epitope comprising amino acid residues Y105 and L106 of human CTLA4 but does not comprise residue I108, wherein the numbering of the amino acid residues is according to SEQ ID NO: 108, and wherein the cancer is resistant or refractory to an anti-PD-1 antibody, such as pembrolizumab. In some embodiments, the cancer is a solid cancer, such as advanced-stage and/or metastatic cancer. In some embodiments, the cancer is urothelial carcinoma.

In some embodiments, there is provided a method of treating a cancer in a subject, wherein the cancer is resistant or refractory to an inhibitor of CTLA-4, PD-1 or a PD-1 ligand (e.g., PD-L1 or PD-L2), comprising administering to the subject an effective amount of an anti-CTLA4 antibody, wherein the antibody comprises: (a) a heavy chain variable region comprising an HVR-H1 comprising the amino acid sequence of SEQ ID NO: 23, an HVR-H2 comprising the amino acid sequence of SEQ ID NO: 35, and an HVR-H3 comprising the amino acid sequence of SEQ ID NO: 45, and/or a light chain variable region comprising an HVR-L1 comprising the amino acid sequence of SEQ ID NO: 58, an HVR-L2 comprising the amino acid sequence of SEQ ID NO: 66, and an HVR-L3 comprising the amino acid sequence of SEQ ID NO: 75. In some embodiments, the cancer is resistant or refractory to an anti-PD-1 antibody, such as pembrolizumab. In some embodiments, the cancer is resistant or refractory to a different anti-CTLA4 antibody, such as ipilimumab. In some embodiments, the cancer is resistant or refractory to an anti-PD-L1 antibody. In some embodiments, the cancer is a solid cancer, such as advanced-stage and/or metastatic cancer. In some embodiments, the cancer is urothelial carcinoma. In some embodiments, the antibody comprises a heavy chain variable region comprising the amino acid sequence of SEQ ID NO: 87 or an amino acid sequence having at least 90% sequence identity to the amino acid sequence of SEQ ID NO: 87, and/or a light chain variable region comprising the amino acid sequence of SEQ ID NO: 100 or an amino acid sequence having at least 90% sequence identity to the amino acid sequence of SEQ ID NO: 100. In some embodiments, the antibody comprises a human IgG1 Fc region, such as a wildtype IgG1 Fc region or a variant that has enhanced ADCC activity. In some embodiments, the antibody is TY21580.

In some embodiments, there is provided a method of treating a cancer in a subject, comprising administering to the subject an effective amount of an anti-CTLA4 antibody, wherein the antibody specifically binds to an epitope comprising amino acid residues Y105 and L106 of human CTLA4 but does not comprise residue I108, wherein the numbering of the amino acid residues is according to SEQ ID NO: 108, and wherein the anti-CTLA4 antibody is administered at a dose of at least about 6 mg/kg (e.g., 6 mg/kg or 10 mg/kg). In some embodiments, the anti-CTLA4 antibody comprises: (a) a heavy chain variable region comprising an HVR-H1 comprising the amino acid sequence of SEQ ID NO: 23, an HVR-H2 comprising the amino acid sequence of SEQ ID NO: 35, and an HVR-H3 comprising the amino acid sequence of SEQ ID NO: 45, and/or a light chain variable region comprising an HVR-L1 comprising the amino acid sequence of SEQ ID NO: 58, an HVR-L2 comprising the amino acid sequence of SEQ ID NO: 66, and an HVR-L3 comprising the amino acid sequence of SEQ ID NO: 75. In some embodiments, the antibody comprises a heavy chain variable region comprising the amino acid sequence of SEQ ID NO: 87 or an amino acid sequence having at least 90% sequence identity to the amino acid sequence of SEQ ID NO: 87, and/or a light chain variable region comprising the amino acid sequence of SEQ ID NO: 100 or an amino acid sequence having at least 90% sequence identity to the amino acid sequence of SEQ ID NO: 100. In some embodiments, the antibody comprises a human IgG1 Fc region, such as a wildtype IgG1 Fc region or a variant that has enhanced ADCC activity. In some embodiments, the antibody is TY21580. In some embodiments, the anti-CTLA4 antibody is administered about once every three weeks. In some embodiments, the anti-CTLA4 antibody is administered intravenously.

In some embodiments, there is provided a method of treating a cancer in a subject, comprising administering to the subject: (a) an effective amount of an anti-CTLA4 antibody, wherein the antibody specifically binds to an epitope comprising amino acid residues Y105 and L106 of human CTLA4 but does not comprise residue I108, wherein the numbering of the amino acid residues is according to SEQ ID NO: 108, and (b) an effective amount of an anti-PD-1 antibody. In some embodiments, the anti-PD-1 antibody is pembrolizumab, a biosimilar thereof, or a derivative thereof. In some embodiments, the anti-PD-1 antibody is toripalimab, a biosimilar thereof, or a derivative thereof. In some embodiments, the anti-CTLA4 antibody comprises: (a) a heavy chain variable region comprising an HVR-H1 comprising the amino acid sequence of SEQ ID NO: 23, an HVR-H2 comprising the amino acid sequence of SEQ ID NO: 35, and an HVR-H3 comprising the amino acid sequence of SEQ ID NO: 45, and/or a light chain variable region comprising an HVR-L1 comprising the amino acid sequence of SEQ ID NO: 58, an HVR-L2 comprising the amino acid sequence of SEQ ID NO: 66, and an HVR-L3 comprising the amino acid sequence of SEQ ID NO: 75. In some embodiments, the antibody comprises a heavy chain variable region comprising the amino acid sequence of SEQ ID NO: 87 or an amino acid sequence having at least 90% sequence identity to the amino acid sequence of SEQ ID NO: 87, and/or a light chain variable region comprising the amino acid sequence of SEQ ID NO: 100 or an amino acid sequence having at least 90% sequence identity to the amino acid sequence of SEQ ID NO: 100. In some embodiments, the antibody comprises a human IgG1 Fc region, such as a wildtype IgG1 Fc region or a variant that has enhanced ADCC activity. In some embodiments, the antibody is TY21580. In some embodiments, the anti-CTLA4 antibody is administered at a dose of at least about 6 mg/kg (e.g., 6 mg/kg or 10 mg/kg). In some embodiments, the anti-CTLA4 antibody is administered about once every three weeks. In some embodiments, the anti-CTLA4 antibody is administered intravenously. In some embodiments, the cancer is resistant or refractory to an inhibitor of CTLA-4, PD-1 or a PD-1 ligand (e.g., PD-L1 or PD-L2). In some embodiments, the cancer is a solid cancer, such as advanced-stage and/or metastatic cancer. In some embodiments, the cancer is urothelial carcinoma. Cancer treatments can be evaluated by, e.g., tumor regression, tumor weight or size shrinkage, time to progression, duration of survival, progression free survival, overall response rate, duration of response, quality of life, protein expression and/or activity. Approaches to determining efficacy of therapy can be employed, including for example, measurement of response through radiological imaging.

The anti-CTLA4 antibodies and compositions provided by the present disclosure can be administered via any suitable enteral route or parenteral route of administration. The term “enteral route” of administration refers to the administration via any part of the gastrointestinal tract. Examples of enteral routes include oral, mucosal, buccal, and rectal route, or intragastric route. “Parenteral route” of administration refers to a route of administration other than enteral route. Examples of parenteral routes of administration include intravenous, intramuscular, intradermal, intraperitoneal, intratumor, intravesical, intraarterial, intrathecal, intracapsular, intraorbital, intracardiac, transtracheal, intraarticular, subcapsular, subarachnoid, intraspinal, epidural and intrasternal, subcutaneous, or topical administration. The antibodies and compositions of the disclosure can be administered using any suitable method, such as by oral ingestion, nasogastric tube, gastrostomy tube, injection, infusion, implantable infusion pump, and osmotic pump. The suitable route and method of administration may vary depending on a number of factors such as the specific antibody being used, the rate of absorption desired, specific formulation or dosage form used, type or severity of the disorder being treated, the specific site of action, and conditions of the patient, and can be readily selected by a person skilled in the art. In some embodiments, the anti-CTLA4 antibody is administered intravenously.

In some embodiments, the anti-CTLA4 antibody is administered at a dose of no more than any one of 20 mg/kg, 15 mg/kg, 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.8 mg/kg, 0.6 mg/kg, 0.5 mg/kg, 0.4 mg/kg, 0.3 mg/kg, 0.2 mg/kg, 0.1 mg/kg, 0.08 mg/kg, 0.05 mg/kg, 0.04 mg/kg, 0.03 mg/kg, 0.01 mg/kg, 0.003 mg/kg, or 0.001 mg/kg. In some embodiments, the dose of the anti-CTLA4 antibody is within any one of the following ranges, wherein the ranges have an upper limit of any one of: 20 mg/kg, 15 mg/kg, 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.8 mg/kg, 0.6 mg/kg, 0.5 mg/kg, 0.4 mg/kg, 0.3 mg/kg, 0.2 mg/kg, 0.1 mg/kg, 0.08 mg/kg, 0.05 mg/kg, 0.04 mg/kg, 0.03 mg/kg, or 0.003 mg/kg, and an independently selected lower limit of any one of 15 mg/kg, 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.8 mg/kg, 0.6 mg/kg, 0.5 mg/kg, 0.4 mg/kg, 0.3 mg/kg, 0.2 mg/kg, 0.1 mg/kg, 0.08 mg/kg, 0.05 mg/kg, 0.04 mg/kg, 0.03 mg/kg, 0.01 mg/kg, 0.003 mg/kg or 0.001 mg/kg, and wherein the lower limit is less than the upper limit. In some embodiments, the anti-CTLA4 antibody is administered at a dose of any one of about 0.03 mg/kg to about 20 mg/kg, about 0.1 mg/kg to about 20 mg/kg, about 0.3 mg/kg to about 20 mg/kg, about 1 mg/kg to about 20 mg/kg, about 5 mg/kg to about 20 mg/kg, about 0.03 mg/kg to about 10 mg/kg, about 0.1 mg/kg to about 10 mg/kg, about 0.3 mg/kg to about 10 mg/kg, about 1 mg/kg to about 10 mg/kg, about 3 mg/kg to about 10 mg/kg, about 5 mg/kg to about 10 mg/kg, about 0.03 mg/kg to about 0.1 mg/kg, about 0.1 mg/kg to about 0.3 mg/kg, about 0.3 mg/kg to about 1 mg/kg, about 1 mg/kg to about 3 mg/kg, about 3 mg/kg to about 5 mg/kg, about 0.1 mg/kg to about 3 mg/kg, or about 1 mg/kg to about 5 mg/kg. The doses described herein may refer to a suitable dose for a human, or an equivalent dose for the specific species of the subject. In some embodiments, the anti-CTLA4 antibody is administered at a dose equivalent to about 0.03 mg/kg to about 10 mg/kg, or about 0.03 mg/kg to about 20 mg/kg. such as about 0.03 mg/kg, about 0.1 mg/kg, about 0.3 mg/kg, about 1.0 mg/kg, about 3.0 mg/kg, about 6.0 mg/kg, about 10 mg/kg, about 15 mg/kg, or about 20 mg/kg for a human subject.

In some embodiments where the anti-CTLA4 antibody comprises a heavy chain variable region comprising the amino acid sequence of SEQ ID NO: 87 or an amino acid sequence having at least 90% sequence identity to the amino acid sequence of SEQ ID NO: 87, and/or a light chain variable region comprising the amino acid sequence of SEQ ID NO: 100 or an amino acid sequence having at least 90%/o sequence identity to the amino acid sequence of SEQ ID NO: 100, the anti-CTLA4 antibody is administered at a dose of between about 0.5 mg/kg to about 10 mg/kg once every three weeks, about 1 mg/kg to about 10 mg/kg once every three weeks, about 3 mg/kg to about 20 mg/kg once every three weeks, about 3 mg/kg to about 15 mg/kg once every three weeks, about 6 mg/kg to about 15 mg/kg once every three weeks, or from abut 6 mg/kg to about 10 mg/kg once every three weeks. In some such embodiments, the anti-CTLA4 antibody is administered at a dose of about 0.5 mg/kg once every three weeks. In some such embodiments, the anti-CTLA4 antibody is administered at a dose of about 1 mg/kg once every three weeks. In some such embodiments, the anti-CTLA4 antibody is administered at a dose of about 3 mg/kg once every three weeks. In other such embodiments, the anti-CTLA4 antibody is administered at a dose of about 6 mg/kg once every three weeks. In other such embodiments, the anti-CTLA4 antibody is administered at a dose of about 10 mg/kg once every three weeks. In some of the foregoing embodiments, the anti-CTLA4 antibody comprises an HVR-H1 comprising the amino acid sequence of SEQ ID NO: 23, an HVR-H2 comprising the amino acid sequence of SEQ ID NO: 35, an HVR-H3 comprising the amino acid sequence of SEQ ID NO: 45, an HVR-L1 comprising the amino acid sequence of SEQ ID NO: 58, an HVR-L2 comprising the amino acid sequence of SEQ ID NO: 66, and an HVR-L3 comprising the amino acid sequence of SEQ ID NO: 75. In some of the foregoing embodiments, the anti-CTLA4 antibody comprises a heavy chain comprising the amino acid sequence of SEQ ID NO: 125 and a light chain comprising the amino acid sequence of SEQ ID NO: 127. In some of the foregoing embodiments, the anti-CTLA4 antibody comprises a heavy chain comprising the amino acid sequence of SEQ ID NO: 126 and a light chain comprising the amino acid sequence of SEQ ID NO: 127. In some of the foregoing embodiments, the anti-CTLA4 antibody is TY21580. In some of the foregoing embodiments, the anti CTLA4 antibody is administered to a patient with melanoma, non-small cell lung cancer, renal cell carcinoma, or hepatocellular carcinoma. In other of the foregoing embodiments, the anti CTLA4 antibody is administered to a patient with a MSI-H or dMMR cancer. In other of the foregoing embodiments, the anti CTLA4 antibody is administered to a patient with a cancer that has metastasized. In some of the foregoing embodiments, the anti-CTLA4 antibody is administered to a patient that is resistant or refractory to prior cancer therapy, including other anti-CTLA4 antibodies, anti-PD-1 antibodies, anti PD-L1 antibodies, or combinations thereof.

In some embodiments where the anti-CTLA4 antibody comprises a heavy chain variable region comprising the amino acid sequence of SEQ ID NO: 87 or an amino acid sequence having at least 90% sequence identity to the amino acid sequence of SEQ ID NO: 87, and/or a light chain variable region comprising the amino acid sequence of SEQ ID NO: 100 or an amino acid sequence having at least 90% sequence identity to the amino acid sequence of SEQ ID NO: 100, the anti-CTLA4 antibody is administered at a first higher dose (e.g., between about 10 mg/kg and about 20 mg) for at least one treatment cycle (as defined herein) followed by a lower dose (e.g., between about 0.5 mg/kg to about 10 mg/kg, about 0.5 mg/kg to about 6 mg/kg, or about 3 mg/kg to about 10 mg/kg) in subsequent cycles. In some such embodiments, the anti-CTLA4 antibody is administered at a dose of about 10 mg/kg for at least one treatment cycle (e.g., one to three treatment cycles) and at a dose of about 6 mg/kg in subsequent treatment cycles. In other such embodiments, the anti-CTLA4 antibody is administered at a dose of about 10 mg/kg for at least one treatment cycle (e.g., one to three treatment cycles) and at a dose of about 3 mg/kg in subsequent treatment cycles. In other such embodiments, the anti-CTLA4 antibody is administered at a dose of about 15 mg/kg for at least one treatment cycle (e.g., one to three treatment cycles) and at a dose of about 10 mg/kg in subsequent treatment cycles. In other such embodiments, the anti-CTLA4 antibody is administered at a dose of about 20 mg/kg for at least one treatment cycle (e.g., one to three treatment cycles) and at a dose of about 10 mg/kg in subsequent treatment cycles. In some of the foregoing embodiments, the anti-CTLA4 antibody comprises an HVR-H1 comprising the amino acid sequence of SEQ ID NO: 23, an HVR-H2 comprising the amino acid sequence of SEQ ID NO: 35, an HVR-H3 comprising the amino acid sequence of SEQ ID NO: 45, an HVR-L1 comprising the amino acid sequence of SEQ ID NO: 58, an HVR-L2 comprising the amino acid sequence of SEQ ID NO: 66, and an HVR-L3 comprising the amino acid sequence of SEQ ID NO: 75. In some of the foregoing embodiments, the anti-CTLA4 antibody comprises a heavy chain comprising the amino acid sequence of SEQ ID NO: 125 and a light chain comprising the amino acid sequence of SEQ ID NO: 127. In some of the foregoing embodiments, the anti-CTLA4 antibody comprises a heavy chain comprising the amino acid sequence of SEQ ID NO: 126 and a light chain comprising the amino acid sequence of SEQ ID NO: 127. In some of the foregoing embodiments, the anti-CTLA4 antibody is TY21580. In some of the foregoing embodiments, the anti CTLA4 antibody is administered to a patient with melanoma, non-small cell lung cancer, renal cell carcinoma, or hepatocellular carcinoma. In other of the foregoing embodiments, the anti CTLA4 antibody is administered to a patient with a MSI-H or dMMR cancer. In other of the foregoing embodiments, the anti CTLA4 antibody is administered to a patient with a cancer that has metastasized. In some of the foregoing embodiments, the anti-CTLA4 antibody is administered to a patient that is resistant or refractory to prior cancer therapy, including other anti-CTLA4 antibodies, anti-PD-1 antibodies, anti PD-L1 antibodies, or combinations thereof.

In some embodiments where the anti-CTLA4 antibody comprises a heavy chain variable region comprising the amino acid sequence of SEQ ID NO: 87 or an amino acid sequence having at least 90% sequence identity to the amino acid sequence of SEQ ID NO: 87, and/or a light chain variable region comprising the amino acid sequence of SEQ ID NO: 100 or an amino acid sequence having at least 90% sequence identity to the amino acid sequence of SEQ ID NO: 100, the anti-CTLA4 antibody is administered at a dose that provides a steady state concentration of from about 50 nM to about 700 nM in systemic circulation when measured at the last day of a dosing cycle (i.e., serum trough concentration). For instance, if the dosing cycle is once every three weeks, the serum trough concentration is measured on day 21 (i.e., after 504 hours) of the dosing cycle. If the dosing cycle is once every four weeks (i.e., after 672 hours), the serum trough concentration is measured on day 28 of the dosing cycle. In some embodiments, the anti-CTLA4 antibody is administered at a dose that provides a steady state serum trough concentration of from about 100 nM to about 500 nM. In other embodiments, the anti-CTLA4 antibody is administered at a dose that provides a steady state serum trough concentration of from about 100 nM to about 400 nM. In other embodiments, the anti-CTLA4 antibody is administered at a dose that provides a steady state serum trough concentration of from about 100 nM to about 200 nM. In other embodiments, the anti-CTLA4 antibody is administered at a dose that provides a steady state serum trough concentration of from about 150 nM to about 400 nM. In other embodiments, the anti-CTLA4 antibody is administered at a dose that provides a steady state serum trough concentration of from about 150 nM to about 450 nM. In some of the foregoing embodiments, the anti-CTLA4 antibody is administered once every three weeks. In some of the foregoing embodiments, the anti-CTLA4 antibody is administered once every four weeks. In some of the foregoing embodiments, the anti-CTLA4 antibody is administered once every two weeks. In some of the foregoing embodiments, the anti-CTLA4 antibody comprises an HVR-H1 comprising the amino acid sequence of SEQ ID NO: 23, an HVR-H2 comprising the amino acid sequence of SEQ ID NO: 35, an HVR-H3 comprising the amino acid sequence of SEQ ID NO: 45, an HVR-L1 comprising the amino acid sequence of SEQ ID NO: 58, an HVR-L2 comprising the amino acid sequence of SEQ ID NO: 66, and an HVR-L3 comprising the amino acid sequence of SEQ ID NO: 75. In some of the foregoing embodiments, the anti-CTLA4 antibody comprises a heavy chain comprising the amino acid sequence of SEQ ID NO: 125 and a light chain comprising the amino acid sequence of SEQ ID NO: 127. In some of the foregoing embodiments, the anti-CTLA4 antibody comprises a heavy chain comprising the amino acid sequence of SEQ ID NO: 126 and a light chain comprising the amino acid sequence of SEQ ID NO: 127. In some of the foregoing embodiments, the anti-CTLA4 antibody is TY21580. In some of the foregoing embodiments, the anti CTLA4 antibody is administered to a patient with melanoma, non-small cell lung cancer, renal cell carcinoma, or hepatocellular carcinoma. In other of the foregoing embodiments, the anti CTLA4 antibody is administered to a patient with a MSI-H or dMMR cancer. In other of the foregoing embodiments, the anti CTLA4 antibody is administered to a patient with a cancer that has metastasized. In some of the foregoing embodiments, the anti-CTLA4 antibody is administered to a patient that is resistant or refractory to prior cancer therapy, including other anti-CTLA4 antibodies, anti-PD-1 antibodies, anti PD-L1 antibodies, or combinations thereof.

In some embodiments where the anti-CTLA4 antibody comprises a heavy chain variable region comprising the amino acid sequence of SEQ ID NO: 87 or an amino acid sequence having at least 90% sequence identity to the amino acid sequence of SEQ ID NO: 87, and/or a light chain variable region comprising the amino acid sequence of SEQ ID NO: 100 or an amino acid sequence having at least 90% sequence identity to the amino acid sequence of SEQ ID NO: 100, the anti-CTLA4 antibody is administered at a dose that provides a concentration of from about 100 nM to about 2,000 nM in systemic circulation when measured after one week (i.e., 168 hours) of a dosing schedule. In some embodiments, the anti-CTLA4 antibody is administered at a dose that provides a concentration of from about 150 nM to about 1,500 nM in systemic circulation when measured after one week (i.e., 168 hours) of a dosing schedule. In other embodiments, the anti-CTLA4 antibody is administered at a dose that provides a concentration of from about 150 nM to about 1,000 nM in systemic circulation when measured after one week (i.e., 168 hours) of a dosing schedule. In other embodiments, the anti-CTLA4 antibody is administered at a dose that provides a concentration of from about 1,000 nM to about 2,000 nM in systemic circulation when measured after one week (i.e., 168 hours) of a dosing schedule. In other embodiments, the anti-CTLA4 antibody is administered at a dose that provides a concentration of from about 150 nM to about 500 nM in systemic circulation when measured after one week (i.e., 168 hours) of a dosing schedule. other embodiments, the anti-CTLA4 antibody is administered at a dose that provides a concentration of greater than about 150 nM when measured after one week (i.e., 168 hours) of a dosing schedule. In some of the foregoing embodiments, the anti-CTLA4 antibody is administered once every three weeks. In some of the foregoing embodiments, the anti-CTLA4 antibody is administered once every four weeks. In some of the foregoing embodiments, the anti-CTLA4 antibody is administered once every two weeks. In some of the foregoing embodiments, the anti-CTLA4 antibody comprises an HVR-H1 comprising the amino acid sequence of SEQ ID NO: 23, an HVR-H2 comprising the amino acid sequence of SEQ ID NO: 35, an HVR-H3 comprising the amino acid sequence of SEQ ID NO: 45, an HVR-L1 comprising the amino acid sequence of SEQ ID NO: 58, an HVR-L2 comprising the amino acid sequence of SEQ ID NO: 66, and an HVR-L3 comprising the amino acid sequence of SEQ ID NO: 75. In some of the foregoing embodiments, the anti-CTLA4 antibody comprises a heavy chain comprising the amino acid sequence of SEQ ID NO: 125 and a light chain comprising the amino acid sequence of SEQ ID NO: 127. In some of the foregoing embodiments, the anti-CTLA4 antibody comprises a heavy chain comprising the amino acid sequence of SEQ ID NO: 126 and a light chain comprising the amino acid sequence of SEQ ID NO: 127. In some of the foregoing embodiments, the anti-CTLA4 antibody is TY21580. In some of the foregoing embodiments, the anti CTLA4 antibody is administered to a patient with melanoma, non-small cell lung cancer, renal cell carcinoma, or hepatocellular carcinoma. In other of the foregoing embodiments, the anti CTLA4 antibody is administered to a patient with a MSI-H or dMMR cancer. In other of the foregoing embodiments, the anti CTLA4 antibody is administered to a patient with a cancer that has metastasized. In some of the foregoing embodiments, the anti-CTLA4 antibody is administered to a patient that is resistant or refractory to prior cancer therapy, including other anti-CTLA4 antibodies, anti-PD-1 antibodies, anti PD-L1 antibodies, or combinations thereof.

In some embodiments where the anti-CTLA4 antibody comprises a heavy chain variable region comprising the amino acid sequence of SEQ ID NO: 87 or an amino acid sequence having at least 90% sequence identity to the amino acid sequence of SEQ ID NO: 87, and/or a light chain variable region comprising the amino acid sequence of SEQ ID NO: 100 or an amino acid sequence having at least 90% sequence identity to the amino acid sequence of SEQ ID NO: 100, the anti-CTLA4 antibody is administered at a dose that results in a steady state AUC_{(0-∞)} of from about 500,000 day*ng/mL to about 2,500,000 day*ng/mL. In some embodiments, the anti-CTLA4 antibody is administered at a dose that results in a steady state AUC_{(0-∞)} of from about 500,000 day*ng/mL to about 1,500,000 day*ng/mL. In some embodiments, the anti-CTLA4 antibody is administered at a dose that results in a steady state AUC_{(0-∞)} of from about 500,000 day*ng/mL to about 1,000,000 day*ng/mL. In some embodiments, the anti-CTLA4 antibody is administered at a dose that results in a steady state AUC_{(0-∞)} of from about 500,000 day*ng/mL to about 1,000,000 day*ng/mL. In some embodiments, the anti-CTLA4 antibody is administered at a dose that results in a steady state AUC_{(0-∞)} of from about 1,500,000 day*ng/mL to about 2,000,000 day*ng/mL. In some embodiments, the anti-CTLA4 antibody is administered at a dose that results in a steady state AUC_{(0-∞)} of from about 2,000,000 day*ng/mL to about 2,500,000 day*ng/mL. In some embodiments, the steady state AUC_{(0-∞)} is achieved by administering the anti-CTLA4 antibody once every three weeks. In some embodiments, the steady state AUC_{(0-∞)} is achieved by administering the anti-CTLA4 antibody once every four weeks. In some embodiments, the steady state AUC_{(0-∞)} is achieved by administering the anti-CTLA4 antibody once every five weeks. In some embodiments, the steady state AUC_{(0-∞)} is achieved by administering the anti-CTLA4 antibody once every two weeks. In some of the foregoing embodiments, the anti-CTLA4 antibody comprises an HVR-H1 comprising the amino acid sequence of SEQ ID NO: 23, an HVR-H2 comprising the amino acid sequence of SEQ ID NO: 35, an HVR-H3 comprising the amino acid sequence of SEQ ID NO: 45, an HVR-L1 comprising the amino acid sequence of SEQ ID NO: 58, an HVR-L2 comprising the amino acid sequence of SEQ ID NO: 66, and an HVR-L3 comprising the amino acid sequence of SEQ ID NO: 75. In some of the foregoing embodiments, the anti-CTLA4 antibody comprises a heavy chain comprising the amino acid sequence of SEQ ID NO: 125 and a light chain comprising the amino acid sequence of SEQ ID NO: 127. In some of the foregoing embodiments, the anti-CTLA4 antibody comprises a heavy chain comprising the amino acid sequence of SEQ ID NO: 126 and a light chain comprising the amino acid sequence of SEQ ID NO: 127. In some of the foregoing embodiments, the anti-CTLA4 antibody is TY21580. In some of the foregoing embodiments, the anti CTLA4 antibody is administered to a patient with melanoma, non-small cell lung cancer, renal cell carcinoma, or hepatocellular carcinoma. In other of the foregoing embodiments, the anti CTLA4 antibody is administered to a patient with a MSI-H or dMMR cancer. In other of the foregoing embodiments, the anti CTLA4 antibody is administered to a patient with a cancer that has metastasized. In some of the foregoing embodiments, the anti-CTLA4 antibody is administered to a patient that is resistant or refractory to prior cancer therapy, including other anti-CTLA4 antibodies, anti-PD-1 antibodies, anti PD-L1 antibodies, or combinations thereof.

The effective amount of the anti-CTLA4 antibody may be administered in a single dose or in multiple doses. For methods that comprises administration of the anti-CTLA4 antibody in multiple doses, exemplary dosing frequencies include, but are not limited to, weekly, weekly without break, weekly for two out of three weeks, weekly for three out of four weeks, once every three weeks, once every two weeks, monthly, every six months, yearly, etc. In some embodiments, the anti-CTLA4 antibody is administered about weekly, once every 2 weeks, once every 3 weeks, once every 6 weeks, or once every 12 weeks. In some embodiments, the intervals between each administration are less than about any of 3 years, 2 years, 12 months, 11 months, 10 months, 9 months, 8 months, 7 months, 6 months, 5 months, 4 months, 3 months, 2 months, 1 month, 4 weeks, 3 weeks, 2 weeks, or 1 week. In some embodiments, the intervals between each administration are more than about any of 1 week, 2 weeks, 3 weeks, 4 weeks, 1 month, 2 months, 3 months, 4 months, 5 months, 6 months, 7 months, 8 months, 9 months, 10 months, 11 months, 12 months, 2 years, or 3 years. In some embodiments, there is no break in the dosing schedule.

In some embodiments, the anti-CTLA4 antibody is administered at a low frequency, for example, any one of no more frequent than once per week, once every other week, once per three weeks, once per month, once per 2 months, once per 3 months, once per 4 months, once per 5 months, once per 6 months, once per 7 months, once per 8 months, once per 9 months, once per 10 months, once per 11 months, once per year, or less. In some embodiments, the anti-CTLA4 antibody is administered in a single dose. In some embodiments, the anti-CTLA4 antibody is administered about once every three weeks.

In some embodiments, the anti-CTLA4 antibody is administered for 2 or more cycles, such as about any one of 2, 3, 4, 5, 6, 7, 8, 9, 10, 11, 12, or more cycles. In some embodiments, the anti-CTLA4 antibody is administered for at least 4 cycles.

In some embodiments, the treatment comprises an initial phase and a subsequent maintenance phase. In some embodiments, the anti-CTLA4 antibody is administered less frequently in the maintenance phase than in the initial phase. In some embodiments, the anti-CTLA4 antibody is administered at the same frequently in the maintenance phase as in the initial phase. In some embodiments, the treatment comprises an initial phase wherein the anti-CTLA4 antibody is administered about once every three weeks for at least 4 cycles, and a maintenance phase wherein the anti-CTLA4 antibody is administered about once every 4 weeks to once every 12 weeks, such as once every 4 weeks, once every 6 weeks, once every 8 weeks, once every 10 weeks, or once every 12 weeks. In some embodiments, the dosing frequency in the maintenance phase is adjusted depending on one or more biomarkers, such as T_{reg} cells, CD8+ Tₑₘ cells, CD4+Tₑₘ cells, a ratio of CD8+ Tₑₘ cells to T_{reg} cells, a ratio of CD4+ Tₑₘ cells to T_{reg} cells, and/or NK cells. For example, if the subject shows an increase in the ratio of CD8+ Tₑₘ cells to T_{reg} cells after receiving the anti-CTLA4 antibody, the subject may be further administered an anti-CTLA4 antibody at about every 4 weeks.

The administration of the anti-CTLA4 antibody can be extended over an extended period of time, such as from about a week to about a month, from about a month to about a year, from about a year to about several years. In some embodiments, the anti-CTLA4 antibody is administered over a period of at least any of about 1 week, 2 weeks, 3 weeks, 4 weeks, 5 weeks, 1 month, 2 months, 3 months, 4 months, 5 months, 6 months, 7 months, 8 months, 9 months, 10 months, 11 months, 12 months, 1 year, 2 years, 3 years, 4 years, or more.

The methods described herein are useful for treating a variety of cancers. In some embodiments, the cancer is a solid cancer. In some embodiments, the cancer is a liquid cancer. A variety of cancers where CTLA4 is implicated, whether malignant or benign and whether primary or secondary, may be treated or prevented with a method provided by the disclosure. Exemplary cancers include, but are not limited to, liver cancer, a cancer of the digestive system (e.g., colon cancer, colorectal cancer), lung cancer, bone cancer, heart cancer, brain cancer, kidney cancer, bladder cancer, a hematological cancer (e.g., leukemia), skin cancer, breast cancer, thyroid cancer, pancreatic cancer, a head and/or neck cancer, an eye-related cancer, a male reproductive system cancer (e.g., prostate cancer, testicular cancer), or a female reproductive system cancer (e.g., uterine cancer, cervical cancer). In some embodiments, the cancer is kidney cancer, such as renal cell carcinoma, or urothelial carcinoma.

In some embodiments, the subject has been previously treated with a prior therapy. In some embodiments, the subject has previously received any one of 1, 2, 3, 4, or more prior therapies. In some embodiments, the subject has exhausted all other available therapies. In some embodiments, the subject is unresponsive or resistant to a prior therapy. In some embodiments, the subject has disease reoccurrence subsequent to a prior therapy. In some embodiments, the subject is refractory to a prior therapy. In some embodiments, the subject has failed a prior therapy within about 1 year, 6 months, 3 months or less. In some embodiments, the subject has not previously received a prior therapy.

In some embodiments, the subject has been previously treated with a standard therapy for the cancer. In some embodiments, the subject is unresponsive or resistant to a standard therapy. In some embodiments, the subject has disease reoccurrence subsequent to a standard therapy. In some embodiments, the subject is refractory to a standard therapy. In some embodiments, the subject has failed a standard therapy within about 1 year, 6 months, 3 months or less. In some embodiments, the subject has not previously received a standard therapy. In some embodiments, the subject has refused or is ineligible for a standard therapy.

In some embodiments, the prior therapy (e.g., standard therapy) is selected from the group consisting of viral gene therapy, immunotherapy, targeted therapy, radiation therapy, and chemotherapy. In some embodiments, the prior therapy is an immune checkpoint inhibitor. In some embodiments, the prior therapy is an inhibitor of CTLA4, PD-1, or a PD-1 ligand (e.g., PD-L1 or PD-L2). In some embodiments, the prior therapy is an inhibitor of CTLA4, such as an anti-CTLA4 antibody that is different from the anti-CTLA4 antibodies described herein. In some embodiments, the prior therapy is ipilimumab. In some embodiments, the prior therapy is an inhibitor of CTLA4 (e.g., an anti-CTLA4 antibody) and an inhibitor of PD-1 (e.g., an anti-PD-1 antibody). In some embodiments, the prior therapy is an inhibitor of CTLA4 (e.g., an anti-CTLA4 antibody) and an inhibitor of PD-1 ligand (e.g., an anti-PD-L1 antibody or an anti-PD-L2 antibody).

In some embodiments, the prior therapy is an inhibitor of PD-1 or a PD-1 ligand, including a PD-1 binding antagonist, a PDL1 binding antagonist and a PDL2 binding antagonist. Alternative names for “PD-1” include CD279 and SLEB2. Alternative names for “PDL1” include B7-H1, B7-4, CD274, and B7-H. Alternative names for “PDL2” include B7-DC, Btdc, and CD273. In some embodiments, PD-1, PDL1, and PDL2 are human PD-1, PDL1 and PDL2.

In some embodiments, the inhibitor of PD-1 is a molecule that inhibits the binding of PD-1 to its ligand binding partners. In some embodiments, the inhibitor of a PD-1 ligand is an inhibitor of PD-L1 and/or PD-L2. In some embodiments, the inhibitor of PD-L1 is a molecule that inhibits the binding of PDL1 to its binding partners. In some embodiments, a PD-L2 binding partner is PD-1 and/or B7-1. In some embodiments, the inhibitor of a PD-1 ligand is a molecule that inhibits the binding of PD-L2 to its binding partners. In some embodiments, a PD-L2 binding partner is PD-1. The inhibitor may be an antibody, an antigen binding fragment thereof, an immunoadhesin, a fusion protein, or oligopeptide.

In some embodiments, the inhibitor of PD-1 is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the inhibitor of PD-1 is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the inhibitor of PD-1 is AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PDL2-Fc fusion soluble receptor described in WO2010/027827 and WO2011/066342. In some embodiments, the anti-PD-1 antibody is nivolumab (CAS Registry Number: 946414-94-4). In some embodiments, the anti-PD-1 antibody is pembrolizumab (CAS Registry Number: 1374853-91-4).

In some embodiments, the inhibitor of PD-L1 is anti-PD-L1 antibody. In some embodiments, the inhibitor of PD-L1 is selected from the group consisting of YW243.55.S70, MPDL3280A, MDX-1105, and MEDI4736. MDX-1105, also known as BMS-936559, is an anti-PD-L1 antibody described in WO2007/005874. Antibody YW243.55.S70 (heavy and light chain variable region sequences shown in SEQ ID Nos. 20 and 21, respectively) is an anti-PD-L1 described in WO 2010/077634 A1. MEDI4736 is an anti-PD-L1 antibody described in WO2011/066389 and US2013/034559. Examples of anti-PD-L1 antibodies useful for the methods of this application, and methods for making thereof are described in PCT patent application WO 2010/077634 A1 and U.S. Pat. No. 8,217,149, which are incorporated herein by reference.

Prior therapies (e.g., standard therapies) also encompass surgery to remove a tumor and radiation therapy. Exemplary radiation therapies include, but are not limited to, ionizing (electromagnetic) radiotherapy (e.g., X-rays or gamma rays) and particle beam radiation therapy (e.g., high linear energy radiation). The source of radiation can be external or internal to the subject.

The methods described herein are useful for various aspects of cancer treatment. In some embodiments, there is provided a method of inhibiting cell proliferation (such as tumor growth) in an individual, comprising administering to the individual an effective amount of any one of the anti-CTLA4 antibodies described herein. In some embodiments, at least about 10% (including for example at least about any of 20%, 30%, 40%, 60%, 70%, 80%, 90%, 95% or more) cell proliferation is inhibited.

In some embodiments, there is provided a method of inhibiting tumor metastasis in an individual, comprising administering to the individual an effective amount of any one of the anti-CTLA4 antibodies described herein. In some embodiments, at least about 10% (including for example at least about any of 20%, 30%, 40%, 60%, 70%, 80%, 90%, 95% or more) metastasis is inhibited.

In some embodiments, there is provided a method of reducing (such as eradicating) pre-existing tumor metastasis (such as metastasis to the lymph node) in an individual, comprising administering to the individual an effective amount of any one of the anti-CTLA4 antibodies described herein. In some embodiments, at least about 10% (including for example at least about any of 20%, 30%, 40%, 60%, 70%, 80%, 90%, 95% or more) metastasis is reduced.

In some embodiments, there is provided a method of reducing incidence or burden of preexisting tumor metastasis (such as metastasis to the lymph node) in an individual, comprising administering to the individual an effective amount of any one of the anti-CTLA4 antibodies described herein.

In some embodiments, there is provided a method of reducing tumor size in an individual, comprising administering to the individual an effective amount of any one of the anti-CTLA4 antibodies described herein. In some embodiments, the method reduces tumor size by at least about 100 (including for example at least about any of 20%, 30%, 40%, 60%, 70%, 80%, 90%, 95% or more).

In some embodiments, there is provided a method of prolonging time to disease progression of cancer in an individual, comprising administering to the individual an effective amount of any one of the anti-CTLA4 antibodies described herein. In some embodiments, the method prolongs the time to disease progression by at least any of 1, 2, 3, 4, 5, 6, 7, 8, 9, 10, 11, 12, 16, 20, 24, 28, 32, 36, or more weeks.

In some embodiments, there is provided a method of prolonging survival (e.g., overall survival or progression-free survival) of an individual having cancer, comprising administering to the individual an effective amount of any one of the anti-CTLA4 antibodies described herein. In some embodiments, the method prolongs the survival of the individual by at least any of 1, 2, 3, 4, 5, 6, 7, 8, 9, 10, 11, 12, 18, or 24 months.

In some embodiments, there is provided a method of alleviating one or more symptoms in an individual having cancer, comprising administering to the individual an effective amount of any one of the anti-CTLA4 antibodies described herein.

In some embodiments, there is provided a method of improving the quality of life in an individual having cancer, comprising administering to the individual an effective amount of any one of the anti-CTLA4 antibodies described herein.

The anti-CTLA4 antibody may be administered alone as monotherapy, or administered in combination with one or more additional therapeutic agents or therapies. In some embodiment, anti-CTLA4 antibody is administered in combination with one or more additional therapeutic agents for separate, sequential or simultaneous administration. The term “additional therapeutic agent” refers to any therapeutic agent other than an anti-CTLA4 antibody provided by the disclosure. In some embodiments, there is provided a combination therapy for treating cancer in a subject, which comprises administering to the subject a therapeutically effective amount of an anti-CTLA4 antibody described herein in combination with one or more additional therapeutic agents. In some embodiments, anti-CTLA4 antibody is administered in combination with one or more additional therapeutic agents comprising chemotherapeutic agents, immunotherapeutic agents, and/or hormone therapeutic agents. In some embodiments, the one or more additional therapeutic agents are selected from the group consisting of selected from the group consisting of viral gene therapy, immune checkpoint inhibitors, targeted therapies, radiation therapies, and chemotherapies.

The term “chemotherapeutic agent” refers to a chemical or biological substance that can cause death of cancer cells, or interfere with growth, division, repair, and/or function of cancer cells. Examples of chemotherapeutic agents include those that are disclosed in WO 2006/129163, and US 20060153808, the disclosures of which are incorporated herein by reference. Examples of particular chemotherapeutic agents include: (1) alkylating agents, such as chlorambucil (LEUKERAN®), mcyclophosphamide (CYTOXAN®), ifosfamide (IFEX®), mechlorethamine hydrochloride (MUSTARGEN), thiotepa (THIOPLEX®), streptozotocin (ZANOSAR®), carmustine (BICNU®, GLIADEL WAFER®), lomustine (CEENU®), and dacarbazine (DTIC-DOME®); (2) alkaloids or plant vinca alkaloids, including cytotoxic antibiotics, such as doxorubicin (ADRIAMYCIN®), epirubicin (ELLENCE®, PHARMORUBICIN®), daunorubicin (CERUBIDINE®, DAUNOXOME®), nemorubicin, idarubicin (IDAMYCIN PFS®, ZAVEDOS®), mitoxantrone (DHAD®, NOVANTRONE®), dactinomycin (actinomycin D, COSMEGEN®), plicamycin (MITHRACIN®), mitomycin (MUTAMYCIN®), and bleomycin (BLENOXANE®), vinorelbine tartrate (NAVELBINE®), vinblastine (VELBAN®), vincristine (ONCOVIN®), and vindesine (ELDISINE®); (3) antimetabolites, such as capecitabine (XELODA®), cytarabine (CYTOSAR-U®), fludarabine (FLUDARA®), gemcitabine (GEMZAR®), hydroxyurea (HYDRA®), methotrexate (FOLEX®, MEXATE, TREXALL®), nelarabine (ARRANON®), trimetrexate (NEUTREXIN®), and pemetrexed (ALIMTA®); (4) Pyrimidine antagonists, such as 5-fluorouracil (5-FU); capecitabine (XELODA®), raltitrexed (TOMUDEX®), tegafur-uracil (UFTORAL®), and gemcitabine (GEMZAR®); (5) taxanes, such as docetaxel (TAXOTERE®), paclitaxel (TAXOL®); (6) platinum drugs, such as cisplatin (PLATINOL®) and carboplatin (PARAPLATIN®), and oxaliplatin (ELOXATIN®); (7) topoisomerase inhibitors, such as irinotecan (CAMPTOSAR®), topotecan (HYCAMTIN®), etoposide (ETOPOPHOS®, VEPESSID®, TOPOSAR®), and teniposide (VUMON®); (8) epipodophyllotoxins (podophyllotoxin derivatives), such as etoposide (ETOPOPHOS®, VEPESSID®, TOPOSAR®); (9) folic acid derivatives, such as leucovorin (WELLCOVORIN®); (10) nitrosoureas, such as carmustine (BICNU®), lomustine (CEENU®); (11) inhibitors of receptor tyrosine kinase, including epidermal growth factor receptor (EGFR), vascular endothelial growth factor (VEGF), insulin receptor, insulin-like growth factor receptor (IGFR), hepatocyte growth factor receptor (HGFR), and platelet-derived growth factor receptor (PDGFR), such as gefitinib (IRESSA®), erlotinib (TARCEVA®), bortezomib (VELCADE®), imatinib mesylate (GLEEVEC®), genefitinib, lapatinib, sorafenib, thalidomide, sunitinib (SUTENT®), axitinib, rituximab (RITUXAN, MABTHERA®), trastuzumab (HERCEPTIN®), cetuximab (ERBITUX®), bevacizumab (AVASTIN®), and ranibizumab (LUCENTIS®), lym-1 (ONCOLYM®), antibodies to insulin-like growth factor-1 receptor (IGF-1R) that are disclosed in WO2002/053596); (12) angiogenesis inhibitors, such as bevacizumab (AVASTIN®), suramin (GERMANIN®), angiostatin, SU5416, thalidomide, and matrix metalloproteinase inhibitors (such as batimastat and marimastat), and those that are disclosed in WO2002055106; and (13) proteasome inhibitors, such as bortezomib (VELCADE®).

The term “immunotherapeutic agents” refers to a chemical or biological substance that can enhance an immune response of a mammal. Examples of immunotherapeutic agents include: bacillus Calmette-Guerin (BCG); cytokines such as interferons; vaccines such as MyVax personalized immunotherapy, Onyvax-P, Oncophage, GRNVAC1, Favld, Provenge, GVAX, Lovaxin C, BiovaxID, GMXX, and NeuVax; and antibodies such as alemtuzumab (CAMPATH®), bevacizumab (AVASTIN®), cetuximab (ERBITUX®), gemtuzunab ozogamicin (MYLOTARG®), ibritumomab tiuxetan (ZEVALIN®), panitumumab (VECTIBIX®), rituximab (RITUXAN®, MABTHERA®), trastuzumab (HERCEPTIN®), tositumomab (BEXXAR®), ipilimumab (YERVOY®), tremelimumab, CAT-3888, agonist antibodies to OX40 receptor (such as those disclosed in WO2009/079335), agonist antibodies to CD40 receptor (such as those disclosed in WO2003/040170, and TLR-9 agonists (such as those disclosed in WO2003/015711, WO2004/016805, and WO2009/022215).

The term “hormone therapeutic agent” refers to a chemical or biological substance that inhibits or eliminates the production of a hormone, or inhibits or counteracts the effect of a hormone on the growth and/or survival of cancerous cells. Examples of such agents suitable for the methods herein include those that are disclosed in US20070117809. Examples of particular hormone therapeutic agents include tamoxifen (NOLVADEX®), toremifene (FARESTON®), fulvestrant (FASLODEX®), anastrozole (ARIMIDEX®), exemestane (AROMASIN®), letrozole (FEMARA®), megestrol acetate (MEGACE®), goserelin (ZOLADEX®), and leuprolide (LUPRON®). The anti-CTLA4 antibodies of this disclosure may also be used in combination with non-drug hormone therapies such as (1) surgical methods that remove all or part of the organs or glands which participate in the production of the hormone, such as the ovaries, the testicles, the adrenal gland, and the pituitary gland, and (2) radiation treatment, in which the organs or glands of the patient are subjected to radiation in an amount sufficient to inhibit or eliminate the production of the targeted hormone.

In some embodiments, the additional therapeutic agent is one or more of pomalyst, revlimid, lenalidomide, pomalidomide, thalidomide, a DNA-alkylating platinum-containing derivative, cisplatin, 5-fluorouracil, cyclophosphamide, an anti-CD137 antibody, an anti-PD-1 antibody, an anti-PD-L1 antibody, an anti-CD20 antibody, an anti-CD40 antibody, an anti-DR5 antibody, an anti-CD1d antibody, an anti-TIM3 antibody, an anti-SLAMF7 antibody, an anti-KIR receptor antibody, an anti-OX40 antibody, an anti-HER2 antibody, an anti-ErbB-2 antibody, an anti-EGFR antibody, cetuximab, rituximab, trastuzumab, pembrolizumab, radiotherapy, single dose radiation, fractionated radiation, focal radiation, whole organ radiation, IL-12, IFNα, GM-CSF, a chimeric antigen receptor, adoptively transferred T cells, an anti-cancer vaccine, and an oncolytic virus. In some embodiments, the additional therapeutic agent is an anti-PD-1 antibody. In some embodiments, the additional therapeutic agent is an anti-PD-1 antibody.

The combination therapy for treating cancer also encompasses the combination of an anti-CTLA4 antibody with surgery to remove a tumor. The anti-CTLA4 antibody may be administered to the mammal before, during, or after the surgery.

The combination therapy for treating cancer also encompasses combination of an anti-CTLA4 antibody with radiation therapy, such as ionizing (electromagnetic) radiotherapy (e.g., X-rays or gamma rays) and particle beam radiation therapy (e.g., high linear energy radiation). The source of radiation can be external or internal to the mammal. The anti-CTLA4 antibody may be administered to the mammal before, during, or after the radiation therapy.

Also provided are compositions of any one of the anti-CTLA4 antibodies described herein for use in the methods described in this section, and use of the anti-CTLA4 antibodies in the manufacture of a medicament for treating cancer (such as a solid cancer, e.g., urothelial carcinoma).

### III. Biomarkers

The present application also provides biomarkers, which can be used in conjunction with any one of the methods of treatment described herein. Suitable biomarkers include IL-1β, IL-2, IL-6, interferon (IFN)-γ, tumor necrosis factor (TNF)-α, soluble CTLA4 (sCTLA4), soluble PD-L1 (sPD-L1), soluble CD25 (sCD25), CXCL11, FoxP3, Ki67, CD8+ T cells, CD4+ T cells, CD8+ effector memory T (Tₑₘ) cells, CD4+ Tₑₘ cells, regulatory T (T_{reg}) cells, a ratio of CD8+ Tₑₘ cells to T_{reg} cells, a ratio of CD4+ Tₑₘ cells to T_{reg} cells, NK cells and B cells.

In some embodiments, there is provided a method of treating or delaying progression of cancer in a subject by administering an effective amount of an anti-CTLA4 antibody based on a level of one or more biomarkers selected from the group consisting of IL-10, IL-2, IL-6, IL-10, IFN-γ, TNF-α, sCTLA4, sPD-L1, sCD25, CXCL11, FoxP3, Ki67, CD8+ T cells, CD4+ T cells, CD8+ Tₑₘ cells, CD4+ Tₑₘ cells, T_{reg} cells, a ratio of CD8+ Tₑₘ cells to T_{reg} cells, a ratio of CD4+Tₑₘ cells to T_{reg} cells, NK cells and B cells in one or more samples obtained from the subject. In some embodiments, the one or more biomarkers comprise CD8+ T cells, CD4+ T cells, CD8+ Tₑₘ cells, CD4+ Tₑₘ cells, T_{reg} cells, a ratio of CD8+ Tₑₘ cells to T_{reg} cells, a ratio of CD4+ Tₑₘ cells to T_{reg} cells, NK cells and B cells. In some embodiments, the one or more biomarkers comprises CD8+ Tₑₘ cells. In some embodiments, the one or more biomarkers comprises CD4+ Tₑₘ cells. In some embodiments, the one or more biomarkers comprises T_{reg} cells. In some embodiments, the one or more biomarkers comprises a ratio of CD8+ Tₑₘ cells to T_{reg} cells. In some embodiments, the one or more biomarkers comprises a ratio of CD4+ Tₑₘ cells to T_{reg} cells. In some embodiments, the one or more biomarkers comprises NK cells.

In some embodiments, there is provided a method of determining whether a subject is likely to respond to a therapy comprising an anti-CTLA4 antibody by determining a level of one or more biomarkers selected from the group consisting of IL-1β, IL-2, IL-6, IL-10, IFN-γ, TNF-α, sCTLA4, sPD-L1, sCD25, CXCL11, FoxP3, Ki67, CD8+ T cells, CD4+ T cells, CD8+ Tₑₘ cells, CD4+ T_{reg} cells, T_{reg} cells, a ratio of CD8+ Tₑₘ cells to T_{reg} cells, a ratio of CD4+ Tₑₘ cells to T_{reg} cells, NK cells and B cells in one or more samples obtained from the subject. In some embodiments, there is provided a method of treating or delaying progression of cancer in a subject by administering an effective amount of an anti-CTLA4 antibody after it has been determined that the subject is likely to respond to the anti-CTLA4 antibody. In some embodiments, the one or more biomarkers comprise CD8+ T cells, CD4+ T cells, CD8+ Tₑₘ cells, CD4+ Tₑₘ cells, T_{reg} cells, a ratio of CD8+ Tₑₘ cells to T_{reg} cells, a ratio of CD4+ Tₑₘ cells to T_{reg} cells, NK cells and B cells. In some embodiments, the one or more biomarkers comprises CD8+ Tₑₘ cells. In some embodiments, the one or more biomarkers comprises CD4+ Tₑₘ cells. In some embodiments, the one or more biomarkers comprises T_{reg} cells. In some embodiments, the one or more biomarkers comprises a ratio of CD8+ Tₑₘ cells to T_{reg} cells. In some embodiments, the one or more biomarkers comprises a ratio of CD4+ Tₑₘ cells to Treg cells. In some embodiments, the one or more biomarkers comprises NK cells.

In some embodiments, there is provided a method of selecting a subject to receive or not to receive a therapy comprising an anti-CTLA4 antibody based on a level of one or more biomarkers selected from the group consisting of IL-1β, IL-2, IL-6, IL-10, IFN-γ, TNF-α, sCTLA4, sPD-L1, sCD25, CXCL11, FoxP3, Ki67, CD8+ T cells, CD4+ T cells, CD8+ Tₑₘ cells, CD4+ Tₑₘ cells, T_{reg} cells, a ratio of CD8+ Tₑₘ cells to Treg cells, a ratio of CD4+ Tₑₘ cells to T_{reg} cells, NK cells and B cells in one or more samples obtained from the subject. In some embodiments, the one or more biomarkers comprise CD8+ T cells, CD4+ T cells, CD8+ Tₑₘ cells, CD4+ Tₑₘ cells, T_{reg} cells, a ratio of CD8+ Tₑₘ cells to T_{reg} cells, a ratio of CD4+ Tₑₘ cells to T_{reg} cells, NK cells and B cells. In some embodiments, the one or more biomarkers comprises CD8+Tₑₘ cells. In some embodiments, the one or more biomarkers comprises CD4+ Tₑₘ cells. In some embodiments, the one or more biomarkers comprises T_{reg} cells. In some embodiments, the one or more biomarkers comprises a ratio of CD8+ Tₑₘ cells to T_{reg} cells. In some embodiments, the one or more biomarkers comprises a ratio of CD4+ Tₑₘ cells to T_{reg} cells. In some embodiments, the one or more biomarkers comprises NK cells.

In some embodiments, there is provided a method of predicting responsiveness and/or monitoring treatment and/or responsiveness of a subject to a therapy comprising an anti-CTLA4 antibody by determining a level of one or more biomarkers selected from the group consisting of IL-1β, IL-2, IL-6, IL-10, IFN-γ, TNF-α, sCTLA4, sPD-L1, sCD25, CXCL11, FoxP3, Ki67, CD8+ T cells, CD4+ T cells, CD8+ Tₑₘ cells, CD4+ Tₑₘ cells, T_{reg} cells, a ratio of CD8+ Tₑₘ cells to T_{reg} cells, a ratio of CD4+ Tₑₘ cells to T_{reg} cells, NK cells and B cells in one or more samples obtained from the subject. In some embodiments, the one or more biomarkers comprise CD8+ T cells, CD4+ T cells, CD8+ Tₑₘ cells, CD4+ Tₑₘ cells, T_{reg} cells, a ratio of CD8+ Tₑₘ cells to T_{reg} cells, a ratio of CD4+ Tₑₘ cells to T_{reg} cells, NK cells and B cells. In some embodiments, the one or more biomarkers comprises CD8+ Tₑₘ cells. In some embodiments, the one or more biomarkers comprises CD4+ Tₑₘ cells. In some embodiments, the one or more biomarkers comprises T_{reg} cells. In some embodiments, the one or more biomarkers comprises a ratio of CD8+ Tₑₘ cells to T_{reg} cells. In some embodiments, the one or more biomarkers comprises a ratio of CD4+ Tₑₘ cells to Treg cells. In some embodiments, the one or more biomarkers comprises NK cells.

In some embodiments, there is provided a method of positively and/or negatively stratifying patients into particular treatment regimen groups based upon a level of one or more biomarkers selected from the group consisting of IL-1β, IL-2, IL-6, IL-10, IFN-γ, TNF-α, sCTLA4, sPD-L1, sCD25, CXCL11, FoxP3, Ki67, CD8+ T cells, CD4+ T cells, CD8+ Tₑₘ cells, CD4+ Tₑₘ cells, T_{reg} cells, a ratio of CD8+ Tₑₘ cells to Treg cells, a ratio of CD4+ Tₑₘ cells to T_{reg} cells, NK cells and B cells in one or more samples obtained from the patients. In some embodiments, the treatment comprises administration of an anti-CTLA4 antibody. In some embodiments, the one or more biomarkers comprise CD8+ T cells, CD4+ T cells, CD8+ Tₑₘ cells, CD4+ Tₑₘ cells, T_{reg} cells, a ratio of CD8+ Tₑₘ cells to T_{reg} cells, a ratio of CD4+ Tₑₘ cells to T_{reg} cells, NK cells and B cells. In some embodiments, the one or more biomarkers comprises CD8+Tₑₘ cells. In some embodiments, the one or more biomarkers comprises CD4+ Tₑₘ cells. In some embodiments, the one or more biomarkers comprises T_{reg} cells. In some embodiments, the one or more biomarkers comprises a ratio of CD8+ Tₑₘ cells to T_{reg} cells. In some embodiments, the one or more biomarkers comprises a ratio of CD4+ Tₑₘ cells to T_{reg} cells. In some embodiments, the one or more biomarkers comprises NK cells.

In some embodiments, there is provided an assay for determining a level of one or more biomarkers selected from the group consisting of IL-1β, IL-2, IL-6, IL-10, IFN-γ, TNF-α, sCTLA4, sPD-L1, sCD25, CXCL11, FoxP3, Ki67, CD8+ T cells, CD4+ T cells, CD8+ Tₑₘ cells, CD4+ Tₑₘ cells, T_{reg} cells, a ratio of CD8+ Tₑₘ cells to T_{reg} cells, a ratio of CD4+ Tₑₘ cells to T_{reg} cells, NK cells and B cells in one or more samples obtained from the subject. In some embodiments, the assay is a flow cytometry assay. In some embodiments, the assay is an immunohistochemistry (IHC) assay. In some embodiments, the assay is a multiplex IHC assay capable of detecting two or more biomarkers. In some embodiments, the assay is an immunoassay, such as a Meso Scale Discovery (MSD) assay. In some embodiments, the one or more biomarkers comprise CD8+ T cells, CD4+ T cells, CD8+ Tₑₘ cells, CD4+ Tₑₘ cells, T_{reg} cells, a ratio of CD8+Tₑₘ cells to T_{reg} cells, a ratio of CD4+ Tₑₘ cells to T_{reg} cells, NK cells and B cells. In some embodiments, the one or more biomarkers comprises CD8+ Tₑₘ cells. In some embodiments, the one or more biomarkers comprises CD4+ Tₑₘ cells. In some embodiments, the one or more biomarkers comprises T_{reg} cells. In some embodiments, the one or more biomarkers comprises a ratio of CD8+ Tₑₘ cells to T_{reg} cells. In some embodiments, the one or more biomarkers comprises a ratio of CD4+ Tₑₘ cells to T_{reg} cells. In some embodiments, the one or more biomarkers comprises NK cells.

In some embodiments, there is provided a method of treating a cancer in a subject, comprising: (a) administering to the subject an effective amount of an anti-CTLA4 antibody, wherein the antibody specifically binds to an epitope comprising amino acid residues Y105 and L106 of human CTLA4 but does not comprise residue I108, wherein the numbering of the amino acid residues is according to SEQ ID NO: 108, (b) subsequently determining a level of one or more biomarkers selected from the group consisting of CD8+ T cells, CD4+ T cells, CD8+ Tₑₘ cells, CD4+ Tₑₘ cells, T_{reg} cells, a ratio of CD8+ Tₑₘ cells to T_{reg} cells, a ratio of CD4+ Tₑₘ cells to T_{reg} cells, NK cells, B cells in a sample of the subject. In some embodiments, an increase of the level of one or more biomarkers selected from the group consisting of CD8+ T cells, CD4+ T cells, CD8+ Tₑₘ cells, CD4+ Tₑₘ cells, a ratio of CD8+ Tₑₘ cells to T_{reg} cells, a ratio of CD4+ Tₑₘ cells to T_{reg} cells, NK cells and B cells after administration of the anti-CTLA4 antibody compared to the baseline level of the one or more biomarkers indicates an increased likelihood that the subject has an effective response to the CTLA4 antibody. In some embodiments, a decrease of the level of T_{reg} cells after administration of the anti-CTLA4 antibody compared to the baseline level of the T_{reg} cells indicates an increased likelihood that the subject has an effective response to the CTLA4 antibody. In some embodiments, the cancer is a solid cancer, such as advanced-stage and/or metastatic cancer. In some embodiments, the cancer is urothelial carcinoma. In some embodiments, the cancer is resistant or refractory to a prior therapy, wherein the prior therapy is an inhibitor of CTLA4 (e.g., ipilimumab), PD-1 (e.g., pembrolizumab), or a PD-1 ligand. In some embodiments, the one or more biomarkers comprises CD8+ Tₑₘ cells. In some embodiments, the one or more biomarkers comprises CD4+ Tₑₘ cells. In some embodiments, the one or more biomarkers comprises T_{reg} cells. In some embodiments, the one or more biomarkers comprises a ratio of CD8+ Tₑₘ cells to T_{reg} cells. In some embodiments, the one or more biomarkers comprises a ratio of CD4+ Tₑₘ cells to T_{reg} cells. In some embodiments, the one or more biomarkers comprises NK cells. In some embodiments, the antibody is TY21580.

In some embodiments, there is provided a method of treating a cancer in a subject, comprising: (a) administering to the subject an effective amount of an anti-CTLA4 antibody, wherein the antibody specifically binds to an epitope comprising amino acid residues Y105 and L106 of human CTLA4 but does not comprise residue I108, wherein the numbering of the amino acid residues is according to SEQ ID NO: 108; (b) subsequently determining a level of one or more biomarkers selected from the group consisting of CD8+ T cells, CD4+ T cells, CD8+ Tₑₘ cells, CD4+ Tₑₘ cells, T_{reg} cells, a ratio of CD8+ Tₑₘ cells to T_{reg} cells, a ratio of CD4+ Tₑₘ cells to T_{reg} cells, NK cells and B cells in a sample of the subject; and (c) wherein the sample has an increase of the level of one or more biomarkers selected from the group consisting of CD8+ T cells, CD4+ T cells, CD8+ Tₑₘ cells, CD4+ Tₑₘ cells, T_{reg} cells, a ratio of CD8+ Tₑₘ cells to T_{reg} cells, a ratio of CD4+ Tₑₘ cells to T_{reg} cells, NK cells and B cells after administration of the anti-CTLA4 antibody compared to the baseline level of the one or more biomarkers, and/or the sample has a decrease of the level of T_{reg} cells after administration of the anti-CTLA4 antibody compared to the baseline level of the T_{reg} cells, administering to the subject a further cycle of an effective amount of the anti-CTLA4 antibody. In some embodiments, the cancer is a solid cancer, such as advanced-stage and/or metastatic cancer. In some embodiments, the cancer is urothelial carcinoma. In some embodiments, the cancer is resistant or refractory to a prior therapy, wherein the prior therapy is an inhibitor of CTLA4 (e.g., ipilimumab), PD-1 (e.g., pembrolizumab), or a PD-1 ligand. In some embodiments, the one or more biomarkers comprises CD8+ Tₑₘ cells. In some embodiments, the one or more biomarkers comprises CD4+ Tₑₘ cells. In some embodiments, the one or more biomarkers comprises T_{reg} cells. In some embodiments, the one or more biomarkers comprises a ratio of CD8+ Tₑₘ cells to T_{reg} cells. In some embodiments, the one or more biomarkers comprises a ratio of CD4+ Tₑₘ cells to T_{reg} cells. In some embodiments, the one or more biomarkers comprises NK cells. In some embodiments, the antibody is TY21580.

In some embodiments, there is provided a method of providing a prognosis for a subject who has been administered with an effective amount of an anti-CTLA4 antibody, wherein the antibody specifically binds to an epitope comprising amino acid residues Y105 and L106 of human CTLA4 but does not comprise residue I108, wherein the numbering of the amino acid residues is according to SEQ ID NO: 108; the method comprising determining a level of one or more biomarkers selected from the group consisting of CD8+ T cells, CD4+ T cells, CD8+ Tₑₘ cells, CD4+ Tₑₘ cells, T_{reg} cells, a ratio of CD8+ Tₑₘ cells to T_{reg} cells, a ratio of CD4+ Tₑₘ cells to T_{reg} cells, NK cells, B cells in a sample of the subject, wherein: (a) an increase of the level of one or more biomarkers selected from the group consisting of CD8+ T cells, CD4+ T cells, CD8+ Tₑₘ cells, CD4+ Tₑₘ cells, a ratio of CD8+ Tₑₘ cells to T_{reg} cells, a ratio of CD4+ Tₑₘ cells to T_{reg} cells, NK cells and B cells after administration of the anti-CTLA4 antibody compared to the baseline level of the one or more biomarkers indicates an increased likelihood that the subject has an effective response to the CTLA4 antibody; and/or (b) a decrease of the level of T_{reg} cells after administration of the anti-CTLA4 antibody compared to the baseline level of the T_{reg} cells indicates an increased likelihood that the subject has an effective response to the CTLA4 antibody. In some embodiments, the cancer is a solid cancer, such as advanced-stage and/or metastatic cancer. In some embodiments, the cancer is urothelial carcinoma. In some embodiments, the cancer is resistant or refractory to a prior therapy, wherein the prior therapy is an inhibitor of CTLA4 (e.g., ipilimumab), PD-1 (e.g., pembrolizumab), or a PD-1 ligand. In some embodiments, the one or more biomarkers comprises CD8+ Tₑₘ cells. In some embodiments, the one or more biomarkers comprises CD4+ Tₑₘ cells. In some embodiments, the one or more biomarkers comprises T_{reg} cells. In some embodiments, the one or more biomarkers comprises a ratio of CD8+ Tₑₘ cells to T_{reg} cells. In some embodiments, the one or more biomarkers comprises a ratio of CD4+ Tₑₘ cells to T_{reg} cells. In some embodiments, the one or more biomarkers comprises NK cells. In some embodiments, the antibody is TY21580.

In some embodiments, the biomarker is a cell population, such as tumor infiltrating T cells, CD8+ T cells, CD4+ T cells, CD8+ T_{reg} cells, CD4+ T_{reg} cells, NK cells and/or B cells, or a ratio between two cell populations such as a ratio of CD8+ Tₑₘ cells to T_{reg} cells, or a ratio of CD4+ Tₑₘ cells to T_{reg} cells. Suitable methods for determining levels of cell populations in a sample are known in the art, including, for example, fluorescence-activated cell sorting (FACS). In some embodiments, the sample is a blood sample. In some embodiments, the sample is a tumor sample. In some embodiments, the T cells are tumor infiltrating T cells.

In some embodiments, the method comprises determining the level of Tₑₘ cells in a sample. In some embodiments, the Tₑₘ cells are CD8+ Tₑₘ cells. In some embodiments, the Tₑₘ cells are CD4+ Tₑₘ cells. In some embodiments, the Tₑₘ cells are CD45RO+ CCR7− L-selectin-T cells. In some embodiments, the Tₑₘ cells have intermediate to high expression of CD44. In some embodiments, the level of Tₑₘ cells (e.g., CD8+ Tₑₘ cells or CD4+ Tₑₘ cells) increases by at least about 10%, 20%, 30%, 50%, 60%, 70%, 80%, 90%, 100%, 150%, 2 fold, 3 fold, 4 fold, 5 fold or more in a subject likely to respond to the anti-CTLA4 antibody treatment after the subject receives an anti-CTLA4 antibody than the baseline level, e.g., before the subject receives the anti-CTLA4 antibody. In some embodiments, a low level of Tₑₘ cells prior to receiving an anti-CTLA4 antibody in a subject compared to a reference level (e.g., a level of Tₑₘ cells of a healthy subject) indicates that the subject is likely to respond to the anti-CTLA4 antibody treatment. In some embodiments, the low level is no more than any one of 90%, 80%, 70%, 60%, 50%, 40%, 30%, 20%, 10% or less than the reference level.

In some embodiments, the method comprises determining the level of T_{reg} cells in a sample. In some embodiments, the T_{reg} cells are CD4+ CD25+ FOXP3+ T cells. In some embodiments, a decrease in the level of T_{reg} cells after receiving an anti-CTLA4 antibody in a subject compared to a baseline level indicates that the subject is likely to respond to the anti-CTLA4 antibody treatment, e.g., the subject is likely to have stable disease or partial response. In some embodiments, the level of T_{reg} cells decreases by at least about 10%, 20%, 30%, 50%, 60%, 70%, 80%, 90% or more in a subject likely to respond to the anti-CTLA4 antibody treatment after the subject receives an anti-CTLA4 antibody than before the subject receives the anti-CTLA4 antibody.

In some embodiments, the method comprises determining a ratio of the level of Tₑₘ cells to the level of T_{reg} cells, such as a ratio of the level of CD8+ Tₑₘ cells to the level of T_{reg} cells, and/or a ratio of the level of CD8+ Tₑₘ cells to the level of T_{reg} cells after administration of the anti-CTLA4 antibody. In some embodiments, an increase in the ratio of CD8+ Tₑₘ cells to T_{reg} cells after receiving an anti-CTLA4 antibody in a subject compared to a baseline level indicates that the subject is likely to respond to the anti-CTLA4 antibody treatment, e.g., the subject is likely to have stable disease or partial response. In some embodiments, the ratio of CD8+ Tₑₘ cells to T_{reg} cells increases by at least about 10%, 20%, 30%, 50%, 60%, 70%, 80%, 90% or more in a subject likely to respond to the anti-CTLA4 antibody treatment after the subject receives an anti-CTLA4 antibody than before the subject receives the anti-CTLA4 antibody. In some embodiments, an increase in the ratio of CD4+ Tₑₘ cells to T_{reg} cells after receiving an anti-CTLA4 antibody in a subject compared to a baseline level indicates that the subject is likely to respond to the anti-CTLA4 antibody treatment, e.g., the subject is likely to have stable disease or partial response. In some embodiments, the ratio of CD4+ T_{reg} cells to T_{reg} cells increases by at least about 10%, 20%, 30%, 50%, 60%, 70%, 80%, 90% or more in a subject likely to respond to the anti-CTLA4 antibody treatment after the subject receives an anti-CTLA4 antibody than before the subject receives the anti-CTLA4 antibody.

In some embodiments, the method comprises determining the level of NK cells in a sample, such as a blood sample, e.g., peripheral blood sample. In some embodiments, an increase in the level of NK cells after receiving an anti-CTLA4 antibody in a subject compared to the baseline level of NK cells indicates an increased likelihood that the subject has an effective response to the CTLA4 antibody, e.g., the subject is likely to have stable disease or partial response. In some embodiments, the level of NK cells increases by at least about 10%, 20%, 30%, 50%, 60%, 70%, 80%, 90% or more in a subject likely to respond to the anti-CTLA4 antibody treatment after the subject receives an anti-CTLA4 antibody than before the subject receives the anti-CTLA4 antibody. In some embodiments, a low level of NK cells prior to receiving an anti-CTLA4 antibody in a subject compared to a reference level (e.g., a level of NK cells of a healthy subject) indicates that the subject is likely to respond to the anti-CTLA4 antibody treatment. In some embodiments, the low level is no more than any one of 90%, 80%, 70%, 60%, 50%, 40%, 30%, 20%, 10% or less than the reference level.

In some embodiments, the level of a biomarker in a sample is measured by determining the level of RNA transcript expression of the biomarker. Suitable methods of measuring RNA transcript levels in a sample are known in the art, including, for example, by Northern blot analysis, nuclease protection assays, in situ hybridization, PCR analysis (e.g., qPCR, RT-PCR, RT-qPCR, etc.), and next generation sequencing (e.g., RNAseq). In some embodiments, the level of transcript expression of the biomarker is measured by RT-PCR, in situ hybridization, and/or RNAseq.

In some embodiments, the level of a biomarker in a sample is measured by determining the level of protein expression of the biomarker. Suitable methods of measuring protein expression in a sample are known in the art, including, for example, immunoassays (e.g., Meso Scale Discovery or MSD assay), immunohistochemistry (IHC), PET imaging, Western blotting, enzyme-linked immunosorbent assays (ELISAs), flow cytometry, and mass spectrometry. In some embodiments, the level of protein expression of the biomarker is measured by immunoassay, Western blotting, ELISA, IHC, and/or flow cytometry.

In some embodiments, the level of one or more biomarkers is measured in one or more (e.g., one or more, two or more, three or more, four or more, etc.) samples obtained from a subject. Any suitable sample in the form of tissues and/or fluids that are known or believed to contain diseased cells and/or the target of interest may be used in the methods described herein, including, for example, sputum, pleural fluid, lymph fluid, bone marrow, blood, plasma, serum, urine, tissue samples (samples known or expected to contain cancer cells), tumor samples, tumor biopsies, etc. In some embodiments, the sample is a blood sample. In some embodiments, the sample is a serum sample. In some embodiments, the sample is a tumor sample. In some embodiments, the sample is a tumor biopsy. In some embodiments, the sample comprises one or more cancer cells.

Methods of obtaining suitable tissue and/or fluid samples (e.g., methods that are appropriate for obtaining a representative sample from a particular type, location, disease tissue, etc.) are well known to one of ordinary skill in the art, including, for example, by resection, bone marrow biopsy or bone marrow aspiration, endoscopic biopsy or endoscopic aspiration (e.g., cystoscopy, bronchoscopy, colonoscopy, etc.), needle biopsy or needle aspiration (e.g., fine needle aspiration, core needle biopsy, vacuum-assisted biopsy, image-guided biopsy, etc.) skin biopsy (e.g., shave biopsy, punch biopsy, incisional biopsy, excisional biopsy, etc.), various other surgical tissue (e.g., tumor tissue) biopsy and/or excision strategies, and fluid collections (e.g., collecting urine, blood, serum, plasma, sputum, etc.).

In some embodiments, the one or more samples obtained from the subject are enriched for diseased (e.g., cancerous) cells. Methods of enriching a tissue or fluid preparation for diseased (e.g., cancerous) cells are known in the art, including, for example, by separating diseased (e.g., cancerous) cells from normal cells by flow cytometry. In some embodiments, the level of one or more biomarkers is measured in the enriched samples. In some embodiments, the level of one or more biomarkers is measured in samples that have not been enriched or otherwise altered after isolation.

In some embodiments, the one or more samples are fixed (i.e. preserved) by conventional methodology (See e.g., “Manual of Histological Staining Method of the Armed Forces Institute of Pathology,” 3^{rd} edition (1960) Lee G. Luna, HT (ASCP) Editor, The Blakston Division McGraw-Hill Book Company, New York; The Armed Forces Institute of Pathology Advanced Laboratory Methods in Histology and Pathology (1994) Ulreka V. Mikel, Editor, Armed Forces Institute of Pathology, American Registry of Pathology, Washington, D.C.). The choice of a fixative may be determined by one of ordinary skill in the art for the purpose for which the sample is to be analyzed. The length of fixation will depend upon the size and type of the tissue sample and the fixative used (e.g., neutral buffered formalin, paraformaldehyde, etc.), as will be appreciated by one of ordinary skill in the art. In some embodiments, the level of one or more biomarkers is measured in a sample that is fixed. In some embodiments, the level of one or more biomarkers is measured in samples that have not been fixed or otherwise altered after isolation.

In some embodiments, one or more samples are obtained from the subject prior to administration with an anti-CTLA4 antibody. In some embodiments, one or more samples are obtained from the subject after administration of a first and/or subsequent dose of an anti-CTLA4 antibody. In some embodiments, one or more samples are obtained from the subject after completion of an anti-CTLA4 antibody therapy. In some embodiments, one or more samples are obtained from the subject, prior to, during, and after completion of an anti-CTLA4 antibody therapy.

In some embodiments, the method comprises comparing the level of a biomarker in a sample obtained from a subject to a reference level of the biomarker. In some embodiments, the reference level is the level of the biomarker in a reference sample (e.g., a reference cell (such as a cell line, including but not limited to Raji (ATCC, CC-86) or Daudi (ATCC, CCL-213) cell lines), a corresponding sample taken from one or more patients determined to be responsive to anti-CTLA4 antibody therapy, a corresponding sample taken from one or more patients determined to be non-responsive to anti-CTLA4 antibody therapy, a corresponding adjacent normal tissue, etc.). In some embodiments, the reference level is measured in the reference sample using the same method as was used to measure the level of the biomarker in the subject's sample. In some embodiments, the reference level is measured in the reference sample using a different method than was used to measure the level of the biomarker in the subject's sample.

In some embodiments, the reference level is a pre-determined level of a biomarker (e.g., the average level of the biomarker in a database of diseased samples (such as tissue biopsies or serum samples) isolated from multiple reference patients; the average level of the biomarker in a database of samples (such as tissue biopsies or serum samples) isolated from multiple healthy reference patients; etc.).

In some embodiments, the reference level is a baseline level of the biomarker before the subject is administered the anti-CTLA4 antibody.

In some embodiments, the reference level of a biomarker refers to a detectable level of expression. That is to say, in some embodiments, the level of a biomarker measured in the sample obtained from the subject is considered to be lower than a reference level when the level of the biomarker in the sample is undetectable, e.g., below the limit of detection.

In some embodiments, the level of a biomarker measured in the sample obtained from the subject is considered to be lower than the reference level when the level of the biomarker in the sample is at least about 25% lower than the reference level. For example, the level of a biomarker measured in the sample obtained from the subject is considered to be lower than the reference level when the level of the biomarker in the sample is at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% lower than the reference level. In some embodiments, the level of a biomarker measured in the sample obtained from the subject is considered to be lower than the reference level when the level of the biomarker in the sample is at least about 1-fold lower than the reference level. For example, the level of a biomarker measured in the sample obtained from the subject is considered to be lower than the reference level when the level of the biomarker in the sample is at least about 1-fold, at least about 1.5-fold, at least about 2-fold, at least about 2.5-fold, at least about 3-fold, at least about 3.5-fold, at least about 4-fold, at least about 4.5-fold, at least about 5-fold, at least about 5.5-fold, at least about 6-fold, at least about 6.5-fold, at least about 7-fold, at least about 7.5 fold, at least about 8-fold, at least about 8.5-fold, at least about 9-fold, at least about 9.5-fold, at least about 10-fold, at least about 100-fold, or at least about 1000-fold lower than the reference level. In some embodiments, the level of a biomarker in the sample obtained from the subject is below the limit of detection. In some embodiments, the level of a biomarker measured in the sample obtained from the subject is considered to be lower than the reference level when the level of the biomarker in the sample is below the limit of detection while the reference level is above the limit of detection, is detectable, and/or is not zero. In some embodiments, a level is considered to be below the limit of detection when the level does not give an appreciable signal, a detectable signal, and/or is not significantly different than an appropriate negative control when performing an assay for measuring the level of a biomarker (e.g., below the limit of detection of an assay measuring RNA transcript expression of the biomarker (such as RT-PCR, in situ hybridization, and/or next generation sequencing), below the limit of detection of an assay measuring protein expression of a biomarker (such as an immunoassay, PET imaging, Western blotting, ELISA, immunohistochemistry, and/or flow cytometry), etc.).

In some embodiments, a subject is administered an effective amount of an anti-CTLA4 antibody when the level of a biomarker in a sample obtained from the subject is lower than the reference level. In some embodiments, a subject is determined to be likely to respond to an anti-CTLA4 antibody when the level of the biomarker in a sample obtained from the subject is lower than the reference level. In some embodiments, a subject is administered an effective amount of an anti-CTLA4 antibody after the subject has been determined to be likely to respond to the anti-CTLA4 antibody. In some embodiments, a subject having cancer is selected for treatment with an anti-CTLA4 when the level of the biomarker in a sample obtained from the subject is lower than the reference level. In some embodiments, a subject is positively stratified for enrollment into an anti-CTLA4 antibody therapy when the level of a biomarker in a sample obtained from the subject is lower than the reference level.

In some embodiments, the level of a biomarker measured in the sample obtained from the subject is considered to be higher than the reference level when the level of the biomarker in the sample is at least about 5% higher than the reference level. For example, the level of a biomarker measured in the sample obtained from the subject is considered to be higher than the reference level when the level of the biomarker in the sample is at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% higher than the reference level. In some embodiments, the level of a biomarker measured in the sample obtained from the subject is considered to be higher than the reference level when the level of the biomarker in the sample is at least about 1-fold higher than the reference level. For example, the level of a biomarker measured in the sample obtained from the subject is considered to be higher than the reference level when the level of the biomarker in the sample is at least about 1-fold, at least about 1.5-fold, at least about 2-fold, at least about 2.5-fold, at least about 3-fold, at least about 3.5-fold, at least about 4-fold, at least about 4.5-fold, at least about 5-fold, at least about 5.5-fold, at least about 6-fold, at least about 6.5-fold, at least about 7-fold, at least about 7.5 fold, at least about 8-fold, at least about 8.5-fold, at least about 9-fold, at least about 9.5-fold, at least about 10-fold, at least about 100-fold, or at least about 1000-fold higher than the reference level. In some embodiments, the level of a biomarker in the reference sample is below the limit of detection. In some embodiments, the level of a biomarker measured in the sample obtained from the subject is considered to be higher than the reference level when the level of the biomarker in the sample is above the limit of detection, is detectable, and/or is not zero while the level of the biomarker in the reference sample is below the limit of detection. In some embodiments, a level is considered to be below the limit of detection when the level does not give an appreciable signal, a detectable signal, and/or is not significantly different than an appropriate negative control when performing an assay for measuring the level of a biomarker (e.g., below the limit of detection of an assay measuring RNA transcript expression of the biomarker (such as RT-PCR, in situ hybridization, and/or next generation sequencing), below the limit of detection of an assay measuring protein expression of the biomarker (such as an immunoassay, PET imaging, Western blotting, ELISA, immunohistochemistry, and/or flow cytometry), etc.).

In some embodiments, a subject is administered an effective amount of an anti-CTLA4 antibody when the level of a biomarker in a sample obtained from the subject is higher than the reference level. In some embodiments, a subject is determined to be likely to respond to an anti-CTLA4 antibody when the level of a biomarker in a sample obtained from the subject is higher than the reference level. In some embodiments, a subject is administered an effective amount of an anti-CTLA4 antibody after the subject has been determined to be likely to respond to the anti-CTLA4 antibody. In some embodiments, a subject having cancer is selected for treatment with an anti-CTLA4 antibody when the level of expression of a biomarker in a sample obtained from the subject is higher than the reference level. In some embodiments, a subject is positively stratified for enrollment into an anti-CTLA4 antibody therapy when the level of a biomarker in a sample obtained from the subject is higher than the reference level.

In some embodiments, the method comprises determining the level of a biomarker at two or more time points during the course of the anti-CTLA4 antibody treatment. In some embodiments, the method comprises determining the level of a biomarker (e.g., CD8+ Ta cells, CD4+ Tₑₘ cells, a ratio between CD8+ Tₑₘ cells to T_{reg} cells, a ratio between CD4+ Tₑₘ cells to T_{reg} cells, NK cells) in a sample obtained from the subject prior to the administration of the anti-CTLA4 antibody. In some embodiments, the method comprises determining the level of a biomarker in a sample obtained from the subject after the administration of the anti-CTLA4 antibody. In some embodiments, the method comprises determining the level of a biomarker in a sample obtained from the subject after each cycle of anti-CTLA4 antibody treatment. In some embodiments, the method comprises determining a ratio (“induction ratio”) between the difference in the level of the biomarker after administration of the anti-CTLA4 antibody (C2) and the level of the biomarker before administration of the anti-CTLA4 antibody (C1) and the level of the biomarker before administration of the anti-CTLA4 antibody (C1): (C2-C1)/C1. In some embodiments, an induction ratio of at least about any one of 50%, 1, 2, 3, 4, 5, 6, 7, 8, 9, 10 or more indicates a high likelihood of responding to the anti-CTLA4 antibody treatment, i.e., having stable disease (SD) or partial response (PR) after the treatment.

### IV. Anti-CTLA4 Antibodies

The method described herein comprise administration of an anti-CTLA4 antibody that specifically binds to human CTLA4, including CTLA4 antibodies, antigen-binding fragments of the CTLA4 antibodies, and derivatives of the CTLA4 antibodies. Exemplary anti-CTLA4 antibodies have been described, for example, in International Publication No. WO2019149281A1, which is incorporated herein by reference in its entirety.

In some embodiments, the anti-CTLA4 antibody is any one of the antibodies described herein, including antibodies described with reference to specific amino acid sequences of HVRs, variable regions (VL, VH), and light and heavy chains (e.g., IgG1, IgG2, IgG4). In some embodiments, the antibodies are human antibodies. In some embodiments, the antibodies are humanized antibodies and/or chimeric antibodies. In some embodiments, the anti-CTLA4 antibody binds to human CTLA4, and have at least one (e.g., at least one, at least two, at least three, at least four, at least five, at least six, at least seven, at least eight, or all nine) of the following functional properties: (a) bind to human, cynomolgus monkey, mouse, rat, and/or dog CTLA4 with a K_{D} of 500 nM or less; (b) have antagonist activity on human CTLA4; (c) do not bind to human PD-1, PD-L1, PD-L2, LAG3, TIM3, B7-H3, CD95, CD120a, OX40, CD40, BTLA, VISTA, ICOS, and/or B7-H4 at concentration up to 100 nM; (d) are cross-reactive with monkey, mouse, rat, and/or dog CTLA4; (e) induces ADCC effects (e.g., on Tregs); (f) activates human PBMCs (e.g., stimulates secretion of IL-2 and/or IFNγ); (g) are capable of inhibiting tumor cell growth; (h) have therapeutic effect on a cancer; and (i) block binding of human CTLA4 to human CD80 and/or human CD86. In some embodiments, the anti-CTLA4 antibodies described herein have lower activity in blocking binding of CD80 and/or CD86 to human CTLA4 as compared to ipilimumab in an assay wherein either when human CD80 and/or CD86 are immobilized (or plate bound) or when human CTLA4 protein is present on cell surface. In some embodiments, the anti-CTLA4 antibodies described herein deplete T_{reg} cells selectively in tumor microenvironment as compared to T_{reg} depletions in PBMC or spleen. In some embodiments, the anti-CTLA4 antibodies described herein have higher T_{reg} depletion activity in tumor microenvironment as compared to ipilimumab. Also provided herein are one or more anti-CTLA4 antibodies or antigen-binding fragments that cross-compete for binding to human CTLA4 with one or more of the antibodies or antigen-binding fragments described herein.

In some embodiments, the antibodies or antigen-binding fragments bind to human, cynomolgus monkey, mouse, rat, and/or dog CTLA4 with a K_{D} of about 500 nM or less (e.g., about 500 nM or less, about 450 nM or less, about 400 nM or less, about 350 nM or less, about 300 nM or less, about 250 nM or less, about 200 nM or less, about 150 nM or less, about 100 nM or less, about 90 nM or less, about 80 nM or less, about 70 nM or less, about 60 nM or less, about 50 nM or less, about 40 nM or less, about 30 nM or less, about 25 nM or less, about 20 nM or less, about 10 nM or less, about 1 nM or less, about 0.1 nM or less, etc.) In some embodiments, the antibodies or antigen-binding fragments bind to human, cynomolgus monkey, mouse, rat, and/or dog CTLA4 with a K_{D} of about 350 nM or less. In some embodiments, the antibodies or antigen-binding fragments bind to human CTLA4 with a K_{D} of about 100 nM or less. In some embodiments, the antibodies or antigen-binding fragments bind to human CTLA4 with a K_{D} of about 50 nM or less. In some embodiments, the antibodies or antigen-binding fragments bind to human CTLA4 with a K_{D} of about 10 nM or less. Methods of measuring the K_{D} of an antibody or antigen-binding fragment may be carried out using any method known in the art, including for example, by surface plasmon resonance, an ELISA, isothermal titration calorimetry, a filter binding assay, an EMSA, etc. In some embodiments, the K_{D} is measured by surface plasmon resonance or an ELISA (see e.g., Example 3 below).

In some embodiments, the antibodies or antigen-binding fragments described herein have antagonist activity on human CTLA4. In some embodiments, the antibodies or antigen-binding fragments repress one or more activities of human CTLA4 when a cell (e.g., a human cell) expressing human CTLA4 is contacted by the antibody or antigen binding fragment (e.g., CTLA4 blockade as measured by an increase in a reporter gene signal using a CLA4 blockage reporter gene assay).

In some embodiments, the antibodies or antigen-binding fragments are cross-reactive with monkey (e.g., cynomolgus monkey), mouse, rat, and/or dog CTLA4. In some embodiments, the antibodies or antigen-binding fragments are cross-reactive with monkey CTLA4. In some embodiments, the antibodies or antigen-binding fragments are cross-reactive with mouse CTLA4. In some embodiments, the antibodies or antigen-binding fragments are cross-reactive with rat CTLA4. In some embodiments, the antibodies or antigen-binding fragments are cross-reactive with dog CTLA4. In some embodiments, the antibodies or antigen binding fragments are cross reactive with monkey and mouse CTLA4; monkey and rat CTLA4; monkey and dog CTLA4; mouse and rat CTLA4; mouse and dog CTLA4; rat and dog CTLA4; monkey, mouse, and rat CTLA4; monkey, mouse, and dog CTLA4; monkey, rat, and dog CTLA4; mouse, rat, and dog CTLA4; or monkey, mouse, rat, and dog CTLA4. In some embodiments, the antibodies or antigen binding fragments are cross-reactive if the antibodies or antigen-binding fragments binds to a non-human CTLA4 molecule with a K_{D} less than about 500 nM (e.g., less than about 1 nM, less than about 10 nM, less than about 25 nM, less than about 50 nM, less than about 75 nM, less than about 100 nM, less than about 150 nM, less than about 200 nM, less than about 250 nM, less than about 300 nM, less than about 350 nM, etc.). Methods of measuring antibody cross-reactivity are known in the art, including, without limitation, surface plasmon resonance, an ELISA, isothermal titration calorimetry, a filter binding assay, an EMSA, etc. In some embodiments, the cross-reactivity is measured by ELISA.

In some embodiments, the antibodies induce ADCC effects against a CTLA4 expressing cell (e.g., against CTLA4-expressing human cells such as Tregs) after the antibody binds to the cell-expressed CTLA4. Methods of measuring ADCC effects (e.g., in vitro methods) are known in the art. In some embodiments, the antibodies induce ADCC effects by more than about 10% (e.g., induce ADCC by more than about 10%, more than about 15%, more than about 20%, more than about 25%, more than about 30%, more than about 35%, more than about 40%, etc.) relative to a control (e.g., an isotype control or ipilimumab).

In some embodiments, the antibodies or antigen-binding fragments are capable of inhibiting tumor cell growth and/or proliferation. In some embodiments, the tumor cell growth and/or proliferation is inhibited by at least about 5% (e.g., at least about 5%, at least about 10%, at least about 20%, at least about 30%, at least about 40%, at least about 50%, at least about 60%, at least about 70%, at least about 80%, at least about 90%, or at least about 99%) when contacted with the antibodies or antigen-binding fragments relative to corresponding tumor cells not contacted with the antibodies or antigen-binding fragments (or relative to corresponding tumor cells contacted with an isotype control antibody). In some embodiments, the antibodies or antigen-binding fragments are capable of reducing tumor volume in a subject when the subject is administered the antibodies or antigen-binding fragments. In some embodiments, the antibodies or antigen-binding fragments are capable of reducing tumor volume in a subject by at least about 5% (e.g., at least about 5%, at least about 10%, at least about 20%, at least about 30%, at least about 40%, at least about 50%, at least about 60%, at least about 70%, at least about 80%, at least about 90%, or at least about 99%) relative to the initial tumor volume in the subject (e.g., prior to administration of the antibodies or antigen-binding fragments; as compared to a corresponding tumor in a subject administered an isotype control antibody). Methods of monitoring tumor cell growth/proliferation, tumor volume, and/or tumor inhibition are known in the art.

In some embodiments, the antibodies or antigen-binding fragments have therapeutic effect on a cancer. In some embodiments, the antibodies or antigen-binding fragments reduce one or more signs or symptoms of a cancer. In some embodiments, a subject suffering from a cancer goes into partial or complete remission when administered the antibodies or antigen-binding fragments.

In another aspect, the disclosure provides isolated antibodies that compete or cross-compete for binding to human CTLA4 with any of the illustrative antibodies of the disclosure, such as TY21585, TY21586, TY21587, TY21588, TY21589, TY21580, TY21591, TY21686, TY21687, TY21689, TY21680, TY21691, and/or TY21692. In a particular embodiment, the present application provides isolated antibodies that compete or cross-compete for binding to the same epitope on the human CTLA4 with any of the illustrative antibodies of the disclosure. The ability of an antibody to compete or cross-compete for binding with another antibody can be determined using standard binding assays known in the art, such as BIAcore analysis, ELISA assays, or flow cytometry. For example, one can allow an illustrative antibody of the disclosure to bind to human CTLA4 under saturating conditions and then measure the ability of the test antibody to bind to the CTLA4. If the test antibody is able to bind to the CTLA4 at the same time as the illustrative antibody, then the test antibody binds to a different epitope as the illustrative antibody. However, if the test antibody is not able to bind to the CTLA4 at the same time, then the test antibody binds to the same epitope, an overlapping epitope, or an epitope that is in close proximity to the epitope bound by the illustrative antibody. This experiment can be performed using various methods, such as ELISA, RIA, FACS or surface plasmon resonance.

In some embodiments, the antibodies or antigen-binding fragments block the binding between CTLA4 and one or more of its binding partners (e.g., human CTLA4 and human CD80, human CTLA4 and human CD86). In some embodiments, the antibodies or antigen-binding fragments block the binding between CTLA4 and its ligand in vitro. In some embodiments, the antibody or antigen-binding fragment has a half maximal inhibitory concentration (IC₅₀) of about 500 nM or less (e.g., about 500 nM or less, about 400 nM or less, about 300 nM or less, about 200 nM or less, about 100 nM or less, about 50 nM or less, about 25 nM or less, about 10 nM or less, about 1 nM or less, etc.) for blocking binding of CTLA4 to CD80 and/or CD86. In some embodiments, the antibody or antigen-binding fragment has a half maximal inhibitory concentration (IC₅₀) of about 100 nM or less for blocking binding of CTLA4 to CD80 and/or CD86. In some embodiments, the antibody or antigen-binding fragment completely blocks binding of human CTLA4 to CD80 and/or CD86 when provided at a concentration of about 100 nM or greater (e.g., about 100 nM or greater, about 500 nM or greater, about 1 μM or greater, about 10 μM or greater, etc.). As used herein, the term “complete blocking” or “completely blocks” refers to the antibody or antigen-binding fragment's ability to reduce binding between a first protein and a second protein by at least about 80% (e.g., at least about 80%, at least about 85%, at least about 90%, at least about 95%, at least about 99%, etc.). Methods of measuring the ability of an antibody or antigen-binding fragment to block binding of a first protein (e.g., human CTLA4) and a second protein (e.g., human CD80 or human CD86) are known in the art, including, without limitation, via BIAcore analysis, ELISA assays, and flow cytometry. In some embodiments, the anti-CTLA4 antibodies described herein have lower activity in blocking ligand binding than ipilimumab.

In some embodiments, the anti-CTLA4 antibody binds human CTLA4 with a K_{D} of 1000 nM or less (e.g., 50 nM or less, 10 nM or less) as measured by surface plasmon resonance. In some embodiments, the antibody is cross-reactive with at least one non-human species selected from cynomolgus monkey, mouse, rat, and dog.

In some embodiments, the anti-CTLA4 antibody specifically binds to an epitope similar to a ligand binding site of human CTLA4. In some embodiments, the antibody specifically binds to an epitope similar to CD80 binding site of human CTLA4. In some embodiments, the antibody specifically binds to an epitope similar to CD86 binding site of human CTLA4. In some embodiments, the antibody specifically binds to an epitope comprising one or more amino acid residues in a ligand binding site (e.g., CD80 and/or CD86 binding site) of human CTLA4. In some embodiments, the antibody specifically binds to an epitope on human CTLA4 that is different from the epitope of ipilimumab. In some embodiments, the epitope does not comprise amino acid residues in the CC′ loop motif of human CTLA4. In some embodiments, the epitope does not comprise amino acid residue L106 or I108 of human CTLA4. In some embodiments, the antibody specifically binds to an epitope comprising amino acid residues Y105 and L106, but not I108 of human CTLA4, wherein the numbering of the amino acid residues is according to

(SEQ ID NO: 108)
KAMHVAQPAVVLASSRGIASFVCEYASPGKATEVRVTVLRQADSQVT
EVCAATYMMGNELTFLDDSICTGTSSGNQVNLTIQGLRAMDTGLYIC
KVELMYPPPYYLGIGNGTQIYVIDPE

In some embodiments, the anti-CTLA4 antibody comprising a heavy chain variable region and a light chain variable region, a) where the heavy chain variable region comprises an HVR-H1, an HVR-H2, and an HVR-H3, where the HVR-H1 comprises an amino acid sequence according to a formula selected from: Formula (I): X1TFSX2YX3IHWV (SEQ ID NO: 1), where X1 is F or Y, X2 is D or G, and X3 is A, G, or W; Formula (II): YSIX1SGX2X3WX4WI (SEQ ID NO: 2), where X1 is S or T, X2 is H or Y, X3 is H or Y, and X4 is A, D, or S; and Formula (III): FSLSTGGVAVX1WI (SEQ ID NO: 3), where X1 is G or S; the HVR-H2 comprises an amino acid sequence according to a formula selected from: Formula (IV): IGX1IX2HSGSTYYSX3SLKSRV (SEQ ID NO: 4), where X1 is D or E, X2 is S or Y, and X3 is P or Q; Formula (V): IGX1ISPSX2GX3TX4YAQKFQGRV (SEQ ID NO: 5), where X1 is I or W, X2 is G or S, X3 is G or S, and X4 is K or N; and Formula (VI): VSX1ISGX2GX3X4TYYADSVKGRF (SEQ ID NO: 6), where X1 is A, G, or S, X2 is S or Y, X3 is G or S, and X4 is S or T; and the HVR-H3 comprises an amino acid sequence according to a formula selected from: Formula (VII): ARX1X2X3X4FDX5 (SEQ ID NO: 7), where X1 is G, R, or S, X2 is A, 1, or Y, X3 is D, V, or Y, X4 is A, E, or Y, and X5 is I or Y; Formula (VIII): ARX1GX2GYFDX3 (SEQ ID NO: 8), where X1 is D or L, X2 is F or Y, and X3 is V or Y; Formula (IX): ARX1X2X3X4AX5X6FDY (SEQ ID NO: 9), where X1 is L or R, X2 is I or P, X3 is A or Y, X4 is S or T, X5 is T or Y, and X6 is A or Y; and Formula (X): ARDX1X2X3GSSGYYX4GFDX5 (SEQ ID NO: 10), where X1 is I or V, X2 is A or H, X3 is P or S, X4 is D or Y, and X5 is F or V; and/or b) where the light chain variable region comprises an HVR-L1, an HVR-L2, and an HVR-L3, where the HVR-L1 comprises an amino acid sequence according to a formula selected from: Formula (XI): RASQX1X2X3SX4LX5 (SEQ ID NO: 11), where X1 is G or S, X2 is I or V, X3 is G or S, X4 is S or Y, and X5 is A or N; Formula (XII): RASQX1VX2X3RX4LA (SEQ ID NO: 12), where X1 is S or T, X2 is F, R, or S, X3 is G or S, and X4 is F or Y; and Formula (XIII): RASX1SVDFX2GX3SFLX4 (SEQ ID NO: 13), where X1 is E or Q, X2 is D, F, H, or Y, X3 is F, I, or K, and X4 is A, D, or H; the HVR-L2 comprises an amino acid sequence according to Formula (XIV): X1ASX2X3X4X5GX6 (SEQ ID NO: 14), where X1 is A or D, X2 is N, S, or T, X3 is L or R, X4 is A, E, or Q, X5 is S or T, and X6 is I or V; and the HVR-L3 comprises an amino acid sequence according to a formula selected from: Formula (XV): YCX1X2X3X4X5X6PX7T (SEQ ID NO: 15), where X1 is E, Q, or V, X2 is H or Q, X3 is A, G, H, R, or S, X4 is D, L, S, or Y, X5 is E, G, P, Q, or S, X6 is L, T, V, or W, and X7 is F, L, P, W, or Y; Formula (XVI): YCQQX1X2X3WPPWT (SEQ ID NO: 16), where X1 is S or Y, X2 is D or Y, and X3 is Q or Y; and Formula (XVII): YCQX1YX2SSPPX3YT (SEQ ID NO: 17), where X1 is H or Q, X2 is T or V, and X3 is E or V.

In some embodiments, the antibody comprises: a) an HVR-H1 comprising an amino acid sequence selected from SEQ ID NOS: 18-29; an HVR-H2 comprising an amino acid sequence selected from SEQ ID NOS. 30-39; and an HVR-H3 comprising an amino acid sequence selected from SEQ ID NOS: 40-52; and/or b) an HVR-L1 comprising an amino acid sequence selected from SEQ ID NOS: 53-65; an HVR-L2 comprising an amino acid sequence selected from SEQ ID NOS: 66-69 and an HVR-L3 comprising an amino acid sequence selected from SEQ ID NOS: 70-81. In some embodiments, the antibody comprises one, two, three, four, five, or all six of the HVRs shown for any of the exemplary antibodies described in Table A below.

TABLE A
anti-CTLA4 HVR sequences

| Ab | | | | | | |
|---|---|---|---|---|---|---|
| name: | HVR-H1 | HVR-H2 | HVR-H3 | HVR-L1 | HVR-L2 | HVR-L3 |
| TY21585 | FTFSDYAI | IGIISPSSGSTNYA | ARDIHSGSSGY | RASESVDFF | DASNR | YCQHYTSS |
| | HWV | QKFQGRV | YYGFDV | GISFLA | ATGI | PPVYT |
| | (SEQ ID | (SEQ ID NO: 30) | (SEQ ID NO: 40) | (SEQ ID NO: | (SEQ ID | (SEQ ID |
| | NO: 18) | | | 53) | NO: 66) | NO: 70) |
| TY21586 | YSITSGYY | VSSISGSGSTTYYA | ARDGFGYFDY | SASSSVSYV | DASSLE | YCVQGLQ |
| | WAWI | DSVKGRF | (SEQ ID NO: 41) | Y | SGV | TPWT |
| | (SEQ ID | (SEQ ID NO: 31) | | (SEQ ID NO: | (SEQ ID | (SEQ ID |
| | NO: 19) | | | 54) | NO: 67) | NO: 71) |
| TY21587 | FTFSDYGI | IGEIYHSGSTYYSP | ARDVAPGSSG | RASQGIGSSL | DASNR | YCQQYDQ |
| | HWV | SLKSRV | YYDGFDF | A | ATGI | WPPWT |
| | (SEQ ID | (SEQ ID NO: 32) | (SEQ ID NO: 42) | (SEQ ID NO: | (SEQ ID | (SEQ ID |
| | NO: 20) | | | 55) | NO: 66) | NO: 72) |
| TY21588 | YSISSGYH | VSGISGYGGSTYY | ARHSYYGSGNFDY | RASESVDFF | DASNL | YCQQSYS |
| | WDWI | ADSVKGRF | (SEQ ID NO: 43) | GKSFLH | ETGV | WPPT |
| | (SEQ ID | (SEQ ID NO: 33) | | (SEQ ID NO: | (SEQ ID | (SEQ ID |
| | NO: 21) | | | 56) | NO: 68) | NO: 73) |
| TY21589 | FTFSDYWI | IGWISPSGGGTKY | ARGAYEFDY | RASQSVSSR | DASNR | YCQQSYPT |
| | HWV | AQKFQGRV | (SEQ ID NO: 44) | FLA | ATGI | PLT |
| | (SEQ ID | (SEQ ID NO: 34) | | (SEQ ID NO: | (SEQ ID | (SEQ ID |
| | NO: 22) | | | 57) | NO: 66) | NO: 74) |
| TY21580 | YSISSGYH | LARIDWDDDKYY | ARSYVYFDY | RASQSVRGR | DASNR | YCQQSSSW |
| | WSWI | STSLKSRL | (SEQ ID NO: 45) | FLA | ATGI | PPT |
| | (SEQ ID | (SEQ ID NO: 35) | | (SEQ ID NO: | (SEQ ID | (SEQ ID |
| | NO: 23) | | | 58) | NO: 66) | NO: 75) |
| TY21591 | FSLSTGGV | IGEIYHSGSTYYSP | ARRIATATYFDY | RASQTVFSR | DASNR | YCQQSYY |
| | AVSWI | SLKSRV | (SEQ ID NO: 46) | YLA | ATGI | WPPWT |
| | (SEQ ID | (SEQ ID NO: 32) | | (SEQ ID NO: | (SEQ ID | (SEQ ID |
| | NO: 24) | | | 59) | NO: 66) | NO: 76) |
| TY21686 | FSLSTGGV | VSAISGYGSTTYY | ARLPYSAYAF | RASQGVSSY | AASTL | YCQHHYG |
| | AVGWI | ADSVKGRF | DY | LA | QSGV | TPLT |
| | (SEQ ID | (SEQ ID NO: 36) | (SEQ ID NO: 47) | (SEQ ID NO: | (SEQ ID | (SEQ ID |
| | NO: 25) | | | 60) | NO: 69) | NO: 77) |
| TY21687 | FTFSGYAI | IGIISPSGGGTKYA | ARHPFAY | RASQSVDFY | DASNR | YCQQYVSS |
| | HWV | QKFQGRV | (SEQ ID NO: 48) | GISFLD | ATGI | PPEYT |
| | (SEQ ID | (SEQ ID NO: 37) | | (SEQ ID NO: | (SEQ ID | (SEQ ID |
| | NO: 26) | | | 61) | NO: 66) | NO: 78) |
| TY21689 | YTFSGYGI | IGEIYHSGSTYYSP | ARRIDAFDI | RASQSVDFD | DASSLE | YCQQRDS |
| | HWV | SLKSRV | (SEQ ID NO: 49) | GFSFLH | SGV | WPYT |
| | (SEQ ID | (SEQ ID NO: 32) | | (SEQ ID NO: | (SEQ ID | (SEQ ID |
| | NO: 27) | | | 62) | NO: 67) | NO: 79) |
| TY21680 | YTFSGYAI | IGIISPSGGGTKYA | ARLYDVAY | RASQSVDFH | DASSLE | YCEQSLEV |
| | HWV | QKFQGRV | (SEQ ID NO: 50) | GKSFLH | SGV | PFT |
| | (SEQ ID | (SEQ ID NO: 37) | | (SEQ ID NO: | (SEQ ID | (SEQ ID |
| | NO: 28) | | | 63) | NO: 67) | NO: 80) |
| TY21691 | FTFSDYAI | IGIISPSGGSTKYA | ARLGYGYFDV | RASQSVDFY | DASSLE | YCVQALQ |
| | HWV | QKFQGRV | (SEQ ID NO: 51) | GISFLH | SGV | LPLT |
| | (SEQ ID | (SEQ ID NO: 38) | | (SEQ ID NO: | (SEQ ID | (SEQ ID |
| | NO: 18) | | | 64) | NO: 67) | NO: 81) |
| TY21692 | YSITSGHY | IGDISHSGSTYYSQ | ARGSRTGYFDY | RASQSISSYL | DASNL | YCQHHYG |
| | WSWI | SLKSRV | (SEQ ID NO: 52) | N | ETGV | TPLT |
| | (SEQ ID | (SEQ ID NO: 39) | | (SEQ ID NO: | (SEQ ID | (SEQ ID |
| | NO: 29) | | | 65) | NO: 68) | NO: 77) |

In some embodiments, the antibody comprises an HVR-H1 comprising the amino acid sequence of SEQ ID NO. 18, an HVR-H2 comprising the amino acid sequence of SEQ ID NO: 30, an HVR-H3 comprising the amino acid sequence of SEQ ID NO: 40, an HVR-L1 comprising the amino acid sequence of SEQ ID NO: 53, an HVR-L2 comprising the amino acid sequence of SEQ ID NO: 66, and an HVR-L3 comprising the amino acid sequence of SEQ ID NO: 70. In some embodiments, the antibody comprises an HVR-H1 comprising the amino acid sequence of SEQ ID NO: 19, an HVR-H2 comprising the amino acid sequence of SEQ ID NO: 31, an HVR-H3 comprising the amino acid sequence of SEQ ID NO: 41, an HVR-L1 comprising the amino acid sequence of SEQ ID NO: 54, an HVR-L2 comprising the amino acid sequence of SEQ ID NO: 67, and an HVR-L3 comprising the amino acid sequence of SEQ ID NO: 71. In some embodiments, the antibody comprises an HVR-H1 comprising the amino acid sequence of SEQ ID NO. 20, an HVR-H2 comprising the amino acid sequence of SEQ ID NO: 32, an HVR-H3 comprising the amino acid sequence of SEQ ID NO: 42, an HVR-L1I comprising the amino acid sequence of SEQ ID NO: 55, an HVR-L2 comprising the amino acid sequence of SEQ ID NO: 66, and an HVR-L3 comprising the amino acid sequence of SEQ ID NO: 72. In some embodiments, the antibody comprises an HVR-H1 comprising the amino acid sequence of SEQ ID NO: 21 an HVR-H2 comprising the amino acid sequence of SEQ ID NO: 33, an HVR-H3 comprising the amino acid sequence of SEQ ID NO: 43, an HVR-L1 comprising the amino acid sequence of SEQ ID NO: 56, an HVR-L2 comprising the amino acid sequence of SEQ ID NO: 68, and an HVR-L3 comprising the amino acid sequence of SEQ ID NO: 73. In some embodiments, the antibody comprises an HVR-H1 comprising the amino acid sequence of SEQ ID NO: 22, an HVR-H2 comprising the amino acid sequence of SEQ ID NO: 34, an HVR-H3 comprising the amino acid sequence of SEQ ID NO: 44, an HVR-L1 comprising the amino acid sequence of SEQ ID NO: 57, an HVR-L2 comprising the amino acid sequence of SEQ ID NO: 66, and an HVR-L3 comprising the amino acid sequence of SEQ ID NO: 74. In some embodiments, the antibody comprises an HVR-H1 comprising the amino acid sequence of SEQ ID NO: 23, an HVR-H2 comprising the amino acid sequence of SEQ ID NO: 35, an HVR-H3 comprising the amino acid sequence of SEQ ID NO: 45, an HVR-L1 comprising the amino acid sequence of SEQ ID NO: 58, an HVR-L2 comprising the amino acid sequence of SEQ ID NO: 66, and an HVR-L3 comprising the amino acid sequence of SEQ ID NO: 75. In some embodiments, the antibody comprises an HVR-H1 comprising the amino acid sequence of SEQ ID NO: 24, an HVR-H2 comprising the amino acid sequence of SEQ ID NO: 32, an HVR-H3 comprising the amino acid sequence of SEQ ID NO: 46, an HVR-L1 comprising the amino acid sequence of SEQ ID NO: 59, an HVR-L2 comprising the amino acid sequence of SEQ ID NO: 66, and an HVR-L3 comprising the amino acid sequence of SEQ ID NO: 76. In some embodiments, the antibody comprises an HVR-H1 comprising the amino acid sequence of SEQ ID NO: 25, an HVR-H2 comprising the amino acid sequence of SEQ ID NO: 36, an HVR-H3 comprising the amino acid sequence of SEQ ID NO: 47, an HVR-L1 comprising the amino acid sequence of SEQ ID NO: 60, an HVR-L2 comprising the amino acid sequence of SEQ ID NO: 69, and an HVR-L3 comprising the amino acid sequence of SEQ ID NO: 77. In some embodiments, the antibody comprises an HVR-H1 comprising the amino acid sequence of SEQ ID NO: 26, an HVR-H2 comprising the amino acid sequence of SEQ ID NO: 37, an HVR-H3 comprising the amino acid sequence of SEQ ID NO: 48, an HVR-L1 comprising the amino acid sequence of SEQ ID NO: 61, an HVR-L2 comprising the amino acid sequence of SEQ ID NO: 66, and an HVR-L3 comprising the amino acid sequence of SEQ ID NO: 78. In some embodiments, the antibody comprises an HVR-H1 comprising the amino acid sequence of SEQ ID NO. 27, an HVR-H2 comprising the amino acid sequence of SEQ ID NO: 32, an HVR-H3 comprising the amino acid sequence of SEQ ID NO: 49, an HVR-L1 comprising the amino acid sequence of SEQ ID NO: 62, an HVR-L2 comprising the amino acid sequence of SEQ ID NO: 67, and an HVR-L3 comprising the amino acid sequence of SEQ ID NO: 79. In some embodiments, the antibody comprises an HVR-H1 comprising the amino acid sequence of SEQ ID NO: 28, an HVR-H2 comprising the amino acid sequence of SEQ ID NO: 37, an HVR-H3 comprising the amino acid sequence of SEQ ID NO: 50, an HVR-L1 comprising the amino acid sequence of SEQ ID NO: 63, an HVR-L2 comprising the amino acid sequence of SEQ ID NO: 67, and an HVR-L3 comprising the amino acid sequence of SEQ ID NO: 80. In some embodiments, the antibody comprises an HVR-H1 comprising the amino acid sequence of SEQ ID NO: 18, an HVR-H2 comprising the amino acid sequence of SEQ ID NO: 38, an HVR-H3 comprising the amino acid sequence of SEQ ID NO: 51, an HVR-L1 comprising the amino acid sequence of SEQ ID NO: 64, an HVR-L2 comprising the amino acid sequence of SEQ ID NO: 67, and an HVR-L3 comprising the amino acid sequence of SEQ ID NO: 81. In some embodiments, the antibody comprises an HVR-H1 comprising the amino acid sequence of SEQ ID NO: 29, an HVR-H2 comprising the amino acid sequence of SEQ ID NO: 39, an HVR-H3 comprising the amino acid sequence of SEQ ID NO: 52, an HVR-L1 comprising the amino acid sequence of SEQ ID NO: 65, an HVR-L2 comprising the amino acid sequence of SEQ ID NO: 68, and an HVR-L3 comprising the amino acid sequence of SEQ ID NO: 77.

In some embodiments, the antibody comprises: a) a heavy chain variable region comprising an amino acid sequence selected from SEQ ID NOS: 82-94, and/or b) a light chain variable region comprising an amino acid sequence selected from SEQ ID NOS: 95-107. In some embodiments, the antibody comprises a heavy chain variable region comprising an amino acid sequence having at least 90% (e.g., at least 90%, 91%, 92%, 93%, 94%, 95%, 96%, 97%, 98%, or 99%) sequence identity to a sequence selected from SEQ ID NOS: 82-94, and/or a light chain variable region comprising an amino acid sequence having at least 90% (e.g., at least 90%, 91%, 92%, 93%, 94%, 95%, 96%, 97%, 98%, or 99%) sequence identity to a sequence selected from SEQ ID NOS: 95-107. In some embodiments, the antibody comprises a heavy chain variable region and a light chain variable region of any of the exemplary antibodies described in Table B below. In some embodiments, the antibody comprises one, two, or all three HVRs of the heavy chain variable region, and/or one, two, or all three HVRs of the light chain variable region shown for any of the exemplary antibodies described in Table B below.

TABLE B
anti-CTLA4 variable region amino acid sequences

| Ab | | |
|---|---|---|
| name: | VH: | VL: |
| TY21585 | EVQLVESGGGLVQPGGSLRLSCAASGFTFSDY | DIQLTQSPSSLSASVGDRVTITCRASESVDFF |
| | AIHWVRQAPGKGLEWIGIISPSSGSTNYAQKF | GISFLAWYQQKPGKAPKLLIYDASNRATGI |
| | QGRVTISRDNSKNTLYLQLNSLRAEDTAVYYC | PSRFSGSGSGTDFTLTISSLQPEDFATYYCQ |
| | ARDIHSGSSGYYYGFDVWGQGTLVTVSS | HYTSSPPVYTFGQGTKVEIKR |
| | (SEQ ID NO: 82) | (SEQ ID NO: 95) |
| TY21586 | EVQLVESGGGLVQPGGSLRLSCAASGYSITSG | DIQLTQSPSSLSASVGDRVTITCSASSSVSYV |
| | YYWAWIRQAPGKGLEWVSSISGSGSTTYYAD | YWYQQKPGKAPKLLIYDASSLESGVPSRFS |
| | SVKGRFTISRDNSKNTLYLQLNSLRAEDTAVY | GSGSGTDFTLTISSLQPEDFATYYCVQGLQT |
| | YCARDGFGYFDYWGQGTLVTVSS | PWTFGQGTKVEIKR |
| | (SEQ ID NO: 83) | (SEQ ID NO: 96) |
| TY21587 | EVQLVESGGGLVQPGGSLRLSCAASGFTFSDY | DIQLTQSPSSLSASVGDRVTITCRASQGIGSS |
| | GIHWVRQAPGKGLEWIGEIYHSGSTYYSPSLK | LAWYQQKPGKAPKLLIYDASNRATGIPSRF |
| | SRVTISRDNSKNTLYLQLNSLRAEDTAVYYCA | SGSGSGTDFTLTISSLQPEDFATYYCQQYDQ |
| | RDVAPGSSGYYDGFDFWGQGTLVTVSS | WPPWTFGQGTKVEIKR |
| | (SEQ ID NO: 84) | (SEQ ID NO: 97) |
| TY21588 | EVQLVESGGGLVQPGGSLRLSCAASGYSISSG | DIQLTQSPSSLSASVGDRVTITCRASESVDFF |
| | YHWDWIRQAPGKGLEWVSGISGYGGSTYYA | GKSFLHWYQQKPGKAPKLLIYDASNLETG |
| | DSVKGRFTISRDNSKNTLYLQLNSLRAEDTAV | VPSRFSGSGSGTDFTLTISSLQPEDFATYYC |
| | YYCARHSYYGSGNFDYWGQGTLVTVSS | QQSYSWPPTFGQGTKVEIKR |
| | (SEQ ID NO: 85) | (SEQ ID NO: 98) |
| TY21589 | EVQLVESGGGLVQPGGSLRLSCAASGFTFSDY | DIQLTQSPSSLSASVGDRVTITCRASQSVSS |
| | WIHWVRQAPGKGLEWIGWISPSGGGTKYAQK | RFLAWYQQKPGKAPKLLIYDASNRATGIPS |
| | FQGRVTISRDNSKNTLYLQLNSLRAEDTAVYY | RFSGSGSGTDFTLTISSLQPEDFATYYCQQS |
| | CARGAYEFDYWGQGTLVTVSS | YPTPLTFGQGTKVEIKR |
| | (SEQ ID NO: 86) | (SEQ ID NO: 99) |
| TY21580 | EVQLVESGGGLVQPGGSLRLSCAASGYSISSG | DIQLTQSPSSLSASVGDRVTITCRASQSVRG |
| | YHWSWIRQAPGKGLEWLARIDWDDDKYYST | RFLAWYQQKPGKAPKLLIYDASNRATGIPS |
| | SLKSRLTISRDNSKNTLYLQLNSLRAEDTAVYY | RFSGSGSGTDFTLTISSLQPEDFATYYCQQS |
| | CARSYVYFDYWGQGTLVTVSS | SSWPPTFGQGTKVEIKR |
| | (SEQ ID NO: 87) | (SEQ ID NO: 100) |
| TY21591 | EVQLVESGGGLVQPGGSLRLSCAASGFSLSTG | DIQLTQSPSSLSASVGDRVTITCRASQTVFS |
| | GVAVSWIRQAPGKGLEWIGEIYHSGSTYYSPS | RYLAWYQQKPGKAPKLLIYDASNRATGIPS |
| | LKSRVTISRDNSKNTLYLQLNSLRAEDTAVYY | RFSGSGSGTDFTLTISSLQPEDFATYYCQQS |
| | CARRIATATYFDYWGQGTLVTVSS | YYWPPWTFGQGTKVEIKR |
| | (SEQ ID NO: 88) | (SEQ ID NO: 101) |
| TY21686 | EVQLVESGGGLVQPGGSLRLSCAASGFSLSTG | DIQLTQSPSSLSASVGDRVTITCRASQGVSS |
| | GVAVGWIRQAPGKGLEWVSAISGYGSTTYYA | YLAWYQQKPGKAPKLLIYAASTLQSGVPSR |
| | DSVKGRFTISRDNSKNTLYLQLNSLRAEDTAV | FSGSGSGTDFTLTISSLQPEDFATYYCQHHY |
| | YYCARLPYSAYAFDYWGQGTLVTVSS | GTPLTFGQGTKVEIKR |
| | (SEQ ID NO: 89) | (SEQ ID NO: 102) |
| TY21687 | EVQLVESGGGLVQPGGSLRLSCAASGFTFSGY | DIQLTQSPSSLSASVGDRVTITCRASQSVDE |
| | AIHWVRQAPGKGLEWIGIISPSGGGTKYAQKF | YGISFLDWYQQKPGKAPKLLIYDASNRATG |
| | QGRVTISRDNSKNTLYLQLNSLRAEDTAVYYC | IPSRFSGSGSGTDFTLTISSLQPEDFATYYCQ |
| | ARHPFAYWGQGTLVTVSS | QYVSSPPEYTFGQGTKVEIKR |
| | (SEQ ID NO: 90) | (SEQ ID NO: 103) |
| TY21689 | EVQLVESGGGLVQPGGSLRLSCAASGYTFSGY | DIQLTQSPSSLSASVGDRVTITCRASQSVDF |
| | GIHWVRQAPGKGLEWIGEIYHSGSTYYSPSLK | DGFSFLHWYQQKPGKAPKLLIYDASSLESG |
| | SRVTISRDNSKNTLYLQLNSLRAEDTAVYYCA | VPSRFSGSGSGTDFTLTISSLQPEDFATYYC |
| | RRIDAFDIWGQGTLVTVSS | QQRDSWPYTFGQGTKVEIKR |
| | (SEQ ID NO: 91) | (SEQ ID NO: 104) |
| TY21680 | EVQLVESGGGLVQPGGSLRLSCAASGYTFSGY | DIQLTQSPSSLSASVGDRVTITCRASQSVDF |
| | AIHWVRQAPGKGLEWIGIISPSGGGTKYAQKF | HGKSFLHWYQQKPGKAPKLLIYDASSLES |
| | QGRVTISRDNSKNTLYLQLNSLRAEDTAVYYC | GVPSRFSGSGSGTDFTLTISSLQPEDFATYY |
| | ARLYDVAYWGQGTLVTVSS | CEQSLEVPFTFGQGTKVEIKR |
| | (SEQ ID NO: 92) | (SEQ ID NO: 105) |
| TY21691 | EVQLVESGGGLVQPGGSLRLSCAASGFTFSDY | DIQLTQSPSSLSASVGDRVTITCRASQSVDF |
| | AIHWVRQAPGKGLEWIGIISPSGGSTKYAQKF | YGISFLHWYQQKPGKAPKLLIYDASSLESG |
| | QGRVTISRDNSKNTLYLQLNSLRAEDTAVYYC | VPSRFSGSGSGTDFTLTISSLQPEDFATYYC |
| | ARLGYGYFDVWGQGTLVTVSS | VQALQLPLTFGQGTKVEIKR |
| | (SEQ ID NO: 93) | (SEQ ID NO: 106) |
| TY21692 | EVQLVESGGGLVQPGGSLRLSCAASGYSITSG | DIQLTQSPSSLSASVGDRVTITCRASQSISSY |
| | HYWSWIRQAPGKGLEWIGDISHSGSTYYSQSL | LNWYQQKPGKAPKLLIYDASNLETGVPSR |
| | KSRVTISRDNSKNTLYLQLNSLRAEDTAVYYC | FSGSGSGTDFTLTISSLQPEDFATYYCQHHY |
| | ARGSRTGYFDYWGQGTLVTVSS | GTPLTFGQGTKVEIKR |
| | (SEQ ID NO: 94) | (SEQ ID NO: 107) |

In some embodiments, the antibody comprises a heavy chain variable region comprising the amino acid sequence of SEQ ID NO: 82, and a light chain variable region comprising the amino acid sequence of SEQ ID NO: 95. In some embodiments, the antibody comprises a heavy chain variable region comprising the amino acid sequence of SEQ ID NO: 83, and a light chain variable region comprising the amino acid sequence of SEQ ID NO: 96. In some embodiments, the antibody comprises a heavy chain variable region comprising the amino acid sequence of SEQ ID NO: 84, and a light chain variable region comprising the amino acid sequence of SEQ ID NO: 97. In some embodiments, the antibody comprises a heavy chain variable region comprising the amino acid sequence of SEQ ID NO: 85, and a light chain variable region comprising the amino acid sequence of SEQ ID NO: 98. In some embodiments, the antibody comprises a heavy chain variable region comprising the amino acid sequence of SEQ ID NO: 86, and a light chain variable region comprising the amino acid sequence of SEQ ID NO: 99. In some embodiments, the antibody comprises a heavy chain variable region comprising the amino acid sequence of SEQ ID NO: 87, and a light chain variable region comprising the amino acid sequence of SEQ ID NO: 100. In some embodiments, the antibody comprises a heavy chain variable region comprising the amino acid sequence of SEQ ID NO: 88, and a light chain variable region comprising the amino acid sequence of SEQ ID NO: 101. In some embodiments, the antibody comprises a heavy chain variable region comprising the amino acid sequence of SEQ ID NO: 89, and a light chain variable region comprising the amino acid sequence of SEQ ID NO: 102. In some embodiments, the antibody comprises a heavy chain variable region comprising the amino acid sequence of SEQ ID NO: 90, and a light chain variable region comprising the amino acid sequence of SEQ ID NO: 103. In some embodiments, the antibody comprises a heavy chain variable region comprising the amino acid sequence of SEQ ID NO: 91, and a light chain variable region comprising the amino acid sequence of SEQ ID NO: 104. In some embodiments, the antibody comprises a heavy chain variable region comprising the amino acid sequence of SEQ ID NO: 92, and a light chain variable region comprising the amino acid sequence of SEQ ID NO: 105. In some embodiments, the antibody comprises a heavy chain variable region comprising the amino acid sequence of SEQ ID NO: 93, and a light chain variable region comprising the amino acid sequence of SEQ ID NO: 106. In some embodiments, the antibody comprises a heavy chain variable region comprising the amino acid sequence of SEQ ID NO: 94, and a light chain variable region comprising the amino acid sequence of SEQ ID NO: 107.

In some embodiments, an antibody of the present application cross-competes for binding to human CTLA4 with an antibody comprising: a) an HVR-H1 comprising an amino acid sequence selected from SEQ ID NOS: 18-29; an HVR-H2 comprising an amino acid sequence selected from SEQ ID NOS: 30-39; and an HVR-H3 comprising an amino acid sequence selected from SEQ ID NOS: 40-52; and/or b) an HVR-L1 comprising an amino acid sequence selected from SEQ ID NOS: 53-65; an HVR-L2 comprising an amino acid sequence selected from SEQ ID NOS: 66-69; and an HVR-L3 comprising an amino acid sequence selected from SEQ ID NOS: 70-81. In some embodiments, an antibody of the present application cross-competes for binding to human CTLA4 with an antibody comprising one, two, three, four, five, or all six of the HVRs shown for any of the exemplary antibodies described in Table A. In some embodiments, an antibody of the present application cross-competes for binding to human CTLA4 with an antibody comprising: a) a heavy chain variable region comprising an amino acid sequence selected from SEQ ID NOS: 82-94; and/or b) a light chain variable region comprising an amino acid sequence selected from SEQ ID NOS: 95-107. In some embodiments, an antibody of the present application cross-competes for binding to human CTLA4 with an antibody comprising a VH and/or VL shown for any of the exemplary antibodies described in Table B.

The CTLA4 antibodies described herein may be in any class, such as IgG, IgM, IgE, IgA, or IgD. In some embodiments, the CTLA4 antibodies are in the IgG class, such as IgG1, IgG2, IgG3, or IgG4 subclass. A CTLA4 antibody can be converted from one class or subclass to another class or subclass using methods known in the art. An exemplary method for producing an antibody in a desired class or subclass comprises the steps of isolating a nucleic acid encoding a heavy chain of a CTLA4 antibody and a nucleic acid encoding a light chain of a CTLA4 antibody, isolating the sequence encoding the V_{H} region, ligating the V_{H} sequence to a sequence encoding a heavy chain constant region of the desired class or subclass, expressing the light chain gene and the heavy chain construct in a cell, and collecting the CTLA4 antibody. Antibodies of the present application may be monoclonal antibodies or polyclonal antibodies. Antibodies of the present application may be monospecific antibodies or multispecific (e.g., bispecific, trispecific, etc.) antibodies. In some embodiments, the CTLA4 antibodies described herein may include one or more Fc mutations (e.g., that modulate (increase or decrease) ADCC or CDC activities). Any suitable Fc mutations known in the art may be used in the CTLA4 antibodies of the present application.

In some embodiments, the anti-CTLA4 antibody comprises a heavy chain comprising the amino acid sequence EVQLVESGGGLVQPGGSLRLSCAASGYSISSGYHWSWIRQAPGKGLEWLARIDWDDDKYYSTSL KSRLTISRDNSKNTLYLQLNSLRAEDTAVYYCARSYVYFDYWGQGTLVTVSSASTKGPSVFPLA PSSKSTSGGTAALGCLVKDYFPEPVTVSWNSGALTSGVHTFPAVLQSSGLYSLSSVVTVPSSSL GTQTYICNVNHKPSNTKVDKKVEPKSCDKTHTCPPCPAPELLGGPSVFLFPPKPKDTLMISRTP EVTCVVVDVSHEDPEVKFNWYVDGVEVHNAKTKPREEQYNSTYRVVSVLTVLHQDWLNGKEYKC KVSNKALPAPIEKTISKAKGQPREPQVYTLPPSRDELTKNQVSLTCLVKGFYPSDIAVEWESNG QPENNYKTTPPVLDSDGSFFLYSKLTVDKSRWQQGNVFSCSVMHEALHNHYTQKSLSLSPG (SEQ ID NO:125) and a light chain comprising the amino acid sequence DIQLTQSPSSLSASVGDRVTITCRASQSVRGRFLAWYQQKPGKAPKLLIYDASNRATGIPSRFS GSGSGTDFTLTISSLQPEDFATYYCQQSSSWPPTFGQGTKVEIKRTVAAPSVFIFPPSDEQLKS GTASVVCLLNNFYPREAKVQWKVDNALQSGNSQESVTEQDSKDSTYSLSSTLTLSKADYEKHKV YACEVTHQGLSSPVTKSFNRGEC (SEQ ID NO:127). In some embodiments, the anti-CTLA4 antibody comprises a heavy chain comprising the amino acid sequence EVQLVESGGGLVQPGGSLRLSCAASGYSISSGYHWSWIRQAPGKGLEWLARIDWDDDKYYSTSL KSRLTISRDNSKNTLYLQLNSLRAEDTAVYYCARSYVYFDYWGQGTLVTVSSASTKGPSVFPLA PSSKSTSGGTAALGCLVKDYFPEPVTVSWNSGALTSGVHTFPAVLQSSGLYSLSSVVTVPSSSL GTQTYICNVNHKPSNTKVDKKVEPKSCDKTHTCPPCPAPELLGGPSVFLFPPKPKDTLMISRTP EVTCVVVDVSHEDPEVKFNWYVDGVEVHNAKTKPREEQYNSTYRVVSVLTVLHQDWLNGKEYKC KVSNKALPAPIEKTISKAKGQPREPQVYTLPPSRDELTKNQVSLTCLVKGFYPSDIAVEWESNG QPENNYKTTPPVLDSDGSFFLYSKLTVDKSRWQQGNVFSCSVMHEALHNHYTQKSLSLSPGK (SEQ ID NO:126) and a light chain comprising the amino acid sequence DIQLTQSPSSLSASVGDRVTITCRASQSVRGRFLAWYQQKPGKAPKLLIYDASNRATGIPSRFS GSGSGTDFTLTISSLQPEDFATYYCQQSSSWPPTFGQGTKVEIKRTVAAPSVFIFPPSDEQLKS GTASVVCLLNNFYPREAKVQWKVDNALQSGNSQESVTEQDSKDSTYSLSSTLTLSKADYEKHKV YACEVTHQGLSSPVTKSFNRGEC (SEQ ID NO:127). In some embodiments, the anti-CTLA4 antibody refers to a mix of antibody species, wherein each antibody species has a light chain comprising the amino acid sequence of SEQ ID NO: 127 and a heavy chain comprising either the amino acid sequence of SEQ ID NO: 125 or 126.

In some embodiments, the anti-CTLA4 antibody comprises a heavy chain comprising the amino acid sequence EVQLVESGGGLVQPGGSLRLSCAASGFTFSDYAIHWVRQAPGKGLEWIGIISPSGGSTKYAQKF QGRVTISRDNSKNTLYLQLNSLRAEDTAVYYCARLGYGYFDVWGQGTLVTVSSASTKGPSVFPL APSSKSTSGGTAALGCLVKDYFPEPVTVSWNSGALTSGVHTFPAVLQSSGLYSLSSVVTVPSSS LGTQTYICNVNHKPSNTKVDKKVEPKSCDKTHTCPPCPAPELLGGPSVFLFPPKPKDTLMISRT PEVTCVVVDVSHEDPEVKFNWYVDGVEVHNAKTKPREEQYNSTYRVVSVLTVLHQDWLNGKEYK CKVSNKALPAPIEKTISKAKGQPREPQVYTLPPSRDELTKNQVSLTCLVKGFYPSDIAVEWESN GQPENNYKTTPPVLDSDGSFFLYSKLTVDKSRWQQGNVFSCSVMHEALHNHYTQKSLSLSPG (SEQ ID NO:128) and a light chain comprising the amino acid sequence DIQLTQSPSSLSASVGDRVTITCRASQSVDFYGISFLHWYQQKPGKAPKLLIYDASSLESGVPS RFSGSGSGTDFTLTISSLQPEDFATYYCVQALQLPLTFGQGTKVEIKRTVAAPSVFIFPPSDEQ LKSGTASVVCLLNNFYPREAKVQWKVDNALQSGNSQESVTEQDSKDSTYSLSSTLTLSKADYEK HKVYACEVTHQGLSSPVTKSFNRGEC (SEQ ID NO:130). In some embodiments, the anti-CTLA4 antibody comprises a heavy chain comprising the amino acid sequence EVQLVESGGGLVQPGGSLRLSCAASGFTFSDYAIHWVRQAPGKGLEWIGIISPSGGSTKYAQKF QGRVTISRDNSKNTLYLQLNSLRAEDTAVYYCARLGYGYFDVWGQGTLVTVSSASTKGPSVFPL APSSKSTSGGTAALGCLVKDYFPEPVTVSWNSGALTSGVHTFPAVLQSSGLYSLSSVVTVPSSS LGTQTYICNVNHKPSNTKVDKKVEPKSCDKTHTCPPCPAPELLGGPSVFLFPPKPKDTLMISRT PEVTCVVVDVSHEDPEVKFNWYVDGVEVHNAKTKPREEQYNSTYRVVSVLTVLHQDWLNGKEYK CKVSNKALPAPIEKTISKAKGQPREPQVYTLPPSRDELTKNQVSLTCLVKGF-YPSDIAVEWESN GQPENNYKTTPPVLDSDGSFFLYSKLTVDKSRWQQGNVFSCSVMHEALHNHYTQKSLSLSPGK (SEQ ID NO:129) and a light chain comprising the amino acid sequence DIQLTQSPSSLSASVGDRVTITCRASQSVDFYGISFLHWYQQKPGKAPKLLIYDASSLESGVPS RFSGSGSGTDFTLTISSLQPEDFATYYCVQALQLPLTFGQGTKVEIKRTVAAPSVFIFPPSDEQ LKSGTASVVCLLNNFYPREAKVQWKVDNALQSGNSQESVTEQDSKDSTYSLSSTLTLSKADYEK HKVYACEVTHQGLSSPVTKSFNRGEC (SEQ ID NO:130). In some embodiments, the anti-CTLA4 antibody refers to a mix of antibody species, wherein each antibody species has a light chain comprising the amino acid sequence of SEQ ID NO: 130 and a heavy chain comprising either the amino acid sequence of SEQ ID NO: 128 or 129.

In some embodiments, the anti-CTLA4 antibody comprises a heavy chain comprising the amino acid sequence EVQLVESGGGLVQPGGSLRLSCAASGYSITSGYYWAWIRQAPGKGLEWVSSISGSGSTTYYADS VKGRFTISRDNSKNTLYLQLNSLRAEDTAVYYCARDGFGYFDYWGQGTLVTVSSASTKGPSVFP LAPSSKSTSGGTAALGCLVKDYFPEPVTVSWNSGALTSGVHTFPAVLQSSGLYSLSSVVTVPSS SLGTQTYICNVNHKPSNTKVDKKVEPKSCDKTHTCPPCPAPELLGGPSVFLFPPKPKDTLMISR TPEVTCVVVDVSHEDPEVKFNWYVDGVEVHNAKTKPREEQYNSTYRVVSVLTVLHQDWLNGKEY KCKVSNKALPAPIEKTISKAKGQPREPQVYTLPPSRDELTKNQVSLTCLVKGFYPSDIAVEWES NGQPENNYKTTPPVLDSDGSFFLYSKLTVDKSRWQQGNVFSCSVMHEALHNHYTQKSLSLSPG (SEQ ID NO:131) and a light chain comprising the amino acid sequence DIQLTQSPSSLSASVGDRVTITCSASSSVSYVYWYQQKPGKAPKLLIYDASSLESGVPSRFSGS GSGTDFTLTISSLQPEDFATYYCVQGLQTPWTFGQGTKVEIKRTVAAPSVFIFPPSDEQLKSGT ASVVCLLNNFYPREAKVQWKVDNALQSGNSQESVTEQDSKDSTYSLSSTLTLSKADYEKHKVYA CEVTHQGLSSPVTKSFNRGEC (SEQ ID NO:133). In some embodiments, the anti-CTLA4 antibody comprises a heavy chain comprising the amino acid sequence EVQLVESGGGLVQPGGSLRLSCAASGYSITSGYYWAWIRQAPGKGLEWVSSISGSGSTTYYADS VKGRFTISRDNSKNTLYLQLNSLRAEDTAVYYCARDGFGYFDYWGQGTLVTVSSASTKGPSVFP LAPSSKSTSGGTAALGCLVKDYFPEPVTVSWNSGALTSGVHTFPAVLQSSGLYSLSSVVTVPSS SLGTQTYICNVNHKPSNTKVDKKVEPKSCDKTHTCPPCPAPELLGGPSVFLFPPKPKDTLMISR TPEVTCVVVDVSHEDPEVKFNWYVDGVEVHNAKTKPREEQYNSTYRVVSVLTVLHQDWLNGKEY KCKVSNKALPAPIEKTISKAKGQPREPQVYTLPPSRDELTKNQVSLTCLVKGFYPSDIAVEWES NGQPENNYKTTPPVLDSDGSFFLYSKLTVDKSRWQQGNVFSCSVMHEALHNHYTQKSLSLSPGK (SEQ ID NO:132) and a light chain comprising the amino acid sequence DIQLTQSPSSLSASVGDRVTITCSASSSVSYVYWYQQKPGKAPKLLIYDASSLESGVPSRFSGS GSGTDFTLTISSLQPEDFATYYCVQGLQTPWTFGQGTKVEIKRTVAAPSVFIFPPSDEQLKSGT ASVVCLLNNFYPREAKVQWKVDNALQSGNSQESVTEQDSKDSTYSLSSTLTLSKADYEKHKVYA CEVTHQGLSSPVTKSFNRGEC (SEQ ID NO:133). In some embodiments, the anti-CTLA4 antibody refers to a mix of antibody species, wherein each antibody species has a light chain comprising the amino acid sequence of SEQ ID NO: 133 and a heavy chain comprising either the amino acid sequence of SEQ ID NO: 131 or 132.

In some embodiments, the anti-CTLA4 antibody comprises a heavy chain comprising the amino acid sequence EVQLVESGGGLVQPGGSLRLSCAASGFTFSGYAIHWVRQAPGKGLEWIGIISPSGGGTKYAQKF QGRVTISRDNSKNTLYLQLNSLRAEDTAVYYCARHPFAYWGQGTLVTVSSASTKGPSVFPLAPS SKSTSGGTAALGCLVKDYFPEPVTVSWNSGALTSGVHTFPAVLQSSGLYSLSSVVTVPSSSLGT QTYICNVNHKPSNTKVDKKVEPKSCDKTHTCPPCPAPELLGGPSVFLFPPKPKDTLMISRTPEV TCVVVDVSHEDPEVKFNWYVDGVEVHNAKTKPREEQYNSTYRVVSVLTVLHQDWLNGKEYKCKV SNKALPAPIEKTISKAKGQPREPQVYTLPPSRDELTKNQVSLTCLVKGFYPSDIAVEWESNGQP ENNYKTTPPVLDSDGSFFLYSKLTVDKSRWQQGNVFSCSVMHEALHNHYTQKSLSLSPG (SEQ ID NO:134) and a light chain comprising the amino acid sequence DIQLTQSPSSLSASVGDRVTITCRASQSVDFYGISFLDWYQQKPGKAPKLLIYDASNRATGIPS RFSGSGSGTDFTLTISSLQPEDFATYYCQQYVSSPPEYTFGQGTKVEIKRTVAAPSVFIFPPSD EQLKSGTASVVCLLNNFYPREAKVQWKVDNALQSGNSQESVTEQDSKDSTYSLSSTLTLSKADY EKHKVYACEVTHQGLSSPVTKSFNRGEC (SEQ ID NO:136). In some embodiments, the anti-CTLA4 antibody comprises a heavy chain comprising the amino acid sequence EVQLVESGGGLVQPGGSLRLSCAASGFTFSGYAIHWVRQAPGKGLEWIGIISPSGGGTKYAQKF QGRVTISRDNSKNTLYLQLNSLRAEDTAVYYCARHPFAYWGQGTLVTVSSASTKGPSVFPLAPS SKSTSGGTAALGCLVKDYFPEPVTVSWNSGALTSGVHTFPAVLQSSGLYSLSSVVTVPSSSLGT QTYICNVNHKPSNTKVDKKVEPKSCDKTHTCPPCPAPELLGGPSVFLFPPKPKDTLMISRTPEV TCVVVDVSHEDPEVKFNWYVDGVEVHNAKTKPREEQYNSTYRVVSVLTVLHQDWLNGKEYKCKV SNKALPAPIEKTISKAKGQPREPQVYTLPPSRDELTKNQVSLTCLVKGFYPSDIAVEWESNGQP ENNYKTTPPVLDSDGSFFLYSKLTVDKSRWQQGNVFSCSVMHEALHNHYTQKSLSLSPGK (SEQ ID NO:135) and a light chain comprising the amino acid sequence DIQLTQSPSSLSASVGDRVTITCRASQSVDFYGISFLDWYQQKPGKAPKLLIYDASNRATGIPS RFSGSGSGTDFTLTISSLQPEDFATYYCQQYVSSPPEYTFGQGTKVEIKRTVAAPSVFIFPPSD EQLKSGTASVVCLLNNFYPREAKVQWKVDNALQSGNSQESVTEQDSKDSTYSLSSTLTLSKADY EKHKVYACEVTHQGLSSPVTKSFNRGEC (SEQ ID NO:136). In some embodiments, the anti-CTLA4 antibody refers to a mix of antibody species, wherein each antibody species has a light chain comprising the amino acid sequence of SEQ ID NO: 136 and a heavy chain comprising either the amino acid sequence of SEQ ID NO: 134 or 135.

In some embodiments, the anti-CTLA4 antibody comprises a heavy chain comprising the amino acid sequence EVQLVESGGGLVQPGGSLRLSCAASGYTFSGYAIHWVRQAPGKGLEWIGIISPSGGGTKYAQKF QGRVTISRDNSKNTLYLQLNSLRAEDTAVYYCARLYDVAYWGQGTLVTVSSASTKGPSVFPLAP SSKSTSGGTAALGCLVKDYFPEPVTVSWNSGALTSGVHTFPAVLQSSGLYSLSSVVTVPSSSLG TQTYICNVNHKPSNTKVDKKVEPKSCDKTHTCPPCPAPELLGGPSVFLFPPKPKDTLMISRTPE VTCVVVDVSHEDPEVKFNWYVDGVEVHNAKTKPREEQYNSTYRVVSVLTVLHQDWLNGKEYKCK VSNKALPAPIEKTISKAKGQPREPQVYTLPPSRDELTKNQVSLTCLVKGFYPSDIAVEWESNGQ PENNYKTTPPVLDSDGSFFLYSKLTVDKSRWQQGNVFSCSVMHEALHNHYTQKSLSLSPG (SEQ ID NO:137) and a light chain comprising the amino acid sequence DIQLTQSPSSLSASVGDRVTITCRASQSVDFHGKSFLHWYQQKPGKAPKLLIYDASSLESGVPS RFSGSGSGTDFTLTISSLQPEDFATYYCEQSLEVPFTFGQGTKVEIKRTVAAPSVFIFPPSDEQ LKSGTASVVCLLNNFYPREAKVQWKVDNALQSGNSQESVTEQDSKDSTYSLSSTLTLSKADYEK HKVYACEVTHQGLSSPVTKSFNRGEC (SEQ ID NO:139). In some embodiments, the anti-CTLA4 antibody comprises a heavy chain comprising the amino acid sequence EVQLVESGGGLVQPGGSLRLSCAASGYTFSGYAIHWVRQAPGKGLEWIGIISPSGGGTKYAQKF QGRVTISRDNSKNTLYLQLNSLRAEDTAVYYCARLYDVAYWGQGTLVTVSSASTKGPSVFPLAP SSKSTSGGTAALGCLVKDYFPEPVTVSWNSGALTSGVHTFPAVLQSSGLYSLSSVVTVPSSSLG TQTYICNVNHKPSNTKVDKKVEPKSCDKTHTCPPCPAPELLGGPSVFLFPPKPKDTLMISRTPE VTCVVVDVSHEDPEVKFNWYVDGVEVHNAKTKPREEQYNSTYRVVSVLTVLHQDWLNGKEYKCK VSNKALPAPIEKTISKAKGQPREPQVYTLPPSRDELTKNQVSLTCLVKGFYPSDIAVEWESNGQ PENNYKTTPPVLDSDGSFFLYSKLTVDKSRWQQGNVFSCSVMHEALHNHYTQKSLSLSPGK (SEQ ID NO:138) and a light chain comprising the amino acid sequence DIQLTQSPSSLSASVGDRVTITCRASQSVDFHGKSFLHWYQQKPGKAPKLLIYDASSLESGVPS RFSGSGSGTDFTLTISSLQPEDFATYYCEQSLEVPFTFGQGTKVEIKRTVAAPSVFIFPPSDEQ LKSGTASVVCLLNNFYPREAKVQWKVDNALQSGNSQESVTEQDSKDSTYSLSSTLTLSKADYEK HKVYACEVTHQGLSSPVTKSFNRGEC (SEQ ID NO:139). In some embodiments, the anti-CTLA4 antibody refers to a mix of antibody species, wherein each antibody species has a light chain comprising the amino acid sequence of SEQ ID NO: 139 and a heavy chain comprising either the amino acid sequence of SEQ ID NO: 137 or 138.

In some embodiments, the anti-CTLA4 antibody is an antigen-binding fragment of an anti-CTLA4 antibody. the antigen-binding fragments of a CTLA4 antibody include: (i) a Fab fragment, which is a monovalent fragment consisting of the V_{L}, V_{H}, C_{L} and C_{H}1 domains; (ii) a F(ab′)₂ fragment, which is a bivalent fragment comprising two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody; (v) a dAb fragment (Ward et al., (1989) Nature 341:544-546), which consists of a V_{H} domain; (vi) an isolated CDR, and (vii) single chain antibody (scFv), which is a polypeptide comprising a V_{L} region of an antibody linked to a V_{H} region of an antibody (see e.g., Bird et al. (1988) Science 242:423-426; Huston et al. (1988) Proc. Natl. Acad. Sci. USA 85:5879-5883).

In some embodiments, the anti-CTLA4 antibody is a derivative of any one of the anti-CTLA4 antibodies described herein. In some embodiments, the antibody derivative is derived from modifications of the amino acid sequences of an illustrative antibody (e.g., a “parent antibody”) of the present application while conserving the overall molecular structure of the parent antibody amino acid sequence. Amino acid sequences of any regions of the parent antibody chains may be modified, such as framework regions, HVR regions, or constant regions. Types of modifications include substitutions, insertions, deletions, or combinations thereof, of one or more amino acids of the parent antibody.

In some embodiments, the antibody derivative comprises a V_{L} or V_{H} region that is at least 65%, at least 75%, at least 85%, at least 90%, at least 91%, at least 92%, at least 93%, at least 94%, at least 95%, at least 96%, at least 97%, at least 98%, or at least 99% identical to an amino acid sequence as set forth in any of SEQ ID NOS: 82-107 In some embodiments, the antibody derivative comprises an HVR-H1 amino acid sequence region that is at least 65%, at least 75%, at least 85%, at least 90%, at least 91%, at least 92%, at least 93%, at least 94%, at least 95%, at least 96%, at least 97%, at least 98%, or at least 99% identical to an amino acid sequence as set forth in any of SEQ ID NOS: 18-29. In some embodiments, the antibody derivative comprises an HVR-H2 amino acid sequence region that is at least 65%, at least 75%, at least 85%, at least 90%, at least 91%, at least 92%, at least 93%, at least 94%, at least 95%, at least 96%, at least 97%, at least 98%, or at least 99% identical to an amino acid sequence as set forth in any of SEQ ID NOS: 30-39. In some embodiments, the antibody derivative comprises an HVR-H3 amino acid sequence region that is at least 65%, at least 75%, at least 85%, at least 90%, at least 91%, at least 92%, at least 93%, at least 94%, at least 95%, at least 96%, at least 97%, at least 98%, or at least 99% identical to an amino acid sequence as set forth in any of SEQ ID NOS: 40-52. In some embodiments, the antibody derivative comprises an HVR-L1 amino acid sequence region that is at least 65%, at least 75%, at least 85%, at least 90%, at least 91%, at least 92%, at least 93%, at least 94%, at least 95%, at least 96%, at least 97%, at least 98%, or at least 99% identical to an amino acid sequence as set forth in any of SEQ ID NOS: 53-65. In some embodiments, the antibody derivative comprises an HVR-L2 amino acid sequence region that is at least 65%, at least 75%, at least 85%, at least 90%, at least 91%, at least 92%, at least 93%, at least 94%, at least 95%, at least 96%, at least 97%, at least 98%, or at least 99% identical to an amino acid sequence as set forth in any of SEQ ID NOS: 66-69. In some embodiments, the antibody derivative comprises an HVR-L3 amino acid sequence region that is at least 65%, at least 75%, at least 85%, at least 90%, at least 91%, at least 92%, at least 93%, at least 94%, at least 95%, at least 96%, at least 97%, at least 98%, or at least 99% identical to an amino acid sequence as set forth in any of SEQ ID NOS: 70-81.

In some particular embodiments, the derivative comprises 1, 2, 3, 4, 5, 6, 7, 8, 9, 10, 11, 12, 13, 14, or 15 conservative or non-conservative substitutions, and/or 1, 2, 3, 4, 5, 6, 7, 8, 9, 10, 11, 12, 13, 14, or 15 additions and/or deletions to an amino acid sequence as set forth in any of SEQ ID NOS: 18-107.

Amino acid substitutions encompass both conservative substitutions and non-conservative substitutions. The term “conservative amino acid substitution” means a replacement of one amino acid with another amino acid where the two amino acids have similarity in certain physico-chemical properties such as polarity, charge, solubility, hydrophobicity, hydrophilicity, and/or the amphipathic nature of the residues involved. For example, substitutions typically may be made within each of the following groups: (a) nonpolar (hydrophobic) amino acids, such as alanine, leucine, isoleucine, valine, proline, phenylalanine, tryptophan, and methionine; (b) polar neutral amino acids, such as glycine, serine, threonine, cysteine, tyrosine, asparagine, and glutamine; (c) positively charged (basic) amino acids, such as arginine, lysine, and histidine; and (d) negatively charged (acidic) amino acids, such as aspartic acid and glutamic acid.

The modifications may be made in any positions of the amino acid sequences of the antibody, including the HVRs, framework regions, or constant regions. In one embodiment, the present application provides an antibody derivative that contains the V_{H} and V_{L} HVR sequences of an illustrative antibody of this disclosure, yet contains framework sequences different from those of the illustrative antibody. Such framework sequences can be obtained from public DNA databases or published references that include germline antibody gene sequences. For example, germline DNA sequences for human heavy and light chain variable region genes can be found in the Genbank database or in the “VBase” human germline sequence database (Kaba et al., Sequences of Proteins of Immunological Interest, Fifth Edition, U.S. Department of Health and Human Services, NIH Publication No. 91-3242 (1991); Tomlinson et al., J. Mol. Biol. 227:776-798 (1992); and Cox et al., Eur. J. Immunol. 24:827-836 (1994)). Framework sequences that may be used in constructing an antibody derivative include those that are structurally similar to the framework sequences used by illustrative antibodies of the disclosure For example, the HVR-H1, HVR-H2, and HVR-H3 sequences, and the HVR-L1, HVR-L2, and HVR-L3 sequences of an illustrative antibody can be grafted onto framework regions that have the identical sequence as that found in the germline immunoglobulin gene from which the framework sequence derive, or the HVR sequences can be grafted onto framework regions that contain one or more mutations as compared to the germline sequences.

In some embodiments, the antibody derivative is a chimeric antibody, which comprises an amino acid sequence of an illustrative antibody of the disclosure. In one example, one or more HVRs from one or more illustrative antibodies are combined with HVRs from an antibody from a non-human animal, such as mouse or rat. In another example, all of the HVRs of the chimeric antibody are derived from one or more illustrative antibodies. In some particular embodiments, the chimeric antibody comprises one, two, or three HVRs from the heavy chain variable region and/or one, two, or three HVRs from the light chain variable region of an illustrative antibody. Chimeric antibodies can be generated using conventional methods known in the art.

Another type of modification is to mutate amino acid residues within the HVR regions of the V_{H} and/or V_{L} chain. Site-directed mutagenesis or PCR-mediated mutagenesis can be performed to introduce the mutation(s) and the effect on antibody binding, or other functional property of interest, can be evaluated in in vitro or in vivo assays known in the art. Typically, conservative substitutions are introduced. The mutations may be amino acid additions and/or deletions. Moreover, typically no more than one, two, three, four or five residues within an HVR region are altered. In some embodiments, the antibody derivative comprises 1, 2, 3, or 4 amino acid substitutions in the heavy chain HVRs and/or in the light chain HVRs. In another embodiment, the amino acid substitution is to change one or more cysteines in an antibody to another residue, such as, without limitation, alanine or serine. The cysteine may be a canonical or non-canonical cysteine. In one embodiment, the antibody derivative has 1, 2, 3, or 4 conservative amino acid substitutions in the heavy chain HVR regions relative to the amino acid sequences of an illustrative antibody.

Modifications may also be made to the framework residues within the V_{H} and/or V_{L} regions. Typically, such framework variants are made to decrease the immunogenicity of the antibody. One approach is to “back mutate” one or more framework residues to the corresponding germline sequence. An antibody that has undergone somatic mutation may contain framework residues that differ from the germline sequence from which the antibody is derived. Such residues can be identified by comparing the antibody framework sequences to the germline sequences from which the antibody is derived. To return the framework region sequences to their germline configuration, the somatic mutations can be “back mutated” to the germline sequence by, for example, site-directed mutagenesis or PCR-mediated mutagenesis.

In addition, modifications may also be made within the Fc region of an illustrative antibody, typically to alter one or more functional properties of the antibody, such as serum half-life, complement fixation, Fc receptor binding, and/or antigen-dependent cellular cytotoxicity. In one example, the hinge region of CH1 is modified such that the number of cysteine residues in the hinge region is altered, e.g., increased or decreased. This approach is described further in U.S. Pat. No. 5,677,425. The number of cysteine residues in the hinge region of CH1 is altered to, for example, facilitate assembly of the light and heavy chains or to increase or decrease the stability of the antibody. In another case, the Fc hinge region of an antibody is mutated to decrease the biological half-life of the antibody.

Furthermore, an antibody of the present application may be modified to alter its potential glycosylation site or pattern in accordance with routine experimentation known in the art. In another aspect, the present application provides a derivative of a CTLA4 antibody that contains at least one mutation in a variable region of a light chain or heavy chain that changes the pattern of glycosylation in the variable region. Such an antibody derivative may have an increased affinity and/or a modified specificity for binding an antigen. The mutations may add a novel glycosylation site in the V region, change the location of one or more V region glycosylation site(s), or remove a pre-existing V region glycosylation site. In one embodiment, the present application provides a derivative of a CTLA4 antibody having a potential N-linked glycosylation site at asparagine in the heavy chain variable region, wherein the potential N-linked glycosylation site in one heavy chain variable region is removed. In another embodiment, the present application provides a derivative of a CTLA4 antibody having a potential N-linked glycosylation site at asparagine in the heavy chain variable region, wherein the potential N-linked glycosylation site in both heavy chain variable regions is removed. Method of altering the glycosylation pattern of an antibody is known in the art, such as those described in U.S. Pat. No. 6,933,368, the disclosure of which incorporated herein by reference.

### Methods of Making

Antibodies of the present application can be produced by techniques known in the art, including conventional monoclonal antibody methodology e.g., the standard somatic cell hybridization technique (See e.g., Kohler and Milstein, Nature 256:495 (1975), viral or oncogenic transformation of B lymphocytes, or recombinant antibody technologies.

Hybridoma production is a very well-established procedure. The common animal system for preparing hybridomas is the murine system. Immunization protocols and techniques for isolation of immunized splenocytes for fusion are known in the art. Fusion partners (e.g., murine myeloma cells) and fusion procedures are also known. One well-known method that may be used for making human CTLA4 antibodies provided by the present application involves the use of a XenoMouse™ animal system. XenoMouse™ mice are engineered mouse strains that comprise large fragments of human immunoglobulin heavy chain and light chain loci and are deficient in mouse antibody production (see e.g., Green et al., (1994) Nature Genetics 7:13-21; WO2003/040170). The animal is immunized with a CTLA4 antigen. The CTLA4 antigen is isolated and/or purified CTLA4. It may be a fragment of CTLA4, such as the extracellular domain of CTLA4. Immunization of animals can be carried out by any method known in the art (see e.g., Harlow and Lane, Antibodies: A Laboratory Manual, New York: Cold Spring Harbor Press, 1990). Methods for immunizing non-human animals such as mice, rats, sheep, goats, pigs, cattle and horses are well known in the art (see e.g., Harlow and Lane, supra, and U.S. Pat. No. 5,994,619). The CTLA4 antigen may be administered with an adjuvant to stimulate the immune response. Exemplary adjuvants include complete or incomplete Freund's adjuvant, RIBI (muramyl dipeptides) or ISCOM (immunostimulating complexes). After immunization of an animal with a CTLA4 antigen, antibody-producing immortalized cell lines are prepared from cells isolated from the immunized animal. After immunization, the animal is sacrificed and lymph node and/or splenic B cells are immortalized. Methods of immortalizing cells include, but are not limited to, transferring them with oncogenes, inflecting them with the oncogenic virus cultivating them under conditions that select for immortalized cells, subjecting them to carcinogenic or mutating compounds, fusing them with an immortalized cell, e.g., a myeloma cell, and inactivating a tumor suppressor gene (see e.g., Harlow and Lane, supra). If fusion with myeloma cells is used, the myeloma cells preferably do not secrete immunoglobulin polypeptides (a non-secretory cell line). Immortalized cells are screened using CTLA4, a portion thereof, or a cell expressing CTLA4. CTLA4 antibody-producing cells, e.g., hybridomas, are selected, cloned and further screened for desirable characteristics, including robust growth, high antibody production and desirable antibody characteristics, as discussed further below. Hybridomas can be expanded in vivo in syngeneic animals, in animals that lack an immune system, e.g., nude mice, or in cell culture in vitro. Methods of selecting, cloning and expanding hybridomas are well known to those of ordinary skill in the art.

Antibodies of the present application may also be prepared using phage display or yeast display methods. Such display methods for isolating human antibodies are established in the art (see e.g., Knappik, et al. (2000) J. Mol. Biol. 296, 57-86; Feldhaus et al. (2003) Nat Biotechnol 21:163-170).

In some embodiments, the anti-CTLA4 antibody is prepared by expressing one or more nucleic acids encoding the anti-CTLA4 antibody or polypeptide chains thereof in a host cell. In some embodiments, the one or more nucleic acids is a DNA or RNA, and may or may not contain intronic sequences. Typically, the nucleic acid is a cDNA molecule.

Nucleic acids of the disclosure can be obtained using any suitable molecular biology techniques. For antibodies expressed by hybridomas, cDNAs encoding the light and heavy chains of the antibody made by the hybridoma can be obtained by PCR amplification or cDNA cloning techniques. For antibodies obtained from an immunoglobulin gene library (e.g., using phage display techniques), the nucleic acid encoding the antibody can be recovered from the library.

The isolated DNA encoding the VH region can be converted to a full-length heavy chain gene by operatively linking the VH-encoding DNA to another DNA molecule encoding heavy chain constant regions (CH1, CH2 and CH3). The sequences of human heavy chain constant region genes are known in the art (see e.g., Kabat et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The heavy chain constant region can be an IgG1, IgG2, IgG3, IgG4, IgA, IgE, IgM or IgD constant region, but most preferably is an IgG4 or IgG2 constant region without ADCC effect. The IgG4 constant region sequence can be any of the various alleles or allotypes known to occur among different individuals. These allotypes represent naturally occurring amino acid substitution in the IgG4 constant regions. For a Fab fragment heavy chain gene, the VH-encoding DNA can be operatively linked to another DNA molecule encoding only the heavy chain CH1 constant region.

The isolated DNA encoding the VL region can be converted to a full-length light chain gene (as well as a Fab light chain gene) by operatively linking the VL-encoding DNA to another DNA molecule encoding the light chain constant region, CL. The sequences of human light chain constant region genes are known in the art (see e.g., Kabat et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The light chain constant region can be a kappa or lambda constant region.

To create a scFv gene, the VH- and VL-encoding DNA fragments are operatively linked to another fragment encoding a flexible linker, e.g., encoding the amino acid sequence (Gly₄-Ser)₃, such that the VH and VL sequences can be expressed as a contiguous single-chain protein, with the VL and VH regions joined by the flexible linker (see e.g., Bird et al., Science 242:423-426 (1988); Huston et al., Proc. Natl. Acad. Sci. USA 85:5879-5883 (1988); and McCafferty et al., Nature 348:552-554 (1990)).

In some embodiments, there is provided a vector that comprises a nucleic acid molecule described herein. In some embodiments, the vector is an expression vector or a display vector (e.g., a viral display vector, a bacterial display vector, a yeast display vector, an insect display vector, a mammalian display vector, etc.). The nucleic acid molecule may encode a portion of a light chain or heavy chain (such as a CDR or a HVR; a light or heavy chain variable region), a full-length light or heavy chain, polypeptide that comprises a portion or full-length of a heavy or light chain, or an amino acid sequence of an antibody derivative or antigen-binding fragment. In some embodiments, the vector is an expression vector useful for the expression of an anti-CTLA4 antibody. In some embodiments, provided herein are vectors, wherein a first vector comprises a polynucleotide sequence encoding a heavy chain variable region as described herein, and a second vector comprises a polynucleotide sequence encoding a light chain variable region as described herein. In some embodiments, a single vector comprises polynucleotides encoding a heavy chain variable region as described herein and a light chain variable region as described herein.

To express an anti-CTLA4 antibody of the disclosure, DNAs encoding partial or full-length light and heavy chains are inserted into expression vectors such that the DNA molecules are operatively linked to transcriptional and translational control sequences. In this context, the term “operatively linked” means that an antibody gene is ligated into a vector such that transcriptional and translational control sequences within the vector serve their intended function of regulating the transcription and translation of the DNA molecule. The expression vector and expression control sequences are chosen to be compatible with the expression host cell used. The antibody light chain gene and the antibody heavy chain gene can be inserted into separate vectors, or both genes can be inserted into the same expression vector. The antibody genes are inserted into the expression vector by any suitable methods (e.g., ligation of complementary restriction sites on the antibody gene fragment and vector, or homologous recombination-based DNA ligation). The light and heavy chain variable regions of the antibodies described herein can be used to create full-length antibody genes of any antibody isotype and subclass by inserting them into expression vectors already encoding heavy chain constant and light chain constant regions of the desired isotype and subclass such that the VH segment is operatively linked to the CH segment(s) within the vector and the VL segment is operatively linked to the CL segment within the vector. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody sequences, the expression vectors of the disclosure typically carry regulatory sequences that control the expression of the antibody sequences in a host cell. The term “regulatory sequence” is intended to include promoters, enhancers and other expression control elements (e.g., polyadenylation signals) that control the transcription or translation of the antibody chain genes. Such regulatory sequences are described, for example, in Goeddel (Gene Expression Technology. Methods in Enzymology 185, Academic Press, San Diego, Calif. (1990)). It will be appreciated by those skilled in the art that the design of the expression vector, including the selection of regulatory sequences, may depend on such factors as the choice of the host cell to be transformed, the level of expression of protein desired, etc. Examples of regulatory sequences for mammalian host cell expression include viral elements that direct high levels of protein expression in mammalian cells, such as promoters and/or enhancers derived from cytomegalovirus (CMV), Simian Virus 40 (SV40), adenovirus, (e.g., the adenovirus major late promoter (AdMLP) and polyoma. Alternatively, nonviral regulatory sequences may be used, such as the ubiquitin promoter or β-globin promoter. Still further, regulatory elements composed of sequences from different sources, such as the SR promoter system, which contains sequences from the SV40 early promoter and the long terminal repeat of human T cell leukemia virus type I (Takebe, Y. et al. (1988) Mol. Cell. Biol. 8:466-472).

In addition to the antibody chain genes and regulatory sequences, the expression vectors may carry additional sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216, 4,634,665 and 5,179,017, all by Axel et al.). For example, typically the selectable marker gene confers resistance to drugs, such as G418, hygromycin or methotrexate, on a host cell into which the vector has been introduced. Selectable marker genes include the dihydrofolate reductase (DHFR) gene (for use in dhfr-host cells with methotrexate selection/amplification) and the neo gene (for G418 selection).

For expression of the light and heavy chains, the expression vector(s) encoding the heavy and light chains is transfected into a host cell by any suitable techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is possible to express the antibodies of the disclosure in either prokaryotic or eukaryotic host cells, expression of antibodies in eukaryotic cells, and typically mammalian host cells, is most typical.

The present application further provides a host cell containing a nucleic acid molecule provided by the present application. The host cell can be virtually any cell for which expression vectors are available. It may be, for example, a higher eukaryotic host cell, such as a mammalian cell, a lower eukaryotic host cell, such as a yeast cell, and may be a prokaryotic cell, such as a bacterial cell. Methods of introducing a recombinant nucleic acid into a host cell are known in the art, including, for example, by calcium phosphate transfection, DEAE, dextran mediated transfection, electroporation or phage infection.

Suitable prokaryotic hosts for transformation include E. coli, Bacillus subtilis, Salmonella typhimurium and various species within the genera Pseudomonas, Streptomyces, and Staphylococcus.

Suitable eukaryotic hosts for transformation include yeast, insect (e.g., S2 cells), and mammalian cells. Mammalian host cells for expressing an anti-CTLA4 antibody of the disclosure include, for example, Chinese Hamster Ovary (CHO) cells (including dhfr-CHO cells, described in Urlaub and Chasin, Proc. Natl. Acad. Sci. USA 77:4216-4220 (1980); Sharp, J. Mol. Biol. 159:601-621 (1982)), NSO myeloma cells, COS cells, HEK293F cells, HEK293Tcells, and Sp2 cells. In particular, for use with NSO myeloma or CHO cells, another expression system is the GS (glutamine synthetase) gene expression system disclosed in WO 87/04462, WO 89/01036 and EP 338,841. In some embodiments, antibodies of the present application are produced in CHO cells. In some embodiments, antibodies of the present application are modified, and do not include a C-terminal lysine residue (e.g., the C-terminal lysine residue of an antibody heavy chain described herein is removed (such as before or during antibody production)). When expression vectors encoding antibody genes are introduced into mammalian host cells, the antibodies are produced by culturing the host cells for a period of time sufficient to allow for expression of the antibody in the host cells or secretion of the antibody into the culture medium in which the host cells are grown. Antibodies can be recovered from the culture medium using any suitable protein purification methods known in the art (e.g., protein A chromatography and/or ion exchange chromatography).

### V. Pharmaceutical Compositions, Kits, and Articles of Manufacture

In other aspects, the present application provides a composition comprising any one of the anti-CTLA4 antibodies described herein. In some embodiments, the composition is a pharmaceutical composition comprising the anti-CTLA4 antibody and a pharmaceutically acceptable carrier. The compositions can be prepared by conventional methods known in the art.

The term “pharmaceutically acceptable carrier” refers to any inactive substance that is suitable for use in a formulation for the delivery of an active agent (e.g., the anti-CTLA4 antibody). A carrier may be an anti-adherent, binder, coating, disintegrant, filler or diluent, preservative (such as antioxidant, antibacterial, or antifungal agent), sweetener, absorption delaying agent, wetting agent, emulsifying agent, buffer, and the like. Examples of suitable pharmaceutically acceptable carriers include water, ethanol, polyols (such as glycerol, propylene glycol, polyethylene glycol, and the like) dextrose, vegetable oils (such as olive oil), saline, buffer, buffered saline, and isotonic agents such as sugars, polyalcohols, sorbitol, and sodium chloride. The compositions may be in any suitable forms, such as liquid, semi-solid, and solid dosage forms. Examples of liquid dosage forms include solution (e.g., injectable and infusible solutions), microemulsion, liposome, dispersion, or suspension. Examples of solid dosage forms include tablet, pill, capsule, microcapsule, and powder. A particular form of the composition suitable for delivering an anti-CTLA4 antibody is a sterile liquid, such as a solution, suspension, or dispersion, for injection or infusion. Sterile solutions can be prepared by incorporating the antibody in the required amount in an appropriate carrier, followed by sterilization microfiltration. Generally, dispersions are prepared by incorporating the antibody into a sterile vehicle that contains a basic dispersion medium and other carriers. In the case of sterile powders for the preparation of sterile liquid, methods of preparation include vacuum drying and freeze-drying (lyophilization) to yield a powder of the active ingredient plus any additional desired ingredient from a previously sterile-filtered solution thereof. The various dosage forms of the compositions can be prepared by conventional techniques known in the art.

The relative amount of an anti-CTLA4 antibody included in the composition will vary depending upon a number of factors, such as the specific anti-CTLA4 antibody and carriers used, dosage form, and desired release and pharmacodynamic characteristics. The amount of an anti-CTLA4 antibody in a single dosage form will generally be that amount which produces a therapeutic effect, but may also be a lesser amount. Generally, this amount will range from about 0.01 percent to about 99 percent, from about 0.1 percent to about 70 percent, or from about 1 percent to about 30 percent relative to the total weight of the dosage form.

In addition to the anti-CTLA4 antibody, one or more additional therapeutic agents may be included in the composition. Examples of additional therapeutic agents are described herein in the “Methods of Treatment” section. The suitable amount of the additional therapeutic agent to be included in the composition can be readily selected by a person skilled in the art, and will vary depending on a number of factors, such as the particular agent and carriers used, dosage form, and desired release and pharmacodynamic characteristics. The amount of the additional therapeutic agent included in a single dosage form will generally be that amount of the agent, which produces a therapeutic effect, but may be a lesser amount as well.

In some embodiments, there is provided an article of manufacture comprising materials useful for the treatment of a cancer. The article of manufacture can comprise a container and a label or package insert on or associated with the container. Suitable containers include, for example, bottles, vials, syringes, etc. The containers may be formed from a variety of materials such as glass or plastic. Generally, the container holds a composition, which is effective for treating a cancer, described herein, and may have a sterile access port (for example, the container may be an intravenous solution bag or a vial having a stopper pierceable by a hypodermic injection needle). Package insert refers to instructions customarily included in commercial packages of therapeutic products that contain information about the indications, usage, dosage, administration, contraindications and/or warnings concerning the use of such therapeutic products. In some embodiments, the package insert indicates that the composition is used for treating a cancer. The label or package insert may further comprise instructions for administering the composition to a patient.

Additionally, the article of manufacture may further comprise a second container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Kits are also provided that are useful for various purposes, e.g., for treatment of a cancer described herein, optionally in combination with the articles of manufacture. Kits of the present application include one or more containers comprising any one of the compositions described herein (or unit dosage form and/or article of manufacture). In some embodiments, the kit further comprises other agents (e.g., one or more additional therapeutic agents) and/or instructions for use in accordance with any of the methods described herein. The kit may further comprise a description of selection of individuals suitable for treatment. Instructions supplied in the kits of the present application are typically written instructions on a label or package insert (e.g., a paper sheet included in the kit), but machine-readable instructions (e.g., instructions carried on a magnetic or optical storage disk) are also acceptable.

For example, in some embodiments, there is provided a kit comprising a pharmaceutical composition comprising any one of the anti-CTLA4 antibodies described herein and a pharmaceutically acceptable carrier; and instructions for administering the pharmaceutical composition to a subject having a cancer. In some embodiments, the kit further comprises a pharmaceutical composition comprising an additional therapeutic agent, such as a chemotherapeutic agent. In some embodiments, the kit comprises one or more assays or reagents thereof for determining a level of one or more biomarkers described herein (e.g., CD8+ T cells, CD4+ T cells, CD8+ Tₑₘ cells, CD4+ Tₑₘ cells, T_{reg} cells, a ratio of CD8+ Tₑₘ cells to T_{reg} cells, a ratio of CD4+ Tₑₘ cells to T_{reg} cells, NK cells, B cells).

The kits of the present application are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

The containers may be unit doses, bulk packages (e.g., multi-dose packages) or sub-unit doses. Kits may also include multiple unit doses of the pharmaceutical compositions and instructions for use and packaged in quantities sufficient for storage and use in pharmacies, for example, hospital pharmacies and compounding pharmacies.

### EXAMPLES

The invention can be further understood by reference to the following examples, which are provided by way of illustration and are not meant to be limiting.

### Example 1. Phase 1, Open-Label, Dose Escalation Study of TY21580 in Patients with Advanced/Metastatic Solid Tumors

The following example describes an ongoing phase 1 clinical trial to assess the safety and tolerability of TY21580, an anti-cytotoxic T-lymphocyte-associated protein 4 (CTLA4) fully human immunoglobulin G (IgG)1 monoclonal antibody (see ClinicalTrials.gov Identifier: NCT04501276).

TY21580. TY21580 is a fully human ligand-blocking anti-CTLA-4 mAb that target a conserved epitope with broad species cross-reactivity for translational fidelity. Without being bound by any theory or hypothesis, TY21580 was observed to have softer CTLA-4 ligand blocking and stronger ADCC for depleting regulatory T-cells than ipilimumab. In a head-to-head in vivo efficacy study, TY21580 was observed to have at least a five-fold greater preclinical antitumor activity in comparison with ipilimumab. In preclinical studies, TY21580 was well tolerated in rats and cynomolgus monkeys in four-week repeat-dose GLP toxicology studies at doses up to 30 mg/kg, and demonstrated an encouraging antitumor response in multiple immune-competent mouse tumor models in a dose-dependent manner both as a single agent (showing initial response at 0.02 mg/kg, and complete response at 0.1 mg/kg for tumor size of ˜100 mm³ in H22 liver cancer model) and in combination with other therapies. Without being bound by any theory or hypothesis, TY21580 balances safety and efficacy through a novel mechanism of action; TY21580 maintains its original physiological function via partial blocking of CTLA-4 ligand binding, and in conjunction, depletes T_{regs} in the tumor microenvironment via strong antibody-dependent cellular cytotoxicity (ADCC).

Objectives. The primary objectives of the study are to assess the safety and tolerability of TY21580 administered intravenously (IV) at escalating dose levels in adults with advanced/metastatic solid tumors, and to determine the maximum tolerated dose (MTD) and decide upon a recommended Phase 2 dose (RP2D) for TY21580. Secondary objectives of the study are to assess the pharmacokinetic (PK) profile of TY21580; to assess dose proportionality of key PK parameters (AUC, Cₘₐₓ); to assess the immunogenicity of TY21580; and to characterize the relationship between immunogenicity (anti-drug antibody (ADA) positivity) and PK, safety and efficacy parameters; to assess the preliminary antitumor activity of TY21580. An exploratory objective of the study is to assess pharmacodynamic biomarkers (including but not limited to cytokines (IL-1β, IL-2, IL-6, IL-10, interferon (IFN)-γ and tumor necrosis factor (TNF)-α), plasma proteins (sCTLA4, sPD-L1, sCD25, CXCL11), immune cells (tumor infiltrating immune (T_{reg}), CD8+ T_{EM}, Ki67), and tissue biomarkers (FoxP3, IFNγ, PD-L1) for TY21580.

Methodology. This is a Phase 1, open-label, dose escalation study in patients with advanced/metastatic solid tumors. The anticipated number of patients in Phase 1 is up to approximately 60 patients and is not driven by statistical considerations. The actual total number of patients is determined by the composite elements of this protocol, the dose escalation and dose level expansion criteria, the observed dose-limiting toxicities (DLTs)—present or absent, any objective evidence of antitumor activity, and other clinical safety data that is collectively taken together. Safety oversight is provided by a Safety Review Committee (SRC) comprised of the Principal Investigator (PI), the medical monitors and the Sponsor.

All potential study candidates provide informed consent and undergo Screening procedures before participating in the study. After a Screening period of up to 28 days, qualified patients are enrolled to receive their assigned dose regimen of TY21580. TY21580 is administered intravenously (IV) over 60-90 minutes with planned doses every 3 weeks (Q3W) for the first 4 cycles. If no intolerable toxicities occur during the first consecutive 4 treatment cycles, administration of TY21580 may continue every 12 weeks (Q12W) for a total duration of up to 2 years.

A treatment cycle is 21 days with one IV dose of TY21580 administered on Day 1. DLTs are evaluated during the initial 21 days. Toxicities are assessed using the National Cancer Institute Common Terminology Criteria for Adverse Events (NCI CTCAE) v5.0. Patients are treated on study with TY21580 until documented confirmed disease progression according to RECIST v1.1 and/or iRECIST, development of significant toxicity, withdrawal of consent, or other discontinuation/withdrawal reason; whichever occurs first. During the study, patients are evaluated for safety and toxicity, PK, immunogenicity, objective response, DOR, PFS, OS and pre-specified biomarkers per protocol.

The study utilizes an Accelerated Titration Design (ATD) in the lower dose levels (DL1 and DL2) followed by a traditional 3+3 dose escalation design in the higher dose levels until the RP2D is determined. The starting dose is DLL. The study is conducted using nine potential dose levels administered by IV infusion as shown in Table 1, below.

TABLE 1
Potential TY21580 Dose Levels

| | Dose Level | | Percent Increase |
|---|---|---|---|
| Design | (DL)* | Dose, mg/kg | from Previous Dose |
| Accelerated titration | DL-1 | 0.001 | — |
| design | DL1 | 0.003 | 300% |
| | DL2 | 0.01 | 333% |
| Traditional 3 + 3 | DL3 | 0.03 | 300% |
| design** | DL4 | 0.1 | 333% |
| | DL5 | 0.3 | 300% |
| | DL6 | 1.0 | 333% |
| | DL7 | 3.0 | 300% |
| | DL8 | 6.0 | 200% |
| | DL9 | 10.0 | 166% |

*The proposed doses, schedule(s), and PK time points may be reconsidered and amended during the study based on safety data and observed systemic exposures and as determined by the SRC. DL-1 is a lower titration dose to be used in the event of DLTs or clinically significant grade ≥2 toxicity AEs in DL1.
**If a single patient experiences a DLT or two ≥Grade 2 drug-related toxicities during the 21-day DLT evaluation period, traditional 3 + 3 dose escalation criteria will apply for that dose level with enrollment of additional patients. All subsequent dose levels will then follow traditional 3 + 3 dose escalation criteria.

During the ATD phase, 1 patient per dose level is treated. If the patient experiences a DLT or two ≥Grade 2 drug-related toxicity, the dose level is expanded according to a 3+3 design. From DL3 (0.03 mg/kg) onwards, dose escalation follows a traditional 3+3 design with 3 or 6 patients treated at each dose level, depending upon the incidence of DLTs. Initially, 3 patients are enrolled into the dose level with the sentinel patient treated at least 24 hours before the subsequent patients.

From DL3 (0.03 mg/kg) onwards, dose escalation will follow a traditional 3+3 design with 3 or 6 patients treated at each dose level, depending upon the incidence of DLTs. Initially, 3 patients will be enrolled into the dose level with the sentinel patient treated at least 24 hours before the subsequent patients. Dose escalation decisions will be made by the SRC. Dose escalation rules for traditional 3+3 design are as follows in Table 2, below.

TABLE 2
Dose Escalation Rules for Traditional 3 + 3 Design

| Number of | |
|---|---|
| Patients | |
| with DLT at | |
| a Given | |
| Dose Level | Dose Escalation Decision Rule |
| 0 of 3 | Enroll 3 patients at next dose level (next/higher dose |
| | level/dose escalation) |
| 1 of 3 | Enroll 3 additional patients at current dose level (current |
| | dose) |
| 2 of 3 | Stop enrollment. Enroll 3 more patients at the previous dose |
| | level (previous/lower level/dose de-escalation) if only 3 |
| | were previously enrolled, or at an intermediate dose level |
| 1 of 6 | Enroll 3 patients at next dose level (next/higher dose |
| | level/dose escalation) |
| ≥2 of 6 | Stop enrollment. If at DL-1 or DL1, the study will be |
| | stopped. If at DL2 or above, enroll 3 more patients at the |
| | previous dose level (previous/lower dose level/dose de- |
| | escalation) if only 3 were previously enrolled, or at an |
| | intermediate dose level |

The highest potential dose level in this study will not exceed 10 mg/kg as the predetermined maximum administered dose (MAD). Once either the MTD or MAD are reached, the RP2D is determined, including selection and treatment of an intermediate dose level between the pre-specified dose levels. The RP2D is decided upon based primarily on the observation of either the MTD, or by the MAD in the absence of an MTD. Options for consideration for the RP2D will also include dose levels below the MTD or MAD, as well as intermediate doses between the pre-specified dose levels, based on an overall assessment of all safety data, as well as all available PK and pharmacodynamic data, and documented objective response observations during dose escalation. Additional cohorts may be added as expansion cohorts to further evaluate the RP2D.

Intra-patient dose escalation up to the dose level cleared by the SRC is permitted for a patient who has completed at least two cycles of treatment at the originally enrolled dose level.

The treatment may proceed beyond progressive disease by RECIST v1.1 if there is evidence of clinical benefit without worsened tumor-related symptoms and/or unacceptable toxicity. All progressive disease should be confirmed as per iRECIST: a repeat scan will be required, preferably after 4 weeks and no later than 6 weeks. Once disease progression is confirmed as per and/or iRECIST, ongoing treatment is not be allowed. Otherwise, the study treatment may continue for a total duration of up to 2 years, or until disease progression, unacceptable toxicity, or withdrawal of consent; whichever occurs first.

All patients, except those who have withdrawn consents, are followed up for safety follow-up assessments, at 30-day and 90-day from the date of the last dose, as well as for survival, every 12 weeks from the date of the last dose. Patients who discontinue treatment due to intolerable adverse events related to TY21580 are followed up until the adverse events have returned to Grade 0 or 1, or become stable, or until the patient receives new non-protocol treatment. Survival follow-up continues until receipt of another antitumor therapy, patient being lost to follow-up or death, whichever occurs first. The Sponsor may also decide to terminate the study at any time.

Criteria for Eligibility. Patients must meet all of the following inclusion criteria to be eligible for participation in this study:
1) ≥18 years of age at the time of informed consent.
2) Eastern Cooperative Oncology Group (ECOG) performance status 0 or 1 with no deterioration over the previous 2 weeks.
3) Patients with advanced or metastatic solid tumors, confirmed by histopathology, who have progressed after all standard therapies, or for whom no further standard therapy exists. Patients who have declined standard therapy or have no access to standard therapy may be enrolled and the reasons for lack of access need to be documented.
4) Patients who are refractory or relapsed to prior anti-CTLA4 checkpoint inhibitors or anti-Programmed cell death protein 1 (PD-1) will also be recruited if they meet all eligibility criteria.
5) Adequate hematologic function, defined by the following:
   a. Absolute neutrophil count (ANC)≥1.5×10⁹/L, without the use of granulocyte colony stimulating factor such as filgrastim within 2 weeks prior to study treatment.
   b. Platelet count ≥100×10⁹/L without transfusion within 2 weeks (≤14 days) prior to study treatment. Patients with hepatocellular carcinoma and a platelet count ≥75×10⁹/L.
   c. Hemoglobin ≥9 g/dL without transfusion or erythropoietin within 2 weeks (≤14 days) prior to study treatment.
6) Aspartate transaminase (AST) and alanine aminotransferase (ALT)≤3×upper limit of normal (ULN), and total bilirubin ≤1.5×ULN. Exception: Patients who have serum bilirubin increases due to documented underlying Gilbert's Syndrome or familial benign unconjugated hyperbilirubinemia. Patients with hepatocellular carcinoma and hepatic metastases may have AST and ALT up to ≤5×ULN.
7) Adequate renal function defined by either a creatinine clearance ≥60 mL/min (by Cockcroft-Gault formula) or serum creatinine (SCr)<1.5×ULN
8) Coagulation tests, defined by the following:
   a. Activated partial thromboplastin time (aPTT)≤1.5×ULN.
   b. International normalized ratio (INR)≤1.5×ULN. Exception: INR 2 to ≤3×ULN is acceptable for patients on Warfarin anticoagulation.
9) Left ventricular ejection fraction (LVEF)≥50% measured by multiple-gated acquisition (MUGA) or echocardiogram (ECHO).
10) Previous antitumor therapy (including endocrine, chemoradiotherapy/radiotherapy, targeted therapy, or immunotherapy) that has ended at least 4 weeks prior to administration of TY21580. Patients who failed prior anti-CTLA4 checkpoint inhibitors may be considered eligible if they meet all eligibility criteria.
11) Previous adverse events have been improved to baseline or ≤Grade 1 NCI CTCAE v5.0 (except for patients with alopecia).

Exclusion criteria include any one of the following:
1) Pregnant or breastfeeding females.
2) Females of childbearing potential and males whose partners are of childbearing potential who do not agree to the use of two forms of highly effective contraception during the treatment period and for 120 days after the last dose of study drug.
3) Treatment with any investigational drug within 4 weeks prior to the first dose of study drug.
4) Grade ≥3 immune-related AEs (irAEs) or irAE that lead to discontinuation of prior immunotherapy.
5) Untreated or uncontrolled central nervous system (CNS) tumors or metastases with following exceptions:
   a. Clinically stable Magnetic Resonance Imaging (MRI) scans (at least two consecutive scans within 6 months) and no progressive or uncontrolled neurologic symptoms or signs (e.g., seizures, headaches, central nausea/emesis, progressive neurologic deficits) for at least 4 weeks.
   b. Any untreated asymptomatic brain metastases not requiring immediate local or systemic therapy.
6) History of life-threatening hypersensitivity or known to be allergic to protein drugs or recombinant proteins or any ingredients contained in the TY21580 drug formulation
7) Patients with active autoimmune disease or a documented medical history of autoimmune disease or symptoms that required systemic use of pharmacologic dose of corticosteroid and/or immunosuppressant. Exceptions are patients with vitiligo, resolved childhood asthma/atopy, and type I diabetes mellitus or hypothyroidism that can be managed by replacement therapy.
8) Patients requiring systemic treatment with corticosteroids (>15 mg/day prednisone or equivalent) or other immunosuppressive medications within 21 days before the planned first dose of study drug. Inhaled or topical steroids, and adrenal replacement steroid doses ≤15 mg daily prednisone equivalent are permitted in the absence of active autoimmune disease. Ophthalmologic, nasal, inhaled and intra-articular injections of steroids are allowed.
9) Peripheral neuropathy ≥Grade 2.
10) Patients receiving granulocyte colony stimulating factor (G-CSF), granulocyte macrophage colony stimulating factor (GM-CSF), erythropoietin, or blood (red blood cell [RBC] or platelet) transfusion within 14 days prior to the first dose of the study drug.
11) Any evidence of underlying liver dysfunction due to other causes; Any history of significant alcohol abuse, alcoholic or drug-induced hepatitis, or documented nonalcoholic steatohepatitis
12) Active viral (any etiology) hepatitis patient are excluded. Hepatitis B virus (HBV) carriers are ineligible. Cured Hepatitis C (HCV) (negative HCV ribonucleic acid [RNA] test) patients may be enrolled after consulting with the medical monitor.
13) Any uncontrolled active infections requiring systemic antimicrobial treatment (viral, bacterial, or other), or uncontrolled or poorly controlled diabetes as evidenced by Screening (baseline) HgbA-1c≥7.5, asthma, chronic obstructive pulmonary disease (COPD), or other conditions that pose a risk to the patient participating on study.
14) Known human immunodeficiency virus (HIV) positive status.
15) Patients with any type of primary immunodeficiency or autoimmune disorder requiring treatment.
16) Major surgery within 4 weeks prior to the first dose of the study drug.
17) Prior organ allograft transplantations or allogeneic peripheral blood stem cell (PBSC)/bone marrow (BM) transplantation.
18) Clinically significant cardiac conditions, including myocardial infarction within the last six months, uncontrolled angina, viral myocarditis, pericarditis, cerebrovascular accident, or other acute uncontrolled heart disease <3 months prior to the first dose of the study drug; LVEF<50%, New York Heart Association (NYHA) Class III or IV congestive heart failure, or uncontrolled hypertension.
19) Pulmonary embolism or deep vein thrombosis within 3 months prior to the first dose of study drug.
20) Live viral vaccine therapies within 4 weeks prior to the first dose of study drug.
21) Any known, documented, or suspected history of illicit substance abuse.
22) Any other disease or clinically significant abnormality in laboratory parameters, including serious medical or psychiatric illness/condition, which in the judgment of the Investigator might compromise the safety of the patient or integrity of the study, interfere with the patient participation in the trial or compromise the trial objectives.

Safety Evaluations. Safety assessments are carried out during specified periodic physical examination findings, vital signs, ECOG performance status, laboratory variables (e.g., liver tests/monitoring, hematology, coagulation tests, serum chemistry, urine tests and pregnancy test), ECG, and AEs. AEs are graded according to the NCI CTCAE v5.0. Investigators and site personnel are responsible for properly documenting and reporting AEs/SAEs. Prior to dose escalation, the SRC reviews the safety data from the current level after all patients have completed the first cycle of 21 days, to determine whether escalating (or de-escalating) to next dose level should proceed.

Efficacy Evaluations. Tumor assessments for response/progression are performed at baseline, and every 6 weeks (±1 week) for the first 4 cycles. If treatment continues beyond 4 cycles, then assessments are carried out every 9 weeks (±1 week) for the remaining treatment duration thereafter, until disease progression or death, treatment/study discontinuation due to treatment toxicity, loss to follow-up, withdrawal of consent, start of new cancer treatment, or study completion/closure, whichever occurs first. The exploratory evaluation of efficacy is based on tumor assessment by the Investigator per RECIST v1.1 and/or iRECIST.

Pharmacokinetic and Immunogenicity Evaluations. Blood samples are collected from all patients during the first cycle to determine the serum concentration of TY21580. Pharmacokinetic (PK) parameters are monitored more intensively during the first treatment cycle. Starting from the second administration, plasma concentration of TY21580 is collected within 30 minutes before the administration of the drug (C_{trough}) and at the end of infusion. The PK sampling time points may be adjusted based on cumulative data. Non-compartmental analysis will be conducted using WinNonlin 6.4 or higher version. PK parameters include, but not limited to AUC_{0-21d}, AUCₗₐₛₜ, Cₘₐₓ, Tₘₐₓ, t_{1/2}, MRT, CL, V_{d} are reported. Dose proportionality is also assessed for AUC and Cₘₐₓ.

Blood samples for ADAs are collected at pre-dose of cycles 1, 2, 3, 4, and every two cycles thereafter if treatment will continue beyond 4 cycles. Additionally, ADA samples are collected at the end of the treatment and at the 30- and day 90-day follow-up visits after the last dose. Neutralization activity is evaluated if ADA is positive.

### Results

Interim results of the study are provided in the drawings and are described below.

### General Observations

To date, TY21580 has been well tolerated in more than 10 patients in dose escalation up to 0.3 mg/kg, and no dose-limiting toxicity (DLT), or unexpected safety signals including treatment-related serious adverse events (SAEs, i.e., drug related G3 and G4 toxicities), colitis or hepatitis have been observed so far. The terminal half-life of TY21580 was within the normal range of an IgG1 based antibody, which is around 14 days with good exposure.

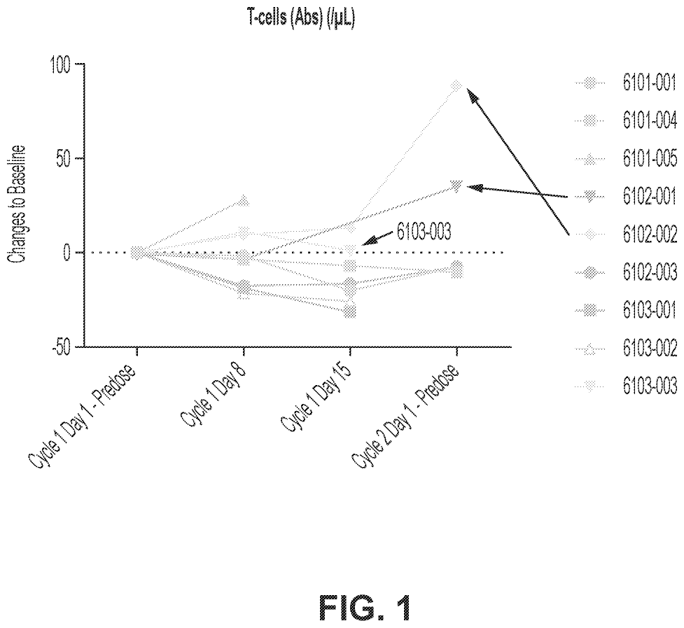

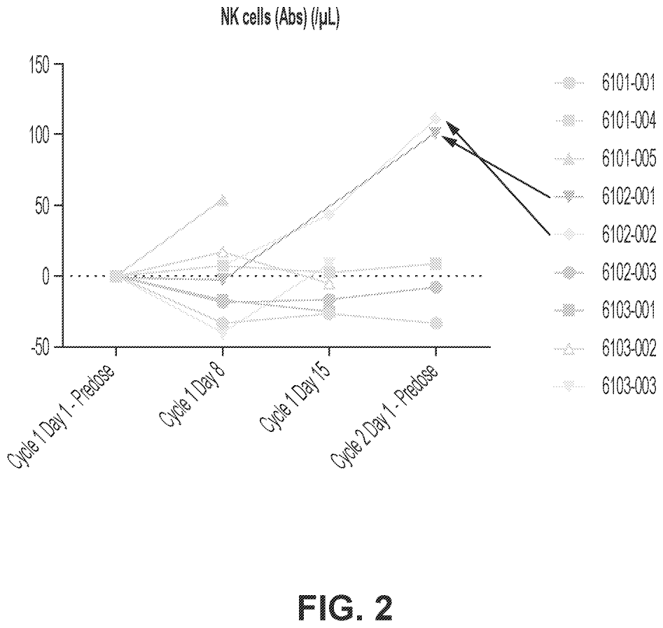

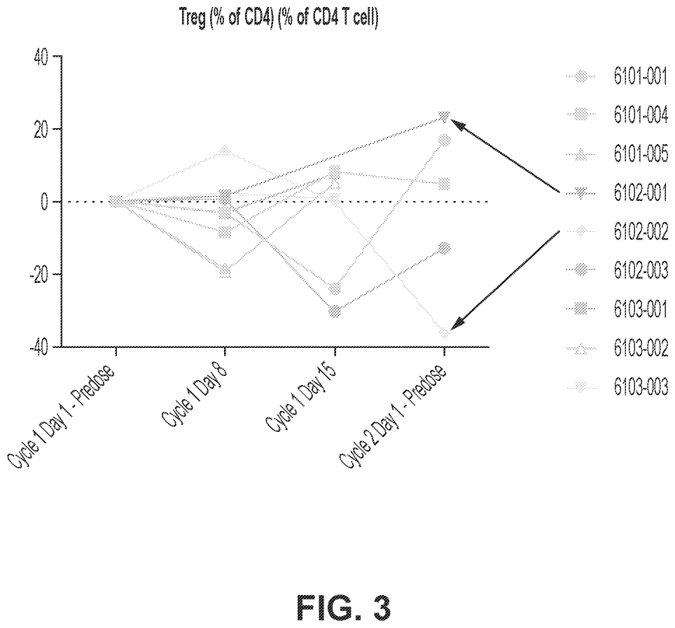

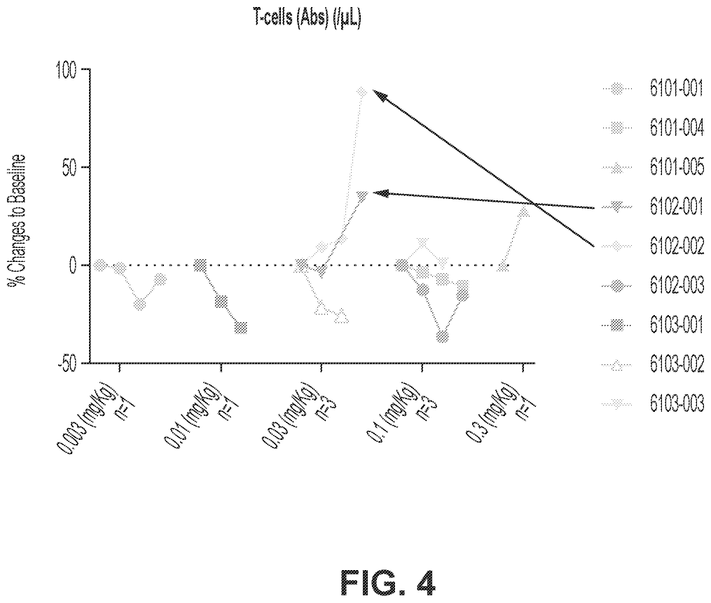

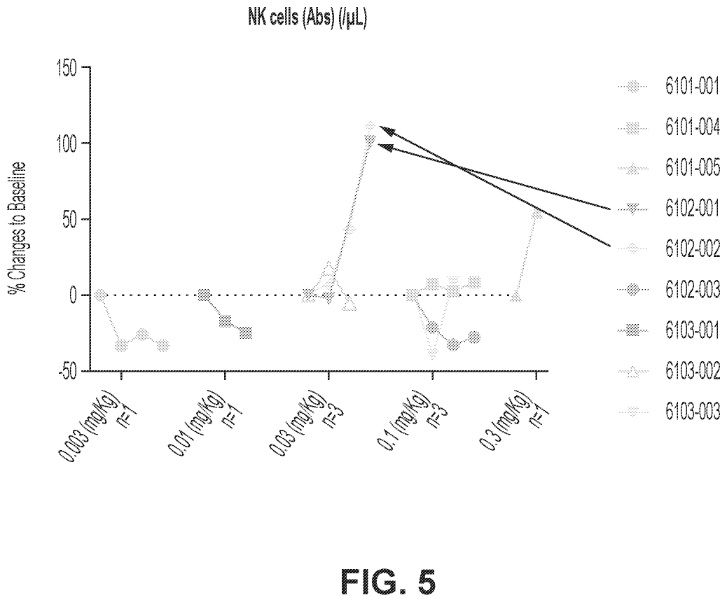

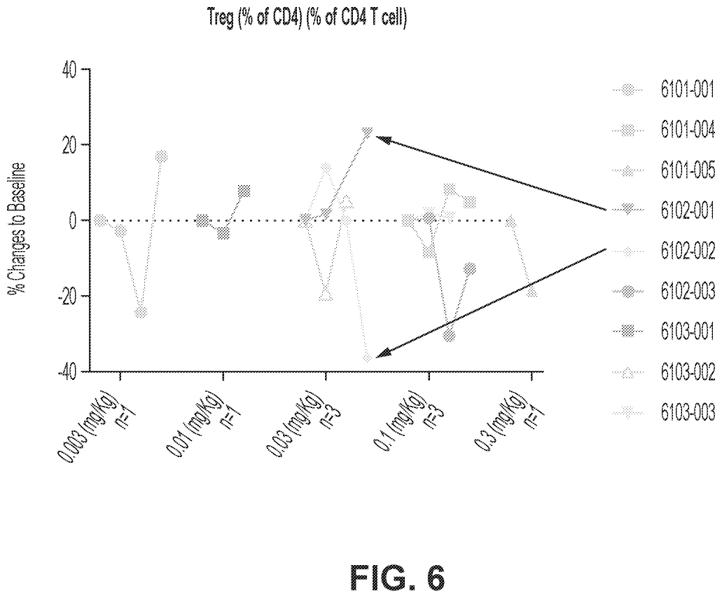

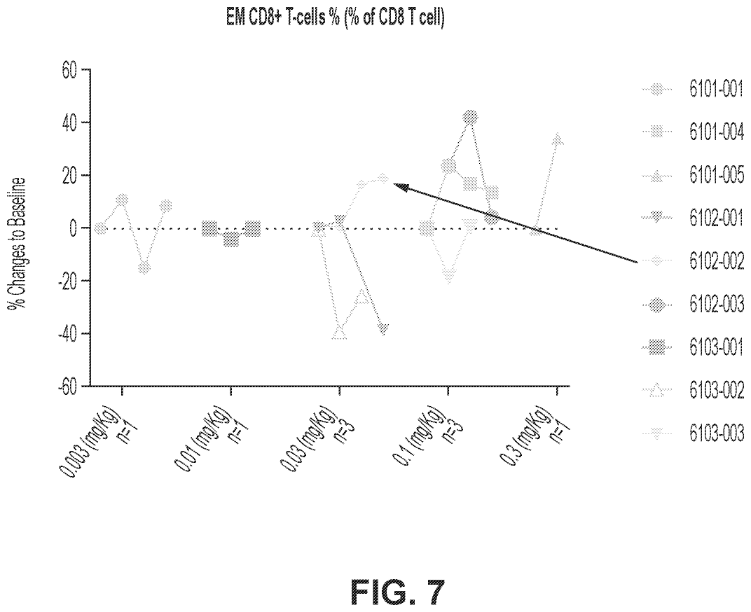

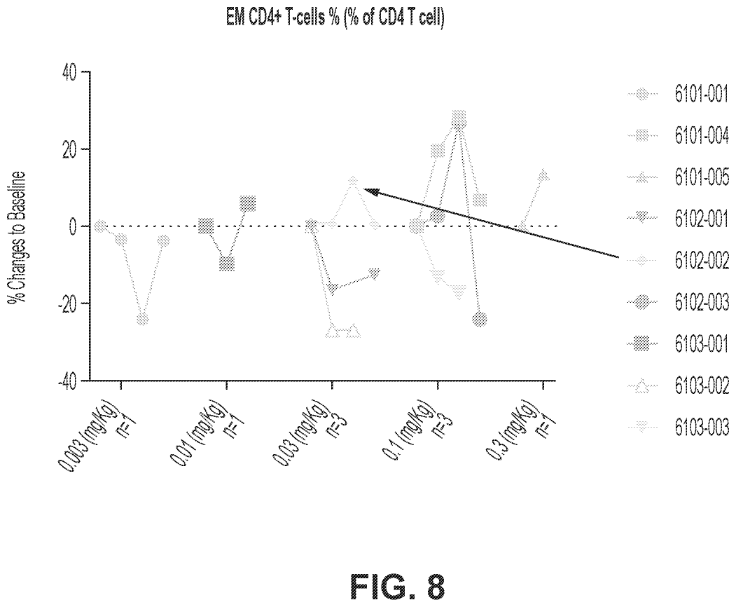

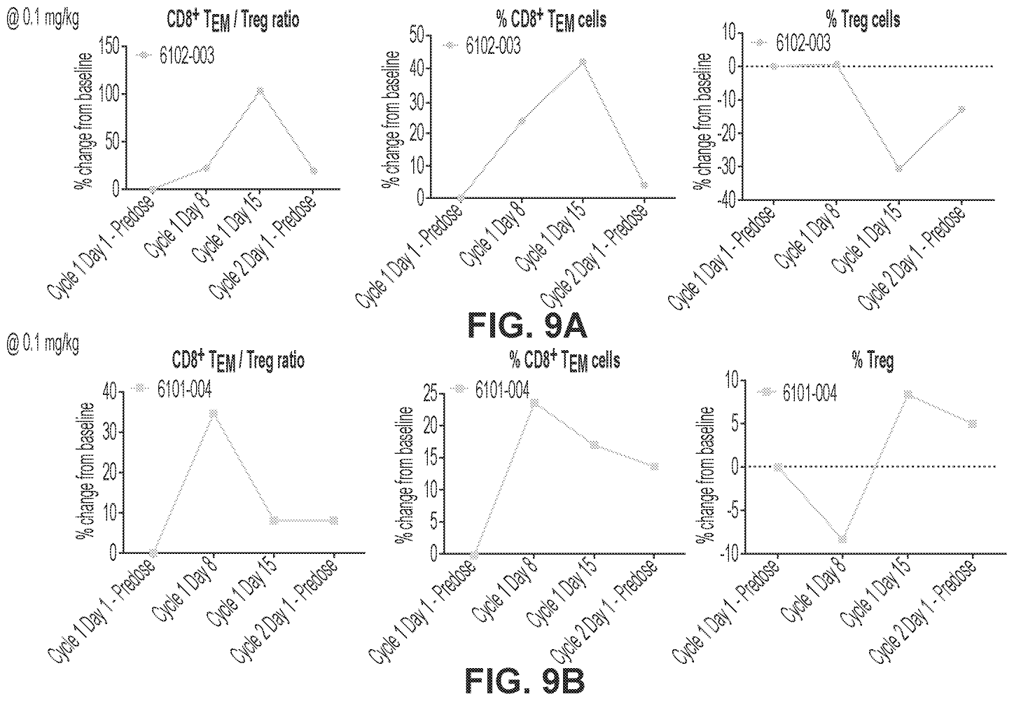

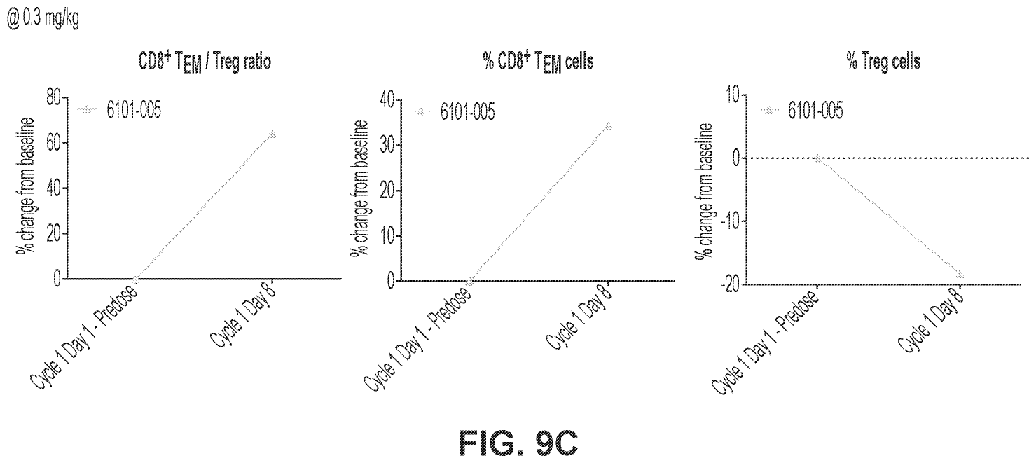

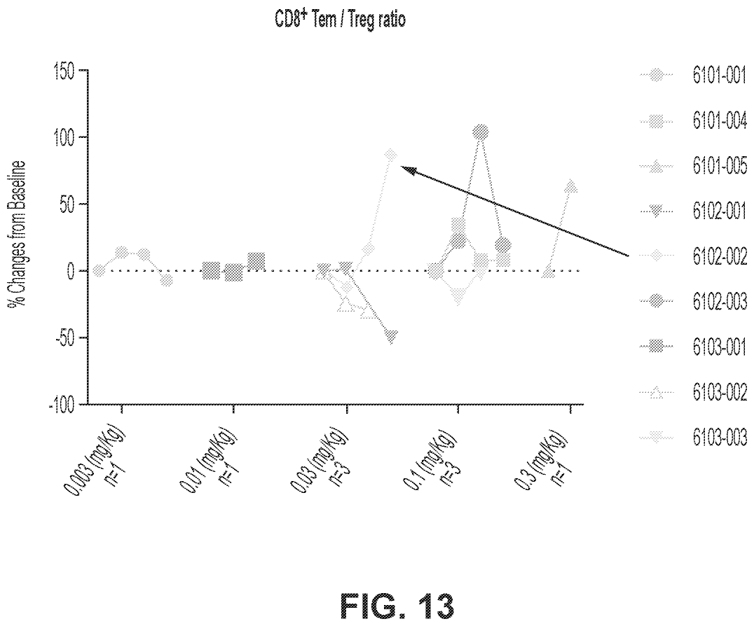

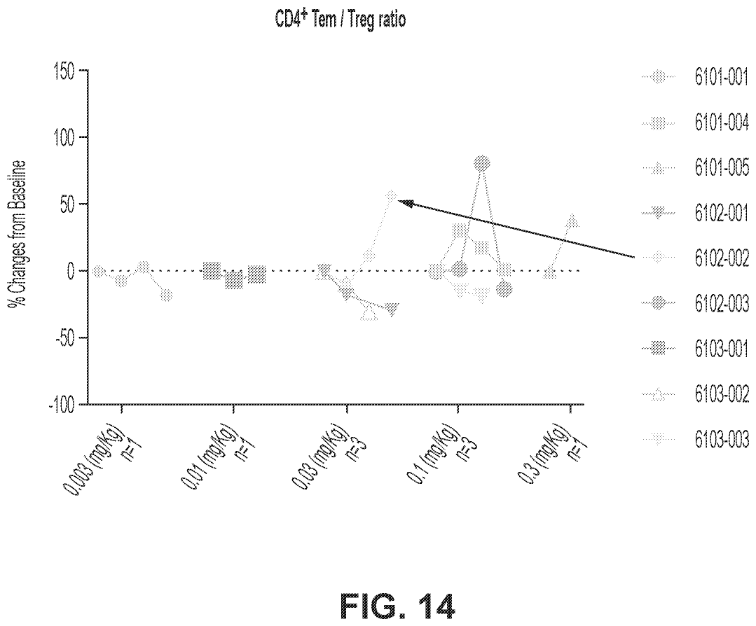

Interim pharmacodynamics (PD) results are presented in FIGS. 1-9C, and FIGS. 13-14. Specifically, TY21580 induced an immune cell response for T cells at dose levels from 0.03 to 0.3 mg/kg doses (FIGS. 1, 4). An effective CD8 and CD4 T cell increase was observed first, followed by a decrease in a dose-dependent way from 0.03, 0.1 and 0.3 mg/kg doses, similar to treatment with anti-PD1 and CD137 antibodies, etc. For certain patients, there was a decrease in the percentage of T_{reg} cells at 0.03. 0.1 and 0.3 mg/kg doses (FIG. 6), and a significant increase in the percentage of CD8+ effector memory T cells (FIG. 7) and CD4+ effector memory T cell (FIG. 8) at 0.03. 0.1 and 0.3 mg/kg doses. Additionally, for these patients, the ratio of CD8+ effector memory T cell/regulatory T cell (T_{EM}/T_{reg}) (FIG. 13) and the ratio of CD4+ effector memory T cell/regulatory T cell (T_{EM}/T_{reg}) (FIG. 14) showed significant increase. There was an increase in the level of NK and B cells for certain patients (FIGS. 2, 5).

FIGS. 4-6 and 13-14 show the relationship between TY21580 dose and immune cell response. A dose-dependent change in CD8+ T_{EM}/T_{reg} ratio, an important pharmacodynamic (PD) biomarker indicating immune activation, was observed for patients dosed by TY21580 from 0.003 to 0.3 mg/kg (FIG. 13). Similarly, as shown in FIG. 14, there was a dose-dependent change in the CD4⁺ T_{EM}/T_{reg} ratio. A strong trend in dose-dependent CD8+ T_{EM}/T_{reg} ratio and CD4+T_{EM}/T_{reg} ratio upon TY21580 treatment at doses of 0.003 to 0.3 mg/kg demonstrates that TY21580 can stimulate immune cell effectively at a dose level as low as 0.03 mg/kg. This dose-dependent change in CD8+ T_{EM}/T_{reg} ratio, is a strong clinical proof of mechanism (POM) for TY21580. These results are consistent with preclinical observations that TY21580 would be potent in sensitive tumor model such as H22 at dose as low as 0.02 mg/kg to induce tumor response, and achieve a complete response (CR) at 0.1 mg/kg. In particular, a steady increase in immune cells including CD8+ T_{EM}/T_{reg} ratio was observed for a patient (subject 6103-002) who has received 3 cycles of TY21580 at 0.03 mg/kg after this patient became refractory and resistant to prior pembrolizumab therapy for >25 cycles. The patient developed irAE pruritus. Additionally, subject 6103-003 also developed an irAE Grade 1 rash, at end of treatment on day 22. This Grade 1 treatment-related immune-mediated pruritus was consistent with the mechanism of action by TY21580 treatment.

Monitoring is ongoing to confirm the dose-dependent response of the various biomarkers to TY21580 treatment.

Upon treatment with TY21580, significant increases in absolute T cell and N cell levels, and decreases in the percentage of T_{reg} cells correlates with favorable clinical outcomes. The Phase 1 dose escalation data demonstrates the clinical proof of mechanism for TY21580 in targeting CTLA-4, consistent with preclinical observations for the potency of TY21580 starting from 0.03 to 0.3 mg/kg. It is anticipated that TY21580 has the potential to overcome the limitations of existing anti-CTLA-4 checkpoint inhibitors on the market, and extend the market potential beyond current anti-CTLA-4 inhibitors in both monotherapy and combination settings. The anti-CTLA-4 therapeutics described herein may improve the clinical benefits by expanding clonal diversity, infiltrating into cold tumors, and treating patients resistant/refractory to current immuno-therapies.

### Subject 6102-002

Subject 6102-002 was enrolled in the study. Subject 6102-002 was a white male, 60 years of age, with an urothelial carcinoma (kidney), and an ECOG Scale of Performance Status of 0. Subject 6102-002 had previously received 25 cycles of Pembrolizumab plus Epacadostat from Apr. 15, 2018 to Sep. 19, 2019. The subject had showed an initial partial response and then had become refractory and resistant to Pembrolizumab therapy for >25 cycles. Three 0.03 mg/kg doses (2.7+ mg) of TY21580 were administered to subject 6102-002, and the dose was escalated to 0.1 mg/kg on Feb. 24, 2021. Subject 6102-002 will be escalated to higher dose once cleared. Subject 6102-002 developed the immune-related adverse event (irAE) pruritus. This grade 1 drug related immune toxicity at 0.03 mg/kg suggested target modulation at 0.03 mg/kg at an estimated 60% target engagement, and is consistent with the mechanism of action by TY21580 treatment. A summary of the treatment emergent adverse events experienced by subject 6102-002 is provided in Table 3, below.

TABLE 3
Summary of treatment emergent adverse
events for subject 6102-002

| Treatment | | | | | |
|---|---|---|---|---|---|
| Emergent | | | | | |
| Adverse | | | | | |
| Events | | | | Cau- | Out- |
| (TEAEs) | Start | Stop | Grade | sality | come |
| Headache | 2020 Dec. 22 | 2020 Dec. 22 | 1 | Not | Rec-ed |
| | (Day 1) | (Day 1) | | related | |
| Myalgia | 2021 Jan. 5 | 2021 Jan. 13 | 1 | Not | Rec-ed |
| | (Day 15) | (Day 23) | | related | |
| Pruritus/ | 2021 Jan. 4 | Ongoing | | Related | Rec-ing |
| irAE | (Day 14) | | | | |
| Gastro | 2021 Jan. 14 | | 1 | Not | Rec-ing |
| reflux | (Day 24) | | | related | |

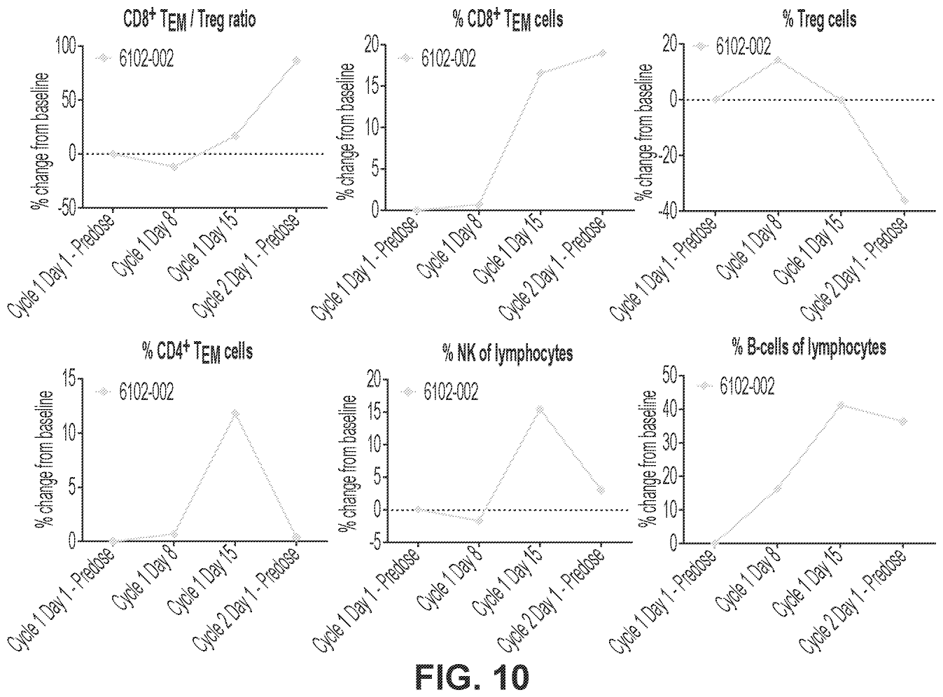

FIG. 10 provides lymphocyte profiling results of subject 6102-002 overtime. As shown in FIG. 10, in subject 6102-002, there was an effective CD8 T cell increase continuously, but CD4 T cells increased first and then decreased at 0.03 mg/kg. In subject 6102-002, T_{reg} cells decreased at 0.03 mg/kg. The CD8+ T_{EM}/T_{reg} ratio showed a significant increase. Further, NK and B cells increased. An analysis of immune cell subpopulation by flow cytometry revealed stimulation of T cells, NK and B cell proliferation, and T_{reg} depletion upon TY21580 treatment, especially for the big increase in CD8⁺ T_{EM}/T_{reg} ratio. This trend seemed to be followed by patients dosed at 0.1 and 0.3 mg/kg (as described above). PD results for subject 6102-002 are also shown in FIGS. 1-8.

### Population Pharmacokinetic (PK) Modeling

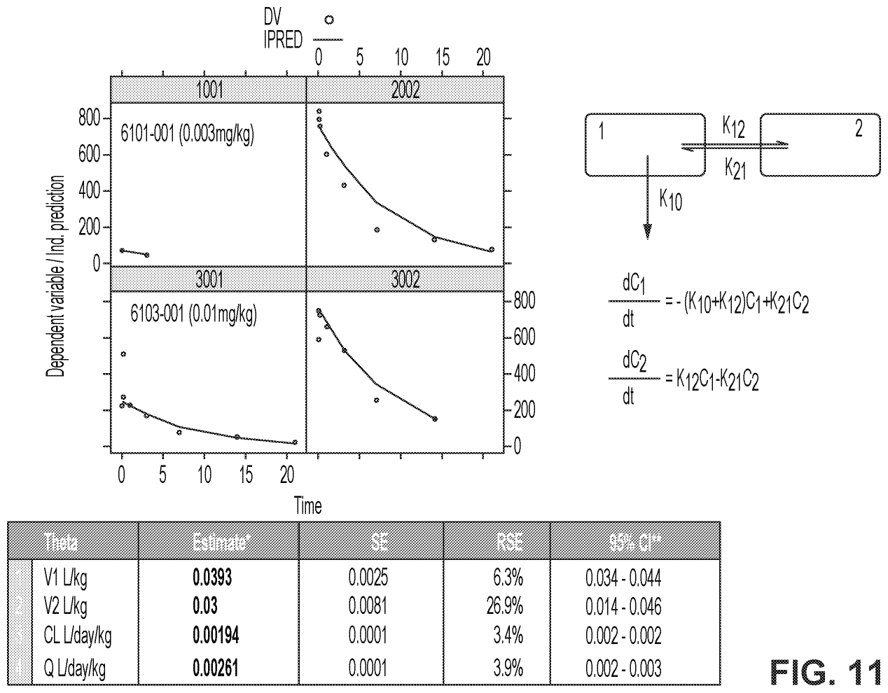

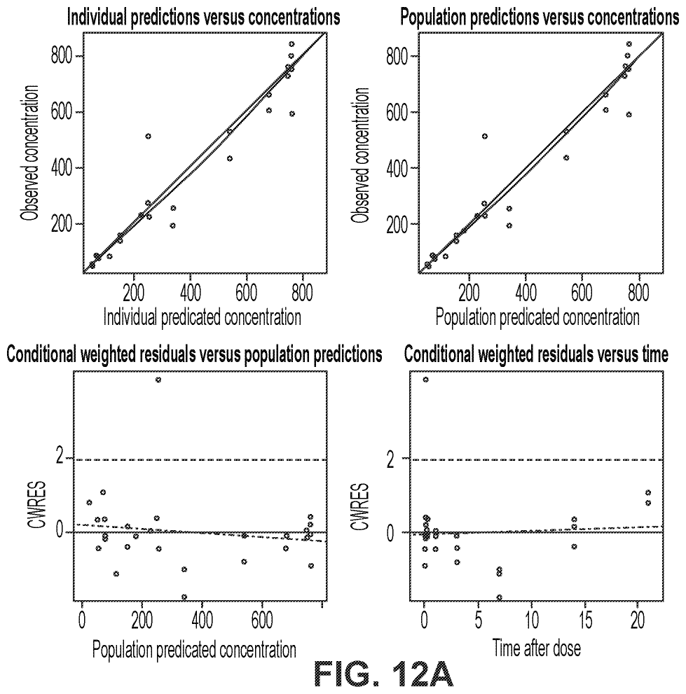

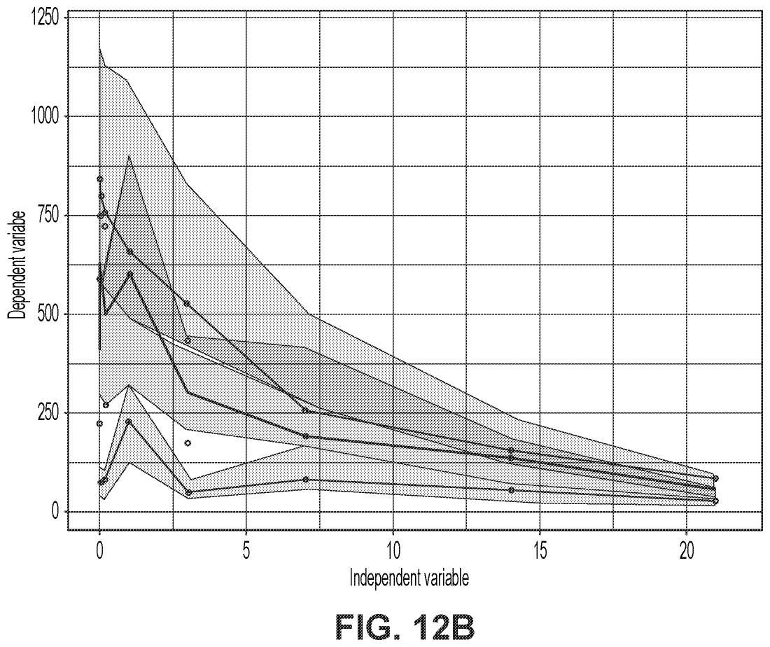

Preliminary PK population modeling was performed for the study. A 2-compartment linear PK model described the current PK data reasonably well across the studied doses (FIG. 11). All PK parameters were estimated with good precision, except for V2 (peripheral volume of distribution). The model-estimated population terminal half-life was ˜14 days. The model-estimated population central volume of distribution (VI) was similar to plasma volume (e.g., 40 mL/kg). Goodness-of-fit plots were generated (FIGS. 12A-12B). The fit of the model was reasonably good based on the diagnostic plots. Additional emerging PK data is used to refine the parameter estimates in real-time.

A second interim analysis of the ongoing study are provided in the drawings and are described below.

### Clinical Summary

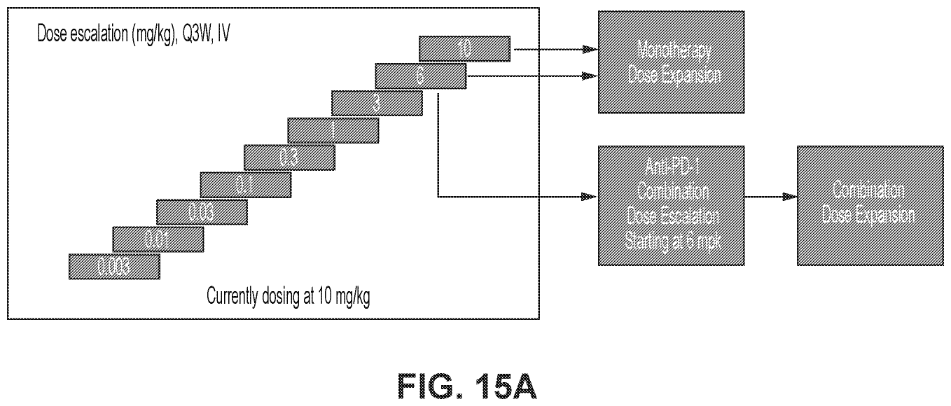

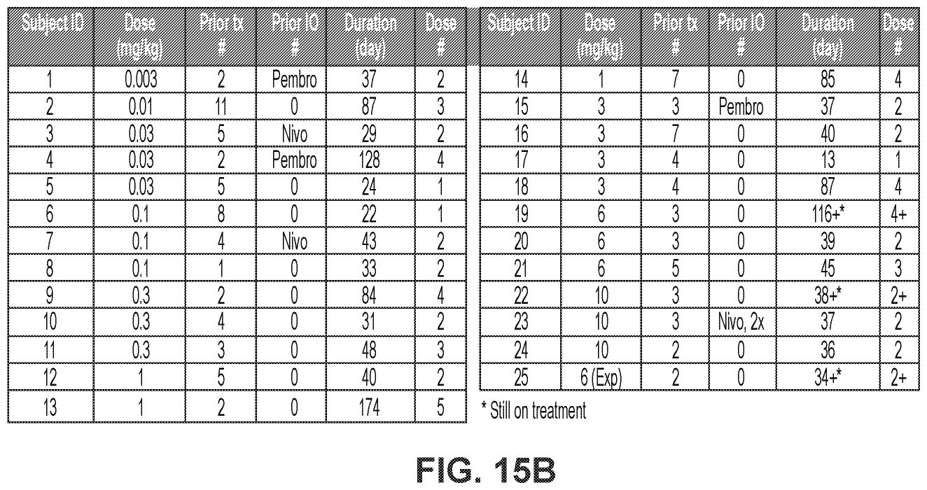

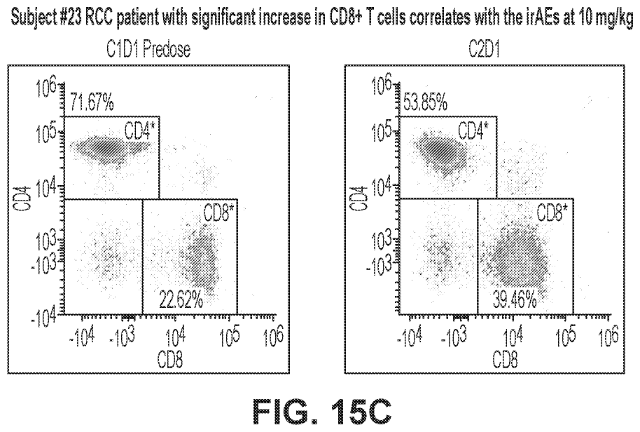

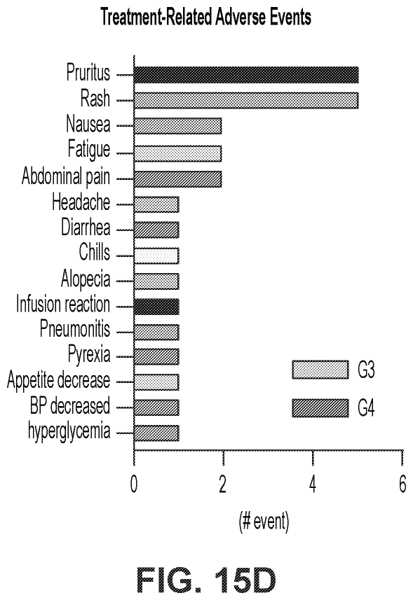

To date, 25 patients with advanced/metastatic solid tumors were treated with TY21580 at nine dose escalation cohorts (i.e., 0.003 mg/kg, 0.01 mg/kg, 0.03 mg/kg, 0.3 mg/kg, 1.0 mg/kg, 3.0 mg/kg, 6.0 mg/kg, and 10 mg/kg, n=24 patients) and 6 mg/kg expansion (n=1). These patients have advanced metastatic disease and have been treated across 15 different tumor types, 68% of which are IO insensitive tumors. (FIG. 15B). TY21580 has been well tolerated up to 10 mg/kg, once every three weeks (Q3W), with no DLT and no dose-dependent toxicities identified (FIG. 15D). Currently, patients are enrolling at the next higher dose level, 20 mg/kg, to continue dose escalation following the amended protocol and 6.0 mg/kg dose expansion.

### Pharmacokinetics

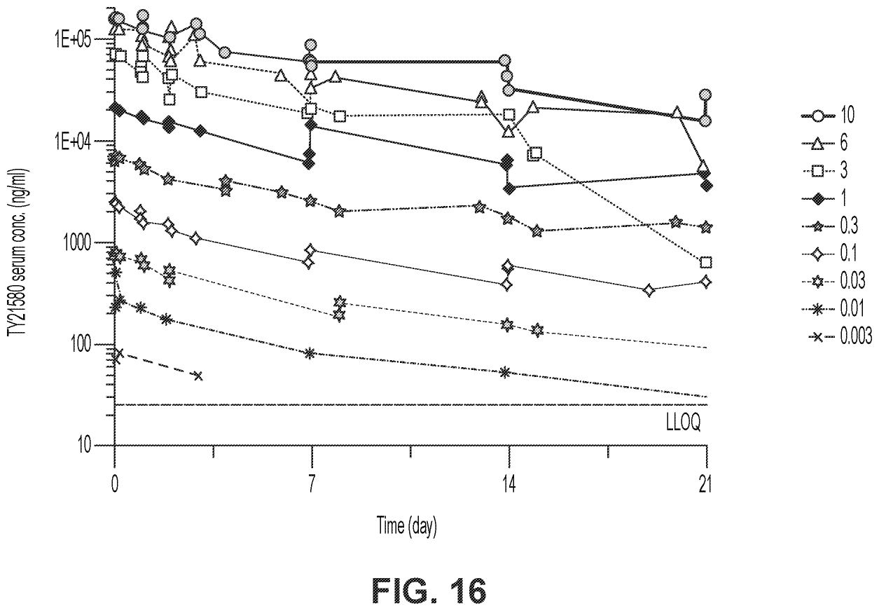

TY21580 shows a dose-proportional increase in drug exposure with a half-life of approximately 10 days (estimated at about 10±5 days up to 10 mg/kg). FIG. 16 shows the serum pharmacokinetics of TY21580 after IV infusion, as assessed by non-compartmental analysis and population PK modeling approaches.

### Pharmacodynamics Results

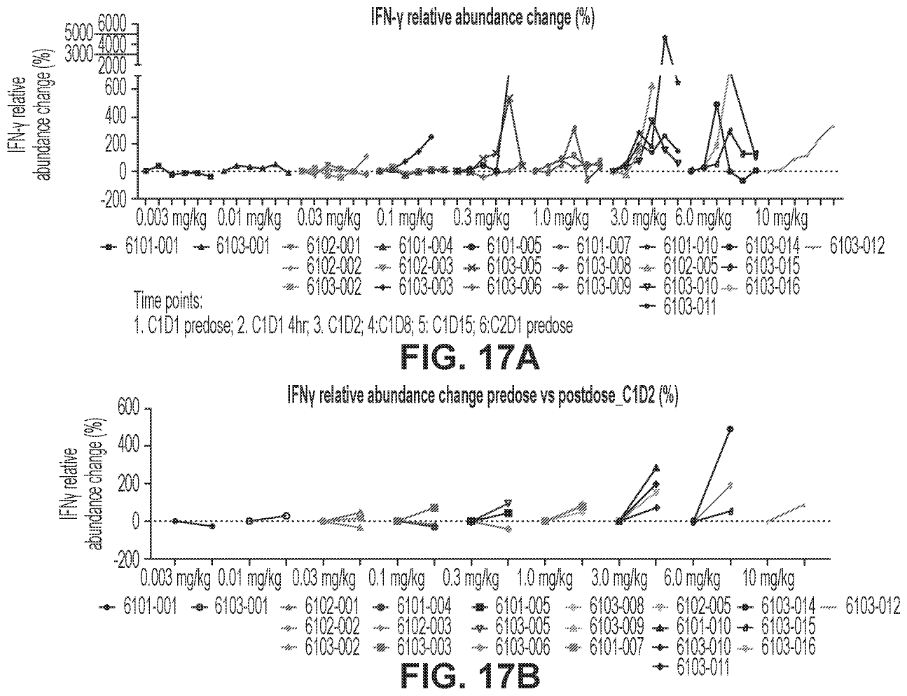

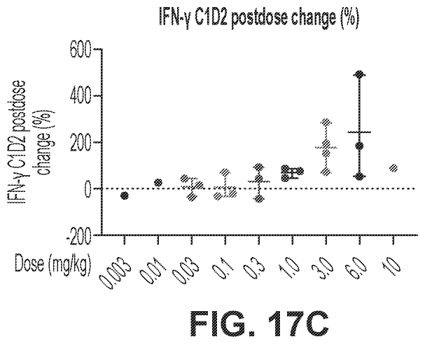

Pharmacodynamic results are presented in FIGS. 17-26. Blood samples of patients at serial visit points were collected for pharmacodynamic study in order to monitor the dynamic changes of cytokines and immune-related soluble proteins. Blood was collected at six visit time points, Cycle 1 Day 1 pre-dose, Cycle 1 Day 1 4 hr, Cycle 1 Day 2, Cycle 1 Day 8, Cycle 1 Day 15, and Cycle 2 Day 1. Serum was prepared following standard protocols. An MSD panel was designed to investigate whether there is a dose-dependent change in the abundance of five cytokines, IFN-γ, IL-2, IL-6, IL-10, and TNF-α, in the peripheral blood during TY21580 treatment. In addition, serum levels of four soluble proteins, sCTLA-4, sPD-L1, sCD25, and CXCL11, were measured.

FIG. 17A shows a dose dependent change in serum IFN-γ concentration in patients treated with TY21580, with IFN-γ levels increasing significantly in the first two days after dosing for most patients. These kinetics reflect immune system activation and may correlate with the antibody concentration in patients' blood. The change in absolute concentration of each analyte was compared to baseline, which shows a clear dose-dependent response of IFN-γ to TY21580 treatment at doses up to 6 mg/kg (FIG. 17B). In addition, quantifying changes in IFN-γ serum concentration at C1D2 from baseline level (C1D1 pre-dose) within each dose group showed the increase in IFN-γ abundance to be dose dependent at 0.1 mg/kg and higher (FIG. 17C), which indicates the immune system is activated upon TY21580 treatment. These data are consistent with one proposed mechanism of TY21580 action, in which TY21580 activates the immune system through relief of CTLA-4-mediated immunosuppression.

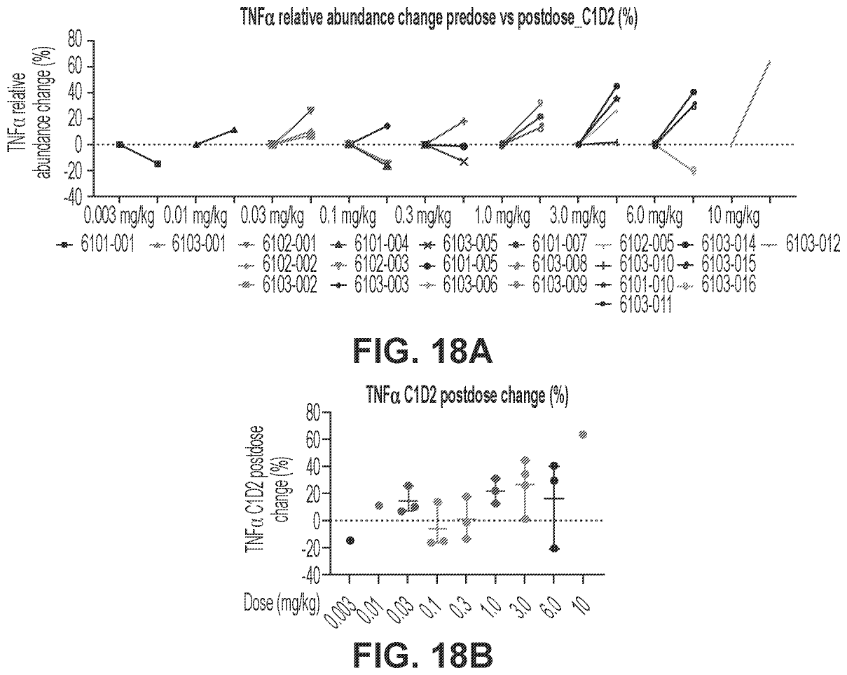

TNFα concentrations were also measured in patients treated with TY2180 and changes were plotted relative to baseline levels. FIG. 18 show a dose dose-dependent increase in TNFα levels following treatment with TY2180.

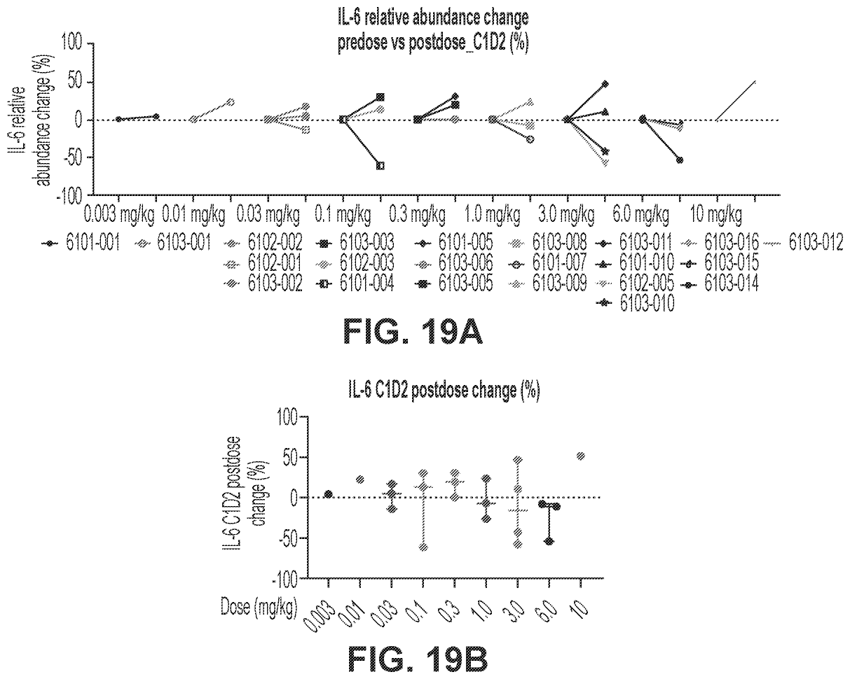

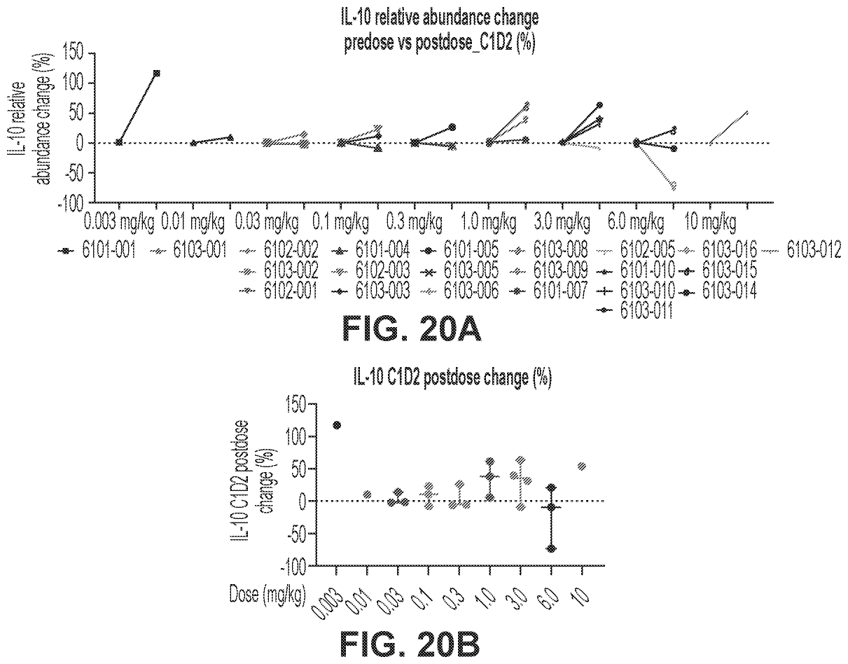

In addition, IL-6 (FIG. 19), IL-10 (FIG. 20), and IL-2 (data not shown) were measured in patients treated with TY2180. FIG. 19 show no observable dose-dependent change in IL-6 concentration in patients treated with TY21580. Serum concentration of IL-2 was below the assay detection limit in most patients.

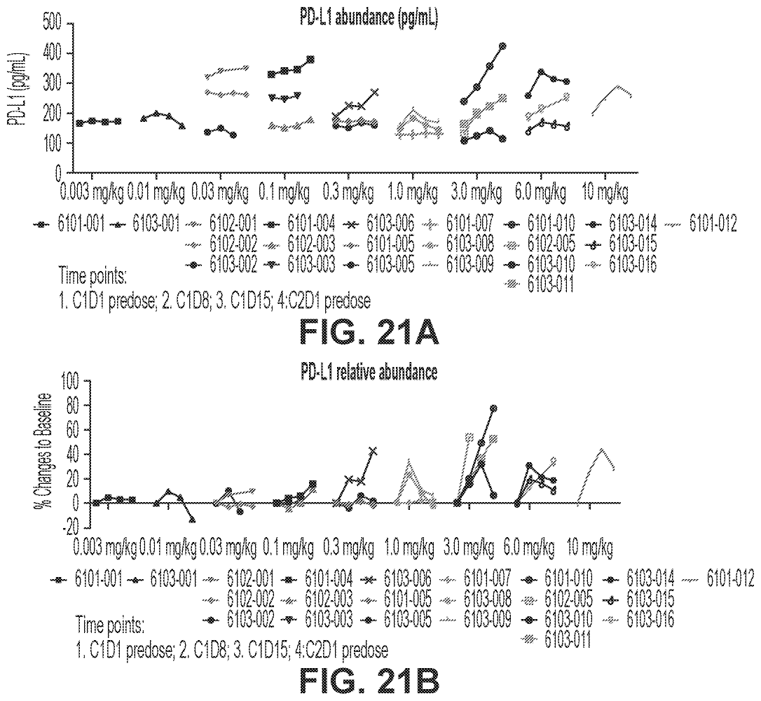

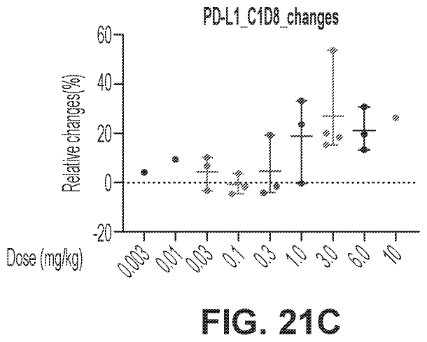

The serum concentration of soluble PD-L1 was also measured in patients treated with TY21580 at serial visit time points (FIG. 21A) and FIG. 21B shows changes in sPD-L1 abundance relative to baseline in response to treatment. Changes of sPD-L1 abundance in each dose group is shown in FIG. 21C. sPD-L1 levels increased significantly in patients dosed with 3 mg/kg and higher, which might indicate a clinical response.

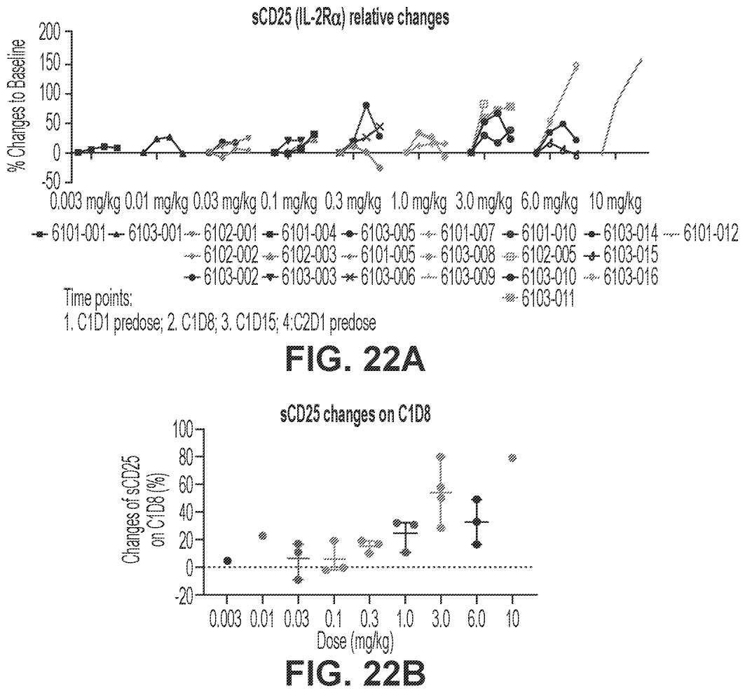

FIG. 22 shows a dose dependent increase in soluble CD25 at C1D8 as compared to baseline levels.

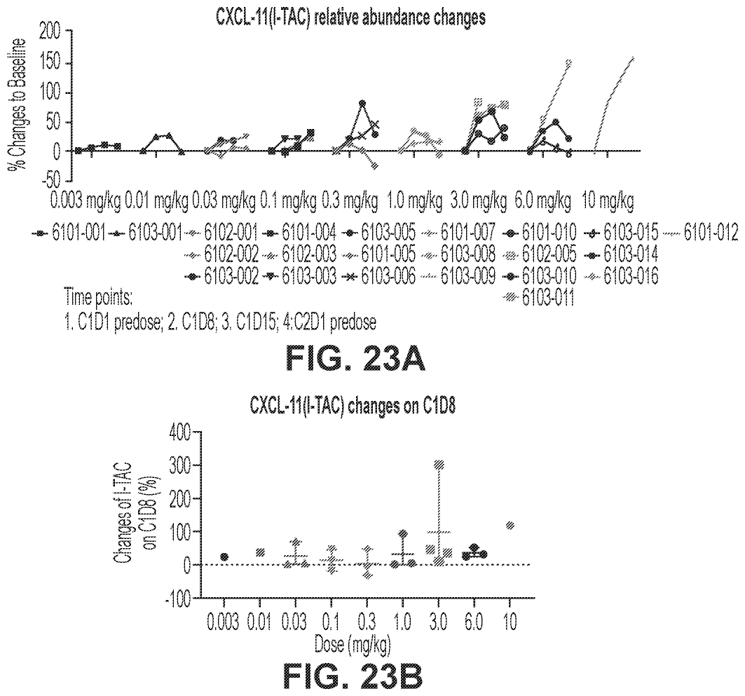

The serum concentration of soluble CXCL11 was also measured in patients treated with TY21580 at serial visit time points, and FIG. 23 shows what appears to be a dose-dependent increase in response following TY21580 treatment. These data show that CXCL11 abundance increases significantly in patients who received a dose equal to or exceeding 0.3 mg/kg.

Whole blood collected from patients was also used to assess T cell and NK cell counts in patients treated with TY21580.

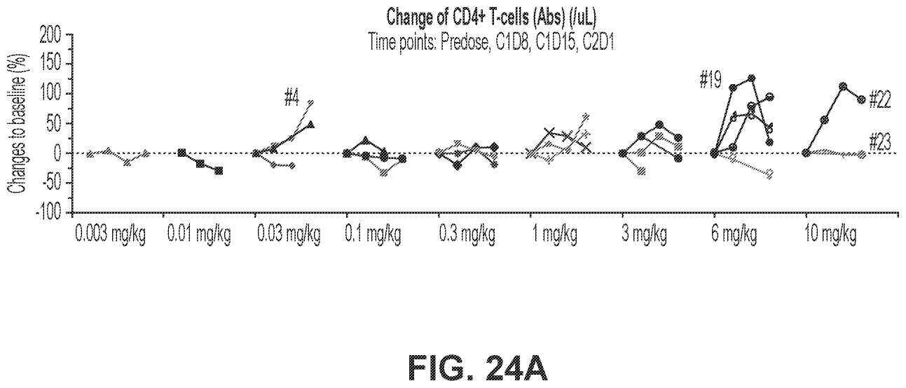

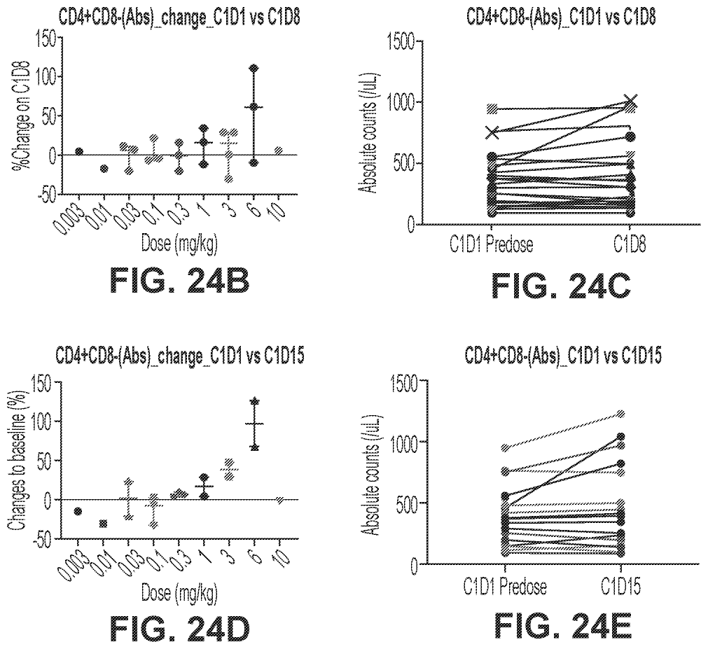

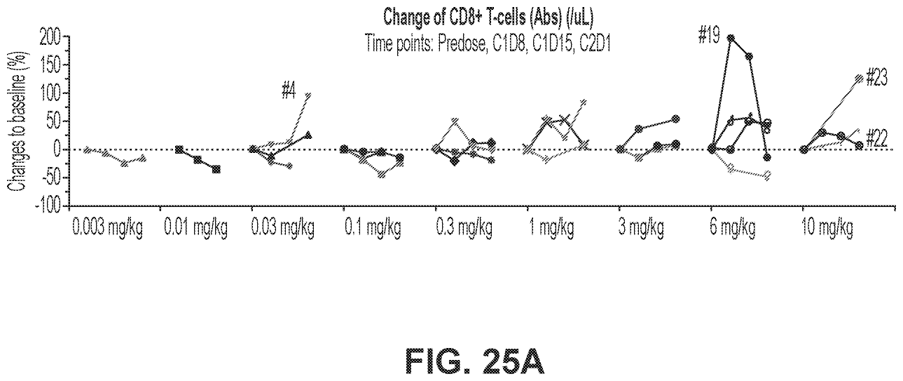

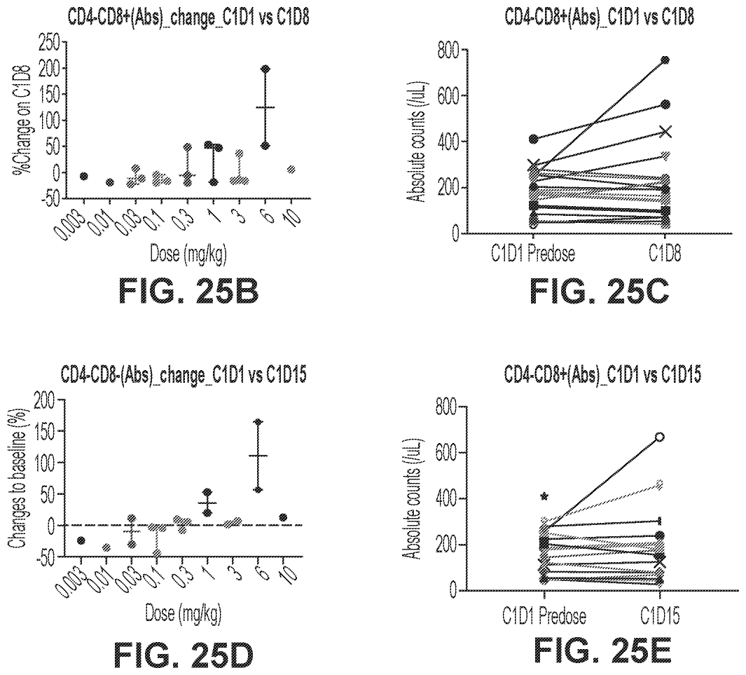

CD4+CD8− T cells were quantified in whole blood collected from patients treated with TY21580 at C1D1 (pre-dose), C1D8, C1D15, and C2D1 time points. A dose-dependent increase of CD4+ cells was measured by absolute count, which was particularly notable at the 3 mg/kg and 6 mg/kg doses (FIG. 24A). FIGS. 24B-24E compare changes in CD4+ T cell counts between baseline and C1D8 (FIGS. 24B and 24C) and baseline and C1D8 (FIGS. 24D and 24E), with FIGS. 24B and 24D showing the relative change compared to baseline, and FIGS. 24C and 24E showing the change in absolute counts per μL. A dose-dependent increase in absolute count of CD8+ T cells was also measured, as seen in FIG. 25. These dose-dependent increases in absolute count of CD4+ and CD8+ T cells indicates the immune system is activated upon TY21580 treatment. This data also shows that even two weeks following treatment (C1D15), TY21580 showed consistent anti-tumor efficacy.

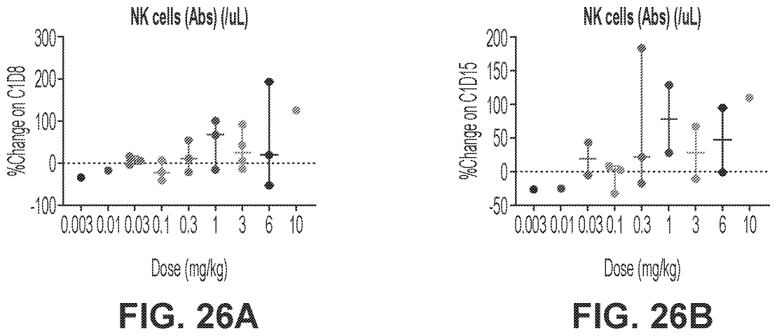

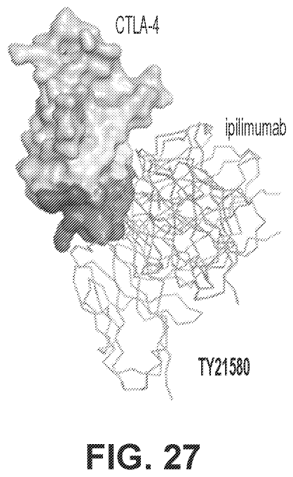

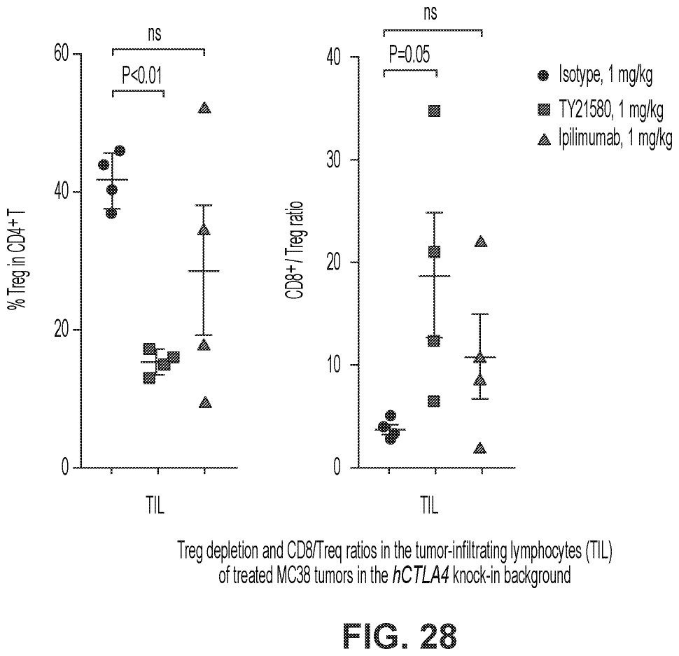

NK cell counts were also determined in patients treated with TY21580, with FIG. 26 showing the change in absolute cell counts per μL at C1D8 and C1D15.

### Interim Efficacy Data

Subject #23 is a 74 year-old male with renal cell carcinoma who relapsed on nivolumab and is enrolled in the 10 mg/kg cohort. CD8+ T cells increased in this subject after the 1st cycle of treatment (FIGS. 15C and 25A), showing that TY21580 is highly active for triggering T cell activation.

Subject #22 is a 77 year-old male with pancreatic cancer in the 10 mg/kg cohort who has undergone three previous therapies. This subject has two target lesions, one in the pancreas and one in the liver with baseline measurements of 35 mm for the pancreas lesion and 15 mm for the liver lesion. The first tumor assessment was performed on this patient after two cycles of TY21580 treatment. This assessment shows the pancreas lesion has shrunk to 29 mm and the liver lesion has shrunk to 10 mm, reflecting a 22% reduction in target lesion size (Table 4).

TABLE 4
Tumor shrinkage in Subject #22 (pancreatic cancer)

| | | | 1st tumor |
|---|---|---|---|
| Patient #22 | | Baseline | assessment |
| Target lesions | TL1-Pancreas | 35 mm | 29 mm |
| | TL-2 Liver | 15 mm | 10 mm |
| Non-target lesion | Portal vein lymph node | Present | Disappear |

| | |
|---|---|
| Change in target | −22% |
| lesions | |

Prolonged stable disease is also observed in 17% (4/24) of patients, all of whom had received multiple prior treatments and had “cold tumors” (i.e., tumors that T cells cannot penetrate because the tumor has not been recognized by the immune system, or provoked an immune response). Notably, all four patients have increased CD8 and/or CD4 T cells, with Subjects #4 and #19 having significantly increased CD8 T cells. (FIGS. 24A and 25A).

### Example 2. Dose Selection Criteria for TY21580

The serum pharmacokinetics (PK) of TY21580 monotherapy after IV infusion was assessed using Non-compartmental Analysis (NCA by Phoenix WinNonlin version 8.3) and population PK modeling (by NONMEM® 7.5) approaches.

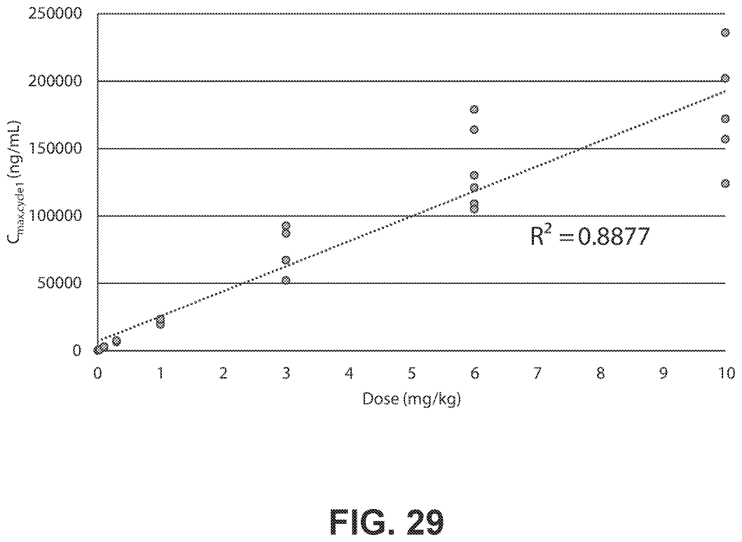

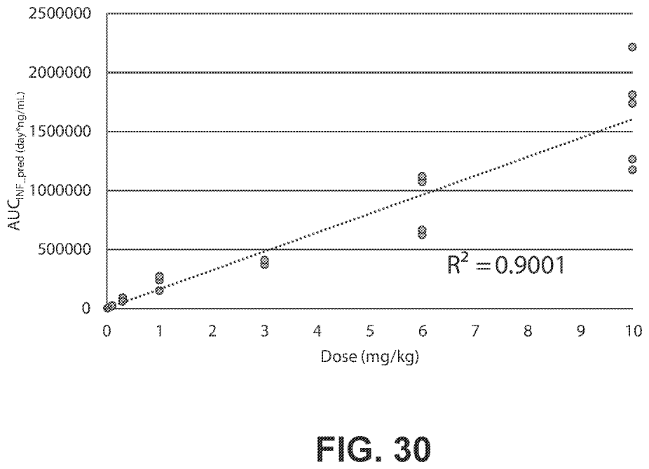

Dose-dependent increase in serum exposure was observed. The maximum serum concentration post IV infusion (C_{max,cycle 1}) has an approximately linear relationship with dose (e.g., 0.003 to 10 mg/kg, R²=0.89, FIG. 29). Predicted drug exposure AUC_{0-infinity} based on cycle 1 PK (AUC_{INF_pred}) has an approximately linear relationship with dose (e.g., 0.01 to 10 mg/kg, R²=0.90, FIG. 30).

Cycle 1 terminal half-life (mean±standard deviation) of TY21580 in serum is estimated as ˜10±3 days from 0.01 to 10 mg/kg. No apparent dose-dependency was observed. This set of PK analysis, including Cₘₐₓ, AUC and half-life indicated that for TY21580, no significant target-mediated drug disposition (TMDD) was observed from 0.01 to 10 mg/kg. A 2-compartment linear population PK model was developed and can describe the PK data reasonably well. Body-weight (BW) is highly correlated with the model-estimated central of volume of distribution (Vc) and is considered as a meaningful covariate (e.g., exponent value >0.8). For patients with PK data from multiple treatment cycles (Q3W, once every three-week dosing), limited accumulation (e.g., <2-fold) for pre-dose PK (C_{trough,repeat dose}) and C_{max,repeat dose} was observed in most patients, consistent with PK model-estimated half-life and predicted mean accumulation ratios (e.g., ˜1.3-fold) with Q3W dosing.

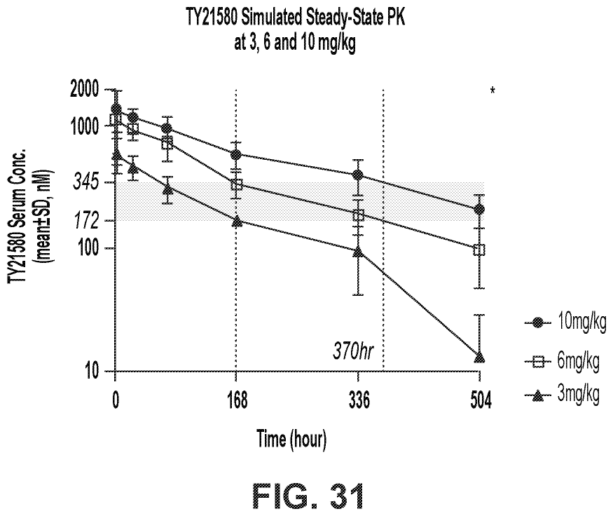

Potentially efficacious human doses based on observed PK vs. in vitro data were explored. EC₉₀ₛ of TY21580 in various binding assays generally are >3-5 μg/mL, consistent with functional readouts and could be targeted as minimal tumor tissue steady-state pre-dose concentrations (i.e. C_{trough,ss}) These EC₉₀ₛ correlate with a theoretical receptor occupancy (RO) of ˜90%. As an example, ˜5 μg/mL (˜34.5 nM) was chosen as a representative integrated EC₉₀ value based on key available in vitro data (e.g., binding, functional readout for T cell activation and ADCC.) This EC₉₀ corresponds to 172-345 nM in the systemic circulation considering a likely 10-20% tumor tissue drug distribution for a target without significant TMDD. At a 10 mg/kg dose of TY21580, the mean observed serum trough concentration of TY21580 on day 21 in the first cycle is still around the lowest targeted systemic concentration (e.g., 172 nM, assuming 20% tumor drug distribution). These PK data, together with simulated PK at steady state (FIG. 31, showing that the C_{trough} conc. at 504 hour of the dosing cycle at 10 mg/kg, e.g., 212 nM, is close to the middle range of the target conc.), monotherapy clinical PD biomarker data, and safety/efficacy data support 10 mg/kg once every three weeks (Q3W) IV dosing as a likely efficacious/saturating monotherapy dose.

Considering the predicted mean accumulation ratios of TY21580 (e.g., ˜1.3-fold) with Q3W dosing, at 6 or 3 mg/kg, mean predicted day 14 or day 7 serum concentrations of TY21580 would be around or slightly above the lowest targeted systemic concentration (e.g., 172 nM), respectively (FIG. 31). Therefore, at steady state with 3 mg/kg Q3W dosing, the estimated time (e.g., −168 hours, FIG. 31) above the lowest target conc. is ˜33% of the total cycle duration, and may be sufficient to allow for optimal therapeutic index. Alternatively, at steady state with 6 mg/kg Q3W dosing, the estimated time (e.g., −370 hours, FIG. 31) above the lowest target concentration is >70% of the total cycle duration.

What is claimed is:

1. A method of treating a cancer in a subject, comprising: (a) administering to the subject an effective amount of an anti-CTLA4 antibody, wherein the antibody specifically binds to an epitope comprising amino acid residues Y105 and L106 of human CTLA4 but does not comprise residue I108, wherein the numbering of the amino acid residues is according to SEQ ID NO: 108, (b) subsequently determining a level of one or more biomarkers selected from the group consisting of IL-1β, IL-2, IL-6, IL-10, interferon (IFN)-γ, tumor necrosis factor (TNF)-α, soluble CTLA4 (sCTLA4), soluble PD-L1 (sPD-L1), soluble CD25 (sCD25), CXCL11, FoxP3, Ki67, CD8+ T cells, CD4+ T cells, CD8+ effector memory T (Tₑₘ) cells, CD4+ Tₑₘ cells, regulatory T (T_{reg}) cells, a ratio of CD8+ Tₑₘ cells to T_{reg} cells, a ratio of CD4+ Tₑₘ cells to T_{reg} cells, NK cells, B cells in a sample of the subject.

2. The method of claim 1, wherein an increase of the level of one or more biomarkers selected from the group consisting of CD8+ T cells, CD4+ T cells, CD8+ Tₑₘ cells, CD4+ Tₑₘ cells, a ratio of CD8+ Tₑₘ cells to T_{reg} cells, a ratio of CD4+ Tₑₘ cells to T_{reg} cells, NK cells and B cells after administration of the anti-CTLA4 antibody compared to the baseline level of the one or more biomarkers indicates an increased likelihood that the subject has an effective response to the CTLA4 antibody.

3. The method of claim 1 or 2, wherein the sample has an increase of the level of one or more biomarkers selected from the group consisting of CD8+ T cells, CD4+ T cells, CD8+ Tₑₘ cells, CD4+ Tₑₘ cells, T_{reg} cells, a ratio of CD8+ Tₑₘ cells to T_{reg} cells, a ratio of CD4+ Tₑₘ cells to T_{reg} cells, NK cells and B cells after administration of the anti-CTLA4 antibody compared to the baseline level of the one or more biomarkers, the method further comprises administering to the subject a further cycle of an effective amount of the anti-CTLA4 antibody.

4. The method of any one of claims 1-3, wherein a decrease of the level of T_{reg} cells after administration of the anti-CTLA4 antibody compared to the baseline level of the T_{reg} cells indicates an increased likelihood that the subject has an effective response to the CTLA4 antibody.

5. The method of any one of claims 1-4, wherein the sample has a decrease of the level of T_{reg} cells after administration of the anti-CTLA4 antibody compared to the baseline level of the T_{reg} cells, the method further comprises administering to the subject a further cycle of an effective amount of the anti-CTLA4 antibody.

6. A method of providing a prognosis for a subject who has been administered with an effective amount of an anti-CTLA4 antibody, wherein the antibody specifically binds to an epitope comprising amino acid residues Y105 and L106 of human CTLA4 but does not comprise residue I108, wherein the numbering of the amino acid residues is according to SEQ ID NO: 108; the method comprising determining a level of one or more biomarkers selected from the group consisting of CD8+ T cells, CD4+ T cells, CD8+ Tₑₘ cells, CD4+ Tₑₘ cells, T_{reg} cells, a ratio of CD8+ Tₑₘ cells to T_{reg} cells, a ratio of CD4+ Tₑₘ cells to T_{reg} cells, NK cells, B cells in a sample of the subject, wherein: (a) an increase of the level of one or more biomarkers selected from the group consisting of CD8+ T cells, CD4+ T cells, CD8+ Tₑₘ cells, CD4+ Tₑₘ cells, a ratio of CD8+ Tₑₘ cells to T_{reg} cells, a ratio of CD4+ Tₑₘ cells to T_{reg} cells, NK cells and B cells after administration of the anti-CTLA4 antibody compared to the baseline level of the one or more biomarkers indicates an increased likelihood that the subject has an effective response to the CTLA4 antibody; and/or (b) a decrease of the level of T_{reg} cells after administration of the anti-CTLA4 antibody compared to the baseline level of the T_{reg} cells indicates an increased likelihood that the subject has an effective response to the CTLA4 antibody.

7. The method of any one of claims 1-6, wherein the one or more biomarkers comprise CD8+ Tₑₘ cells.

8. The method of any one of claims 1-7, wherein the one or more biomarkers comprise CD4+ Tₑₘ cells.

9. The method of any one of claims 1-8, wherein the one or more biomarkers comprise NK cells.

10. The method of any one of claims 1-9, wherein the one or more biomarkers comprise T_{reg} cells.

11. The method of any one of claims 1-10, wherein the one or more biomarkers comprise a ratio of CD8+ Tₑₘ cells to T_{reg} cells.

12. The method of any one of claims 1-11, wherein the one or more biomarkers comprise a ratio of CD4+ Tₑₘ cells to T_{reg} cells.

13. The method of any one of claims 1-12, wherein the sample is a blood sample.

14. The method of any one of claims 1-12, wherein the sample is a tumor biopsy sample.

15. The method of any one of claims 1-14, wherein the cancer is resistant or refractory to a prior therapy, wherein the prior therapy is an inhibitor of CTLA4, PD-1, or a PD-1 ligand.

16. A method of treating a cancer in a subject, comprising administering to the subject an effective amount of an anti-CTLA4 antibody, wherein the antibody specifically binds to an epitope comprising amino acid residues Y105 and L106 of human CTLA4 but does not comprise residue I108, wherein the numbering of the amino acid residues is according to SEQ ID NO: 108, and wherein the cancer is resistant or refractory to a prior therapy, wherein the prior therapy is an inhibitor of CTLA4, PD-1, or a PD-1 ligand.

17. The method of claim 15 or 16, wherein the prior therapy is an anti-PD-1 antibody.

18. The method of claim 17, wherein the anti-PD-1 antibody is pembrolizumab or nivolumab.

19. The method of any one of claims 1-18, wherein the cancer is a solid cancer.

20. The method of claim 19, wherein the cancer is urothelial carcinoma.

21. The method of claim 19, wherein the cancer is renal cell carcinoma.

22. The method of claim 19, wherein the cancer is pancreatic cancer

23. The method of any one of claims 1-22, wherein the cancer is advanced-stage cancer.

24. The method of any one of claims 1-23, wherein the cancer is metastatic cancer.

25. The method of any one of claims 1-24, wherein the anti-CTLA4 antibody is administered at a dose of about 0.001 mg/kg to about 15 mg/kg.

26. The method of any one of claims 1-24, wherein the anti-CTLA4 antibody is administered at a dose of about 0.001 mg/kg to about 10 mg/kg.

27. The method of any one of claims 1-24, wherein the anti-CTLA4 antibody is administered at a dose of at least about 0.03 mg/kg, about 1 mg/kg, about 3 mg/kg. about 6 mg/kg, about 10 mg/kg, or about 15 mg/kg.

28. The method of any one of claims 1-24, wherein the anti-CTLA4 antibody is administered at a dose of at least about 3 mg/kg or at least about 10 mg/kg.

29. The method of any one of claims 1-24, wherein the anti-CTLA4 antibody is administered at a dose of at about 3 mg/kg.

30. The method of any one of claims 1-24, wherein the anti-CTLA4 antibody is administered at a dose of at about 6 mg/kg.

31. The method of any one of claims 1-24, wherein the anti-CTLA4 antibody is administered at a dose of at about 10 mg/kg.

32. The method of any one of claims 1-31, wherein the anti-CTLA4 antibody is administered intravenously.

33. The method of any one of claims 1-32, wherein the anti-CTLA4 antibody is administered about once every three weeks.

34. The method of any one of claims 1-33, wherein the subject receives at least 4 cycles of treatment with the anti-CTLA4 antibody.

35. The method of claim 34, wherein the subject further receives a maintenance treatment comprising administering to the subject an effective amount of the anti-CTLA4 antibody about once every four weeks to about once every twelve weeks.

36. The method of any one of claims 1-35, wherein the subject is human.

37. The method of any one of claims 1-36, wherein;
a) the antibody binds to human CTLA4, cynomolgus monkey CTLA4, mouse CTLA4, rat CTLA4, and dog CTLA4 with a dissociation constant (K_{D}) of about 350 nM or less;
b) binding of the anti-CTLA4 antibody induces antibody-dependent cell cytotoxicity (ADCC) against a CTLA4-expressing human cell or a human T_{reg} cell, wherein the ADCC activity of the anti-CTL4 antibody is higher than the ADCC activity of ipilimumab; and/or
c) the anti-CTLA4 antibody has an IC50 higher than the IC50 of ipilimumab for blocking binding of CD80 and/or CD86 to human CTLA4 in an assay wherein either when CD80 and/or CD86 are plate bound or when human CTLA4 is present on cell surface.

38. The method of any one of claims 1-37, wherein the antibody comprises a heavy chain variable region and a light chain variable region,
a) wherein the heavy chain variable region comprises an HVR-H1, an HVR-H2, and an HVR-H3,
wherein the HVR-H1 comprises an amino acid sequence according to a formula selected from the group consisting of:
Formula (I): X1TFSX2YX3IHWV (SEQ ID NO: 1), wherein X1 is F or Y, X2 is D or G, and X3 is A, G, or W;
Formula (II): YSIX1SGX2X3WX4WI (SEQ ID NO: 2), wherein X1 is S or T, X2 is H or Y, X3 is H or Y, and X4 is A, D, or S; and
Formula (III): FSLSTGGVAVX1W I (SEQ ID NO: 3), wherein X1 is G or S;
wherein the HVR-H2 comprises an amino acid sequence according to a formula selected from the group consisting of:
Formula (IV): IGX1IX2HSGSTYYSX3SLKSRV (SEQ ID NO: 4), wherein X1 is D or E, X2 is S or Y, and X3 is P or Q;
Formula (V): IGX1ISPSX2GX3TX4YAQKFQGRV (SEQ ID NO: 5), wherein X1 is I or W, X2 is G or S, X3 is G or S, and X4 is K or N; and
Formula (VI): VSX1ISGX2GX3X4TYYADSVKGRF (SEQ ID NO: 6), wherein X1 is A, G, or S, X2 is S or Y, X3 is G or S, and X4 is S or T; and
wherein the HVR-H3 comprises an amino acid sequence according to a formula selected from the group consisting of:
Formula (VII): ARX1X2X3X4FDX5 (SEQ ID NO: 7), wherein X1 is G, R, or S, X2 is A, I, or Y, X3 is D, V, or Y, X4 is A, E, or Y, and X5 is I or Y;
Formula (VIII): ARX1GX2GYFDX3 (SEQ ID NO: 8), wherein X1 is D or L, X2 is F or Y, and X3 is V or Y;
Formula (IX): ARX1X2X3X4AX5X6FDY (SEQ ID NO: 9), wherein X1 is L or R, X2 is I or P, X3 is A or Y, X4 is S or T, X5 is T or Y, and X6 is A or Y;
Formula (X): ARDX1X2X3GSSGYYX4GFDX5 (SEQ ID NO: 10), wherein X1 is I or V, X2 is A or H, X3 is P or S, X4 is D or Y, and X5 is F or V; and
b) wherein the light chain variable region comprises an HVR-L1, an HVR-L2, and an HVR-L3,
wherein the HVR-L1 comprises an amino acid sequence according to a formula selected form the group consisting of:
Formula (XI): RASQX1X2X3SX4LX5 (SEQ ID NO: 11), wherein X1 is G or S, X2 is I or V, X3 is G or S, X4 is S or Y, and X5 is A or N;
Formula (XII): RASQX1VX2X3RX4LA (SEQ ID NO: 12), wherein X1 is S or T, X2 is F, R, or S, X3 is G or S, and X4 is F or Y; and
Formula (XIII): RASX1SVDFX2GX3SFLX4 (SEQ ID NO: 13), wherein X1 is E or Q, X2 is D, F, H, or Y, X3 is F, I, or K, and X4 is A, D, or H;
wherein the HVR-L2 comprises an amino acid sequence according to Formula (XIV): X1ASX2X3X4X5GX6 (SEQ ID NO: 14), wherein X1 is A or D, X2 is N, S, or T, X3 is L or R, X4 is A, E, or Q, X5 is S or T, and X6 is I or V; and
wherein the HVR-L3 comprises an amino acid sequence according to a formula selected from the group consisting of:
Formula (XV): YCX1X2X3X4X5X6PX7T (SEQ ID NO: 15), wherein X1 is E, Q, or V, X2 is H or Q, X3 is A, G, H, R, or S, X4 is D, L, S, or Y, X5 is E, G, P, Q, or S, X6 is L, T, V, or W, and X7 is F, L, P, W, or Y;
Formula (XVI): YCQQX1X2X3WPPWT (SEQ ID NO: 16), wherein X1 is S or Y, X2 is D or Y, and X3 is Q or Y; and
Formula (XVII): YCQX1YX2SSPPX3YT (SEQ ID NO: 17), wherein X1 is H or Q, X2 is T or V, and X3 is E or V.

39. The method of claim 38, wherein the antibody comprises:
a) an HVR-H1 comprising the amino acid sequence of SEQ ID NO: 18, an HVR-H2 comprising the amino acid sequence of SEQ ID NO: 30, an HVR-H3 comprising the amino acid sequence of SEQ ID NO: 40, an HVR-L1 comprising the amino acid sequence of SEQ ID NO: 53, an HVR-L2 comprising the amino acid sequence of SEQ ID NO: 66, and an HVR-L3 comprising the amino acid sequence of SEQ ID NO: 70;
b) an HVR-H1 comprising the amino acid sequence of SEQ ID NO: 19, an HVR-H2 comprising the amino acid sequence of SEQ ID NO: 31, an HVR-H3 comprising the amino acid sequence of SEQ ID NO: 41, an HVR-L1 comprising the amino acid sequence of SEQ ID NO: 54, an HVR-L2 comprising the amino acid sequence of SEQ ID NO: 67, and an HVR-L3 comprising the amino acid sequence of SEQ ID NO: 71;
c) an HVR-H1 comprising the amino acid sequence of SEQ ID NO: 20, an HVR-H2 comprising the amino acid sequence of SEQ ID NO: 32, an HVR-H3 comprising the amino acid sequence of SEQ ID NO: 42, an HVR-L1 comprising the amino acid sequence of SEQ ID NO: 55, an HVR-L2 comprising the amino acid sequence of SEQ ID NO: 66, and an HVR-L3 comprising the amino acid sequence of SEQ ID NO: 72;
d) an HVR-H1 comprising the amino acid sequence of SEQ ID NO: 21 an HVR-H2 comprising the amino acid sequence of SEQ ID NO: 33, an HVR-H3 comprising the amino acid sequence of SEQ ID NO: 43, an HVR-L1 comprising the amino acid sequence of SEQ ID NO: 56, an HVR-L2 comprising the amino acid sequence of SEQ ID NO: 68, and an HVR-L3 comprising the amino acid sequence of SEQ ID NO: 73;
e) an HVR-H1 comprising the amino acid sequence of SEQ ID NO: 22, an HVR-H2 comprising the amino acid sequence of SEQ ID NO: 34, an HVR-H3 comprising the amino acid sequence of SEQ ID NO: 44, an HVR-L1 comprising the amino acid sequence of SEQ ID NO: 57, an HVR-L2 comprising the amino acid sequence of SEQ ID NO: 66, and an HVR-L3 comprising the amino acid sequence of SEQ ID NO: 74;
f) an HVR-H1 comprising the amino acid sequence of SEQ ID NO: 23, an HVR-H2 comprising the amino acid sequence of SEQ ID NO: 35, an HVR-H3 comprising the amino acid sequence of SEQ ID NO: 45, an HVR-L1 comprising the amino acid sequence of SEQ ID NO: 58, an HVR-L2 comprising the amino acid sequence of SEQ ID NO: 66, and an HVR-L3 comprising the amino acid sequence of SEQ ID NO: 75,
g) an HVR-H1 comprising the amino acid sequence of SEQ ID NO: 24, an HVR-H2 comprising the amino acid sequence of SEQ ID NO: 32, an HVR-H3 comprising the amino acid sequence of SEQ ID NO: 46, an HVR-L1 comprising the amino acid sequence of SEQ ID NO: 59, an HVR-L2 comprising the amino acid sequence of SEQ ID NO: 66, and an HVR-L3 comprising the amino acid sequence of SEQ ID NO: 76;
h) an HVR-H1 comprising the amino acid sequence of SEQ ID NO: 25, an HVR-H2 comprising the amino acid sequence of SEQ ID NO: 36, an HVR-H3 comprising the amino acid sequence of SEQ ID NO: 47, an HVR-L1 comprising the amino acid sequence of SEQ ID NO: 60, an HVR-L2 comprising the amino acid sequence of SEQ ID NO: 69, and an HVR-L3 comprising the amino acid sequence of SEQ ID NO: 77;
i) an HVR-H1 comprising the amino acid sequence of SEQ ID NO: 26, an HVR-H2 comprising the amino acid sequence of SEQ ID NO: 37, an HVR-H3 comprising the amino acid sequence of SEQ ID NO: 48, an HVR-L1 comprising the amino acid sequence of SEQ ID NO: 61, an HVR-L2 comprising the amino acid sequence of SEQ ID NO: 66, and an HVR-L3 comprising the amino acid sequence of SEQ ID NO: 78;
j) an HVR-H1 comprising the amino acid sequence of SEQ ID NO: 27, an HVR-H2 comprising the amino acid sequence of SEQ ID NO: 32, an HVR-H3 comprising the amino acid sequence of SEQ ID NO: 49, an HVR-L1 comprising the amino acid sequence of SEQ ID NO: 62, an HVR-L2 comprising the amino acid sequence of SEQ ID NO: 67, and an HVR-L3 comprising the amino acid sequence of SEQ ID NO: 79;
k) an HVR-H1 comprising the amino acid sequence of SEQ ID NO: 28, an HVR-H2 comprising the amino acid sequence of SEQ ID NO: 37, an HVR-H3 comprising the amino acid sequence of SEQ ID NO: 50, an HVR-L1 comprising the amino acid sequence of SEQ ID NO: 63, an HVR-L2 comprising the amino acid sequence of SEQ ID NO: 67, and an HVR-L3 comprising the amino acid sequence of SEQ ID NO: 80;
l) an HVR-H1 comprising the amino acid sequence of SEQ ID NO: 18, an HVR-H2 comprising the amino acid sequence of SEQ ID NO: 38, an HVR-H3 comprising the amino acid sequence of SEQ ID NO: 51, an HVR-L1 comprising the amino acid sequence of SEQ ID NO: 64, an HVR-L2 comprising the amino acid sequence of SEQ ID NO: 67, and an HVR-L3 comprising the amino acid sequence of SEQ ID NO: 81; or
m) an HVR-H1 comprising the amino acid sequence of SEQ ID NO: 29, an HVR-H2 comprising the amino acid sequence of SEQ ID NO: 39, an HVR-H3 comprising the amino acid sequence of SEQ ID NO: 52, an HVR-L1 comprising the amino acid sequence of SEQ ID NO: 65, an HVR-L2 comprising the amino acid sequence of SEQ ID NO: 68, and an HVR-L3 comprising the amino acid sequence of SEQ ID NO: 77.

40. The method of claim 38 or 39, wherein the antibody comprises:
a) a heavy chain variable region comprising the amino acid sequence of SEQ ID NO: 82, and a light chain variable region comprising the amino acid sequence of SEQ ID NO: 95;
b) a heavy chain variable region comprising the amino acid sequence of SEQ ID NO: 83, and a light chain variable region comprising the amino acid sequence of SEQ ID NO: 96;
c) a heavy chain variable region comprising the amino acid sequence of SEQ ID NO: 84, and a light chain variable region comprising the amino acid sequence of SEQ ID NO: 97;
d) a heavy chain variable region comprising the amino acid sequence of SEQ ID NO: 85, and a light chain variable region comprising the amino acid sequence of SEQ ID NO: 98;
e) a heavy chain variable region comprising the amino acid sequence of SEQ ID NO: 86, and a light chain variable region comprising the amino acid sequence of SEQ ID NO: 99;
f) a heavy chain variable region comprising the amino acid sequence of SEQ ID NO: 87, and a light chain variable region comprising the amino acid sequence of SEQ ID NO: 100;
g) a heavy chain variable region comprising the amino acid sequence of SEQ ID NO: 88, and a light chain variable region comprising the amino acid sequence of SEQ ID NO: 101;
h) a heavy chain variable region comprising the amino acid sequence of SEQ ID NO: 89, and a light chain variable region comprising the amino acid sequence of SEQ ID NO: 102;
i) a heavy chain variable region comprising the amino acid sequence of SEQ ID NO: 90, and a light chain variable region comprising the amino acid sequence of SEQ ID NO: 103;
j) a heavy chain variable region comprising the amino acid sequence of SEQ ID NO: 91, and a light chain variable region comprising the amino acid sequence of SEQ ID NO: 104;
k) a heavy chain variable region comprising the amino acid sequence of SEQ ID NO: 92, and a light chain variable region comprising the amino acid sequence of SEQ ID NO: 105;
l) a heavy chain variable region comprising the amino acid sequence of SEQ ID NO: 93, and a light chain variable region comprising the amino acid sequence of SEQ ID NO: 106; or
m) a heavy chain variable region comprising the amino acid sequence of SEQ ID NO. 94, and a light chain variable region comprising the amino acid sequence of SEQ ID NO: 107.

41. The method of any one of claims 1-37, wherein the antibody comprises: (a) a heavy chain variable region comprising an HVR-H1 comprising the amino acid sequence of SEQ ID NO: 23, an HVR-H2 comprising the amino acid sequence of SEQ ID NO: 35, and an HVR-H3 comprising the amino acid sequence of SEQ ID NO: 45, and/or a light chain variable region comprising an HVR-L1 comprising the amino acid sequence of SEQ ID NO: 58, an HVR-L2 comprising the amino acid sequence of SEQ ID NO: 66, and an HVR-L3 comprising the amino acid sequence of SEQ ID NO: 75; or (b) a heavy chain variable region comprising the amino acid sequence of SEQ ID NO: 87 or an amino acid sequence having at least 90% sequence identity to the amino acid sequence of SEQ ID NO: 87, and/or a light chain variable region comprising the amino acid sequence of SEQ ID NO: 100 or an amino acid sequence having at least 90% sequence identity to the amino acid sequence of SEQ ID NO: 100.

42. The method of any one of claims 1-37, wherein the antibody comprises a heavy chain comprising the amino acid sequence of SEQ ID NO: 125 and a light chain comprising the amino acid sequence of SEQ ID NO: 127.

43. The method of any one of claims 1-37, wherein the antibody comprises a heavy chain comprising the amino acid sequence of SEQ ID NO: 126 and a light chain comprising the amino acid sequence of SEQ ID NO: 127.

44. The method of any one of claim 41-43, wherein the antibody is a human antibody.

45. The method of any one of claims 1-44, wherein the antibody comprises a human IgG1 Fc region or a variant that has enhanced ADCC activity.

46. The method of any one of claims 1-45, further comprising administering to the subject a therapeutically effective amount of at least one additional therapeutic agent.
